SEQUENCE LISTING (10 sequences)

SEQ ID NO: 1 - DNA, 22 bp, Artificial Sequence
  Synthetic Polynucleotide
agatgacgtgatcaaaagagca

SEQ ID NO: 2 - DNA, 22 bp, Artificial Sequence
  Synthetic Polynucleotide
cagacatcttgaatccaccaaa

SEQ ID NO: 3 - DNA, 20 bp, Artificial Sequence
  Synthetic Polynucleotide
atggcatacctcaacgccaa

SEQ ID NO: 4 - DNA, 20 bp, Artificial Sequence
  Synthetic Polynucleotide
aaggacttgctcgttggaca

SEQ ID NO: 5 - DNA, 20 bp, Artificial Sequence
  Synthetic Polynucleotide
gggaaagagggtcctgaaac

SEQ ID NO: 6 - DNA, 20 bp, Artificial Sequence
  Synthetic Polynucleotide
agcacgtgggactggtagat

SEQ ID NO: 7 - DNA, 20 bp, Artificial Sequence
  Synthetic Polynucleotide
agtctgcctgctcatcctgt

SEQ ID NO: 8 - DNA, 20 bp, Artificial Sequence
  Synthetic Polynucleotide
ttatctgcatcggcttcgtg

SEQ ID NO: 9 - DNA, 20 bp, Artificial Sequence
  Synthetic Polynucleotide
cacactgtgcccatctacga

SEQ ID NO: 10 - DNA, 20 bp, Artificial Sequence
  Synthetic Polynucleotide
tagctcttctccagggagga

(19) United States
(12) Patent Application Publication
(10) Pub. No.: US 2020/0271660 A1
(43) Pub. Date: Aug. 27, 2020

(54) METHODS AND DEVICES FOR DETECTING BIOMARKERS ASSOCIATED WITH PREECLAMPSIA

(71) Applicant: IGENOMIX S.L., Valencia (ES)

(72) Inventors: Carlos Simon, Valencia (ES); Susan Fisher, San Francisco, CA (US); Tamara Garrido, Valencia (ES)

(73) Assignee: IGENOMIX S.L., Valencia (ES)

(21) Appl. No.: 16/644,544

(22) PCT Filed: Sep. 5, 2018
(86) PCT No.: PCT/IB2018/001117

§ 371 Date: Mar. 5, 2020

Related U.S. Application Data
(60) Provisional application No. 62/554,471, filed on Sep. 5, 2017.

(51) Int. Cl. G01N 33/68; C12Q 1/686; C12Q 1/6813; G01N 33/543

(52) U.S. Cl. CPC G01N 33/689; C12Q 1/686; C12Q 1/6813; G01N 33/54306; G01N 2800/368; G01N 2333/90203; C12Q 2561/113; C12Q 2565/626; G01N 2333/4745; G01N 2333/5756; C12Q 2600/158

(57) ABSTRACT

Provided herein, in some embodiments, are methods and compositions for detecting differentially expressed genes in a sample obtained from a subject having or at risk for preeclampsia.

### RELATED APPLICATIONS

This application claims priority to U.S. Provisional Patent Application No. 62/554,471, filed Sep. 5, 2017, the contents of which are incorporated herein by reference.

### FIELD OF THE INVENTION

Methods and compositions described herein relate to detecting differentially expressed genes (e.g., biomarkers) indicative of having or being at risk for having preeclampsia.

### BACKGROUND OF THE INVENTION

Preeclampsia (PE), which affects ˜8% of first-time pregnancies, impacts 8 million mother-infant pairs worldwide each year (Winn et al., Pregnancy Hypertens, 2011, 1(1):100-108; Fisher, Am J Obstet Gynecol, 2015, 213(4 Suppl):5115-122). This complication, which is specific to human pregnancy, is characterized by the new onset of hypertension, proteinuria and other signs of maternal vascular damage such as edema (Roberts et al., Lancet, 2001, 357(9249):53-56). Severe preeclampsia (sPE) is diagnosed based on a further elevation of blood pressure (systolic ≥160 mm Hg or diastolic of ≥100 mm Hg) or any of the following: thrombocytopenia, impaired liver function, progressive renal insufficiency, pulmonary edema and the new onset of cerebral or visual disturbances (Gynecologists ACoOa & Pregnancy TFoHi, Obstet Gynecol, 2013, 122(5):1122-1131). Currently, a typical cure is delivery of the placenta, and therefore, the infant. As a result, preeclampsia accounts for 15% of preterm births in the U.S. Despite decades of research, a full understanding of PE pathogenesis remains elusive, which contributes to the difficulties involved in the identification of predictive biomarkers and the development of targeted therapeutic strategies.

### SUMMARY OF THE INVENTION

The present disclosure is based, in part, on the finding that certain genes (e.g., biomarkers) are differentially expressed in women that had preeclampsia (PE) in a previous pregnancy compared to women that had a normal pregnancy.

Accordingly, aspects of the disclosure provide methods and compositions for detecting differentially expressed genes (e.g., biomarkers), wherein differentially expressed genes are indicative of having or at risk for having preeclampsia.

In some embodiments, a method for detecting a level of at least one biomarker associated with preeclampsia in a sample from a subject involves (a) determining a level of at least one biomarker in a sample obtained from a subject, wherein the at least one biomarker is selected from the group consisting essentially of: CNR1, IRS2, CHST7, PRUNE2, ADAMTS8, SCARA5, SERPINA3, NPR1, LPAR1, ABLIM2, CHI3L2, LTBP1, TNFRSF8, SLC27A3, IL1, CCDC, PPAP2C, SERTADA4, COCH, FBXO2, Clorf133, and CNIH3; and (b) determining that an absolute value of a ratio of the determined level of the biomarker in the sample to a control level of the biomarker is at least 2, thereby determining that the subject has or is at risk for preeclampsia.

In some embodiments, a method for detecting a level of at least one biomarker associated with preeclampsia in a sample from a subject involves (a) determining a level of at least one biomarker in a sample obtained from a subject, wherein the at least one biomarker is selected from the group consisting essentially of: HSD17B2, ANGPT2, NCKAP5, ADRA2A, DBC1, C1QTNF7, COL8A1, EGR1, SSTR1, FBXO2, CPE, C4orf49, GRP, IGFBP5, COCH, ARHGDIB, SCG5, ITGA11, SLC35F3, RLN2, COL14A1, CLIC2, TMEM25, CCDC81, MYCN, NPR1, RASGRP2, CHI3L2, RSPO3, Cl0orf10, TMEM132C, PPAP2B, NKAIN1, ADAMTS8, IL15, SLC7A2, SERPINA3, NPTX1, CHST7, GALNTL2, SBSN, EDNRA, IL1B, SPARCL1, SCARA5, SIPA1L2, CCL8, P2RY14, CNR1, and IGFBP1; and (b) determining that an absolute value of a ratio of the determined level of the biomarker in the sample to a control level of the biomarker is at least 2, thereby determining that the subject has or is at risk for preeclampsia.

In some embodiments, a method for detecting a level of at least one biomarker associated with preeclampsia in a sample from a subject involves (a) determining a level of at least one biomarker in a sample obtained from a subject, wherein the at least one biomarker is selected from the group consisting essentially of: A1BG-AS1, ARL5B, BAC1-AS, C7, COL8A1, CP, CSPG4, CYP19A1, DEFB1, ENPP4, IPW, LOC101928439, LOC101929607, LOC644172, MIR365A, MIR4509-1, MIR548H1, MME-AS1, MS4A2, OGN, PRKXP1, PSMD3, RNA5SP187, RNA5SP463, RNU2-5P, RNU4-39P, RNU4-76P, RNU4ATAC1BP, RNU6-1111P, RNU6-521P, RNU6-540R, RNU6V, RNUC-901P, RP11-1026M7.3, RP11-106K3.1, RP11-12D16.2, RP11-661Al2.4, RP11-872017.8, SNORD115-32, SNORD52, SNORD71, SPINK1, TAS2R46, TRAJ59, TRBV4-2, TRIM48, TSPAN1, UGT2B7, and ZNF483; (b) determining that an absolute value of a ratio of the determined level of the biomarker in the sample to a control level of the biomarker is at least 2, thereby determining that the subject has or is at risk for preeclampsia.

In some embodiments, a method for detecting a level of at least one biomarker associated with preeclampsia in a sample from a subject involves (a) determining a level of at least one biomarker in a sample obtained from a subject, wherein the at least one biomarker is selected from the group consisting essentially of: AC073218.2, AC073218.3, ACE2, ADAMTS15, ADAMTS4, AOX1, BMP2, CTC-498J12.1, CXCL5, CXCL8, DOCK4-AS1, DSC3, GBP2, GPR126, ICAM1, IER3, IGSF10, ILIA, IL23A, INHBA, KIR2DL2, KLRF1, LINC00312, LINCO1338, LOC100506530, LOC101929174, MMP10, MT1CP, MUM1L1, NOTUM, PDGFD, PRG2, PROM1, PZP, RN7SKP16, RNASE2, RNU6-162P, RNU7-40P, RNUC-1024P, RP11-57P19.1, RP11-59H7.3, RP1-68D18.4, SAPCD1, SERPIN811, SPINK1, SULF2, TMEM27, TNC, TRPC4, and Xxbac-BPG252F; (b) determining that an absolute value of a ratio of the determined level of the biomarker in the sample to a control level of the biomarker is at least 2, thereby determining that the subject has or is at risk for preeclampsia.

In some embodiments, a method for detecting a level of at least one biomarker associated with preeclampsia in a sample from a subject involves (a) determining a level of at least one biomarker in a sample obtained from a subject, wherein the at least one biomarker is selected from the group consisting essentially of: ADAMTS8, CHI3L2, CHST7, CNR1, COCH, FBXO2, NPR1, SCARA5, and SERPINA3; and (b) determining that an absolute value of a ratio of the determined level of the biomarker in the sample to a control level of the biomarker is at least 2, thereby determining that the subject has or is at risk for preeclampsia.

In some embodiments, methods described herein further comprise determining the level of at least one additional biomarker from the group consisting essentially of: ABLIM2, ADRA2A, ANGPT2, ARHGDIB, C1Oorf10, Clorf133, C1QTNF7, C4orf49, CCDC, CCDC81, CCL8, CLIC2, CNIH3, COL14A1, COL8A1, CPE, DBC1, EDNRA, EGR1, GALNTL2, GRP, HSD17B2, IGFBP1, IGFBP5, IL1, IL15, IL1B, IRS2, ITGA11, LPAR1, LTBP1, MYCN, NCKAP5, NKAIN1, PRL and IGFBP1.

In some embodiments, a method for detecting a level of at least one biomarker associated with preeclampsia in a sample from a subject involves (a) determining a level of at least one biomarker in a sample obtained from a subject, wherein the at least one biomarker is selected from the group consisting essentially of: ADAMTS8, CHI3L2, CHST7, CNR1, COCH, FBXO2, NPR1, SCARA5, and SERPINA3; and (b) determining that an absolute value of a ratio of the determined level of the biomarker in the sample to a control level of the biomarker is less than 2, thereby determining that the subject does not have preeclampsia.

In some embodiments, methods described herein further comprise determining the level of at least one additional biomarker from the group consisting essentially of: ABLIM2, ADRA2A, ANGPT2, ARHGDIB, C1Oorf10, Clorf133, C1QTNF7, C4orf49, CCDC, CCDC81, CCL8, CLIC2, CNIH3, COL14A1, COL8A1, CPE, DBC1, EDNRA, EGR1, GALNTL2, GRP, HSD17B2, IGFBP1, IGFBP5, IL1, IL15, IL1B, IRS2, ITGA11, LPAR1, LTBP1, MYCN, NCKAP5, NKAIN1, PRL and IGFBP1.

In some embodiments, a method for detecting a level of at least one biomarker associated with preeclampsia in a sample from a subject, involves (a) determining a level of at least one biomarker in a sample obtained from a subject, wherein the at least one biomarker is selected from at least one of the following pathways: extracellular structure organization, tissue development, inflammation, immune function, transport and/or metabolism, cell signaling, transcription and/or translation, signal transduction, protein degradation, insulin related, G-protein signaling, cell cycle and activation, and unspecified; and (b) determining that an absolute value of a ratio of the determined level of the biomarker in the sample to a control level of the biomarker is at least 2, thereby determining that the subject has or is at risk for preeclampsia.

In some embodiments, a method for detecting a level of at least one biomarker associated with preeclampsia in a sample from a subject involves (a) determining a level of at least one biomarker in a sample obtained from a subject, wherein determining a level of at least one biomarker comprises a hybridization assay and at least one binding agent, and wherein the at least one binding agent is selected from the group consisting essentially of SEQ ID NOs.:1-8, and wherein the at least one biomarker is selected from the group consisting essentially of: ALDH1A1, IGFBP1, NANOS3, and HSD17B2; and (b) determining that an absolute value of a ratio of the determined level of the biomarker in the sample to a control level of the biomarker is at least 2, thereby determining that the subject has or is at risk for preeclampsia. In some embodiments, the at least one binding agent comprises at least one labeled binding agent.

In some embodiments, a method for detecting a level of at least one biomarker associated with preeclampsia in a sample from a subject involves (a) determining a level of at least one biomarker in a sample obtained from a subject, wherein determining a level of at least one biomarker comprises a hybridization assay and at least one labeled binding agent, and wherein the at least one biomarker is selected from the group consisting essentially of: CNR1, IRS2, CHST7, PRUNE2, ADAMTS8, SCARA5, SERPINA3, NPR1, LPAR1, ABLIM2, CHI3L2, LTBP1, TNFRSF8, SLC27A3, IL1, CCDC, PPAP2C, SERTADA4, COCH, FBXO2, Clorf133, and CNIH3; and (b) determining that an absolute value of a ratio of the determined level of the biomarker in the sample to a control level of the biomarker is at least 2, thereby determining that the subject has or is at risk for preeclampsia.

In some embodiments, methods described herein may further comprise treating the subject with an effective amount of an anti-preeclampsia therapy selected from the group consisting of an antihypertensive agent, an anticoagulant, a corticosteroid, an anticonvulsant, an antioxidant, a glycosaminoglycan, bed rest, hospitalization, maternal and fetal monitoring, and delivery. In some embodiments, methods described herein may further comprise treating the subject with another anti-preeclampsia therapy. In some embodiments, a subject described herein is on or has been treated with another anti-preeclampsia therapy.

In some embodiments, determining the level of a biomarker as described herein comprises performing an assay on a sample obtained from the subject.

In some embodiments, step (a) of a method described herein consists essentially of determining the level of at least five biomarkers from the group. In some embodiments, step (a) of a method described herein consists essentially of determining the level of at least seven biomarkers from the group. In some embodiments, step (a) of a method described herein consists essentially of determining the level of at least nine biomarkers from the group. In some embodiments, step (a) of a method described herein consists essentially of determining the level of at least ten biomarkers from the group. In some embodiments, step (a) of a method described herein consists essentially of determining the level of at least fifteen biomarkers from the group. In some embodiments, step (a) of a method described herein consists essentially of determining the level of all biomarkers from the group.

In some embodiments, methods described herein further consist essentially of measuring the level of PRL and IGFBP1.

In some embodiments, biomarkers consist essentially of ADAMTS8, CHI3L2, CHST7, CNR1, COCH, FBXO2, NPR1, SCARA5, and SERPINA3.

In some embodiments, determining the level of a biomarker comprises determining the level of biomarker protein. In some embodiments, the level of each biomarker protein is determined using an immunohistochemical assay, an immunoblotting assay, or a flow cytometry assay.

In some embodiments, determining the level of a biomarker comprises determining the level of biomarker nucleic acid. In some embodiments, the level of each biomarker nucleic acid is measured by a real-time reverse transcriptase PCR (RT-PCR) assay or a nucleic acid microarray assay.

In some embodiments, methods described herein further comprise transferring one or more fertilized eggs or embryos to the subject.

In some embodiments, the level of each biomarker nucleic acid is measured using a hybridization assay and at least one labeled binding agent. In some embodiments, the at least one labeled binding agent is at least one labeled oligonucleotide binding agent. In some embodiments, the at least one labeled binding agent is at least one fluorescently labeled binding agent.

In some embodiments, a sample is selected from the group consisting of a sample of endometrium tissue, endometrial stromal cells, and endometrial fluid. In some embodiments, a sample is obtained from a human. In some embodiments, a human is pregnant or is trying to become pregnant.

In some embodiments, a solid state assay device for determining the level of one or more biomarkers associated with preeclampsia, the device comprises: a chip comprising one or more analysis regions, wherein each analysis region consists essentially of a group of 5 to 129 binding partners, and wherein each of the binding partners specifically binds to an expression product of a biomarker selected from FIGS. 14-16.

In some embodiments, the solid state assay device comprises each analysis region consisting essentially of 5 to 25 binding partners from the group. In some embodiments, the solid state assay device comprises each analysis region consisting essentially of 25 to 50 binding partners from the group. In some embodiments, the solid state assay device comprises each analysis region consisting essentially of 50 to 100 binding partners from the group. In some embodiments, the solid state assay device comprises each analysis region consisting essentially of 100 to 129 binding partners from the group. In some embodiments, the solid state assay device comprises each analysis region consisting essentially of 100 to 129 binding partners from the group.

In some embodiments, the solid state assay device comprises a biomarker selected from the group consisting essentially of: ADAMTS8, CHI3L2, CHST7, CNR1, COCH, FBXO2, NPR1, SCARAS, and SERPINA3.

In some embodiments, the solid state assay device comprises a biomarker selected from the group consisting essentially of: CNR1, IRS2, CHST7, PRUNE2, ADAMTS8, SCARA5, SERPINA3, NPR1, LPAR1, ABLIM2, CHI3L2, LTBP1, TNFRSF8, SLC27A3, IL1, CCDC, PPAP2C, SERTADA4, COCH, FBXO2, Clorf133, and CNIH3.

In some embodiments, the solid state assay device comprises a biomarker selected from the group consisting essentially of: HSD17B2, ANGPT2, NCKAP5, ADRA2A, DBC1, C1QTNF7, COL8A1, EGR1, SSTR1, FBXO2, CPE, C4orf49, GRP, IGFBP5, COCH, ARHGDIB, SCG5, ITGAll, SLC35F3, RLN2, COL14A1, CLIC2, TMEM25, CCDC81, MYCN, NPR1, RASGRP2, CHI3L2, RSPO3, Cl0orf10, TMEM132C, PPAP2B, NKAIN1, ADAMTS8, IL15, SLC7A2, SERPINA3, NPTX1, CHST7, GALNTL2, SBSN, EDNRA, IL1B, SPARCL1, SCARA5, SIPA1L2, CCL8, P2RY14, CNR1, and IGFBP1.

In some embodiments, the solid state assay device comprises a biomarker selected from the group consisting essentially of: A1BG-AS1, ARL5B, BAC1-AS, C7, COL8A1, CP, CSPG4, CYP19A1, DEFB1, ENPP4, IPW, LOC101928439, LOC101929607, LOC644172, MIR365A, MIR4509-1, MIR548H1, MME-AS1, MS4A2, OGN, PRKXP1, PSMD3, RNA5SP187, RNA5SP463, RNU2-5P, RNU4-39P, RNU4-76P, RNU4ATAC1BP, RNU6-1111P, RNU6-521P, RNU6-540R, RNU6V, RNUC-901P, RP11-1026M7.3, RP11-106K3.1, RP11-12D16.2, RP11-661Al2.4, RP11-872017.8, SNORD115-32, SNORD52, SNORD71, SPINK1, TAS2R46, TRAJ59, TRBV4-2, TRIM48, TSPAN1, UGT2B7, and ZNF483.

In some embodiments, the solid state assay device comprises a biomarker selected from the group consisting essentially of: AC073218.2, AC073218.3, ACE2, ADAMTS15, ADAMTS4, AOX1, BMP2, CTC-498J12.1, CXCL5, CXCL8, DOCK4-AS1, DSC3, GBP2, GPR126, ICAM1, IER3, IGSF10, ILIA, IL23A, INHBA, KIR2DL2, KLRF1, LINC00312, LINCO1338, LOC100506530, LOC101929174, MMP10, MT1CP, MUM1L1, NOTUM, PDGFD, PRG2, PROM1, PZP, RN7SKP16, RNASE2, RNU6-162P, RNU7-40P, RNUC-1024P, RP11-57P19.1, RP11-59H7.3, RP1-68D18.4, SAPCD1, SERPIN811, SPINK1, SULF2, TMEM27, TNC, TRPC4, and Xxbac-BPG252F.

In some embodiments, the expression product of a biomarker is mRNA. In some embodiments, the expression product of a biomarker is a protein. In some embodiments, the chip is used to analyze at least one sample obtained from a subject. In some embodiments, a kit comprises the solid state assay device and instructions for use.

These and other aspects of the technology are illustrated by the following non-limiting drawings, and described in more detail in the detailed description and examples.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1A shows representative immunofluorescent images of localization of F-actin by rhodamine-phalloidin staining of hESCs from women with uncomplicated pregnancies (no preeclampsia (PE)). Scale bar =100 p.m.

FIG. 1B shows representative immunofluorescent images of localization of F-actin by rhodamine-phalloidin staining of hESCs from women who had sPE.

FIG. 1C shows a graph of PRL levels detected in conditioned medium of non-decidualized hESCs and decidualized hESCs from normal pregnancy patients.

FIG. 1D shows a graph of PRL levels detected in conditioned medium of non-decidualized hESCs and decidualized hESCs from sPE patients.

FIG. 1E shows a graph summarizing PRL levels in conditioned medium of non-decidualized hESCs and decidualized hESCs from normal pregnancy and sPE patients. **p<0.01, ***p<0.005. n.s., non-significant. Scale bar=100 μm.

FIG. 1F shows a graph of IGFBP1 levels detected in conditioned medium of non-decidualized hESCs and decidualized hESCs from normal pregnancy patients.

FIG. 1G shows a graph of IGFBP1 levels detected in conditioned medium of non-decidualized hESCs and decidualized hESCs from sPE patients.

FIG. 1H shows a graph summarizing the IGFBP1 levels in conditioned medium of non-decidualized hESCs and decidualized hESCs from normal pregnancy and sPE patients. **p<0.01, ***p<0.005. n.s., non-significant.

FIG. 2A shows a schematic drawing of the study design. hESCs were isolated from endometrial biopsies and a portion of the cells were decidualized in vitro. The donors were non-pregnant women with previous normal pregnancy outcomes or former sPE patients.

FIG. 2B shows a summary of the LIMMA paired-comparisons showing the number of differentially expressed genes (DEGs) by >2- fold between the groups.

FIG. 2C shows a heat map of the 5 DEGs that were modulated prior to decidualization of hESCs from the normal pregnancy outcome group and previous sPE patients.

FIG. 2D shows a heat map of the 50 most highly DEGs (total=74; see also FIG. 13) that were modulated during decidualization of hESCs from donors who had normal pregnancy outcomes.

FIG. 2E shows a heat map of the 50 most highly DEGs (total=129; see FIG. 14) that were misexpressed following decidualization of hESCs from donors with a former sPE pregnancy as compared to those with normal pregnancies. * denotes mRNA expression patterns validated by qRT- PCR; A, fold change.

FIG. 3A shows a schematic drawing of the study design. Laser microdissection enabled isolation of portions of the decidua basalis from the basal plate and decidua parietalis, (adjacent to the fetal membranes).

FIG. 3B shows a summary of the LIMMA paired-comparisons showing the number of differentially expressed genes (DEGs) between equivalent decidual compartments in sPE vs. preterm birth with no signs of infection (noninfected preterm birth; nPTB).

FIG. 3C shows a heat map showing the 50 most highly DEGs (total=79; see also FIG. 15) in the decidua basalis of nPTB vs. sPE patients.

FIG. 3D shows a heat map showing the 50 most highly DEGs (total=227; see also FIG. 16) in the decidua parietalis of nPTB vs. sPE patients.

FIGS. 4A-4D show representative tissue sections of the maternal-fetal interface that contained portions of the decidua basalis or the decidua parietalis that were co-immunostained with an antibody against cytokeratin (CK7), which enabled visualization of cytotrophoblasts (CTBs), and decidual markers PRL (FIGS. 4A-4B show sections from the decidua basalis and decidua parietalis, respectively) and IGFBP1 (FIGS. 4C-4D show sections from the decidua basalis and decidua parietalis, respectively).

FIGS. 4E-4F show representative adjacent sections from the decidua basalis (FIG. 4E) and decidua parietalis (FIG. 4F) stained with anti-vimentin (VIM) to label DEC cells. Nuclei were visualized with DAPI. Representative areas (3-4) of each sample were analyzed (sPE, n=5 cases; nPTB, n=4 cases). iCTBs, invasive CTBs; Am, amnion; schCTBs, smooth chorion CTBs. Scale bars: 100 μm.

FIGS. 4G-4H show graphs of relative PRL immunoreactivity (FIG. 4G) and relative IGFBP1 immunoreactivity (FIG. 4H) in the decidua basalis and decidua parietalis of noninfected preterm birth (nPTB) and sPE patients.

FIGS. 5A-5J show representative images demonstrating that freshly isolated stromal cells from decidual biopsies of sPE patients displayed decidualization defects in culture. Cells were isolated from either the decidua basalis or the decidua parietalis and analyzed at P0. Donors were women whose pregnancies were complicated by preterm birth with no signs of infection (noninfected preterm birth, nPTB; n=4) or severe preeclampsia (sPE; n=5).

FIGS. 5A-5B show representative immunofluorescent images of cells from the decidua basalis or the decidua parietalis in which the F-actin cytoskeleton of the cells was stained by rhodamine-phalloidin staining and nuclei were stained with DAPI. In nPTB pregnancies, cells from either decidual compartment had a polygonal shape with a complex well-developed network of actin filaments. In contrast, the cells from sPE pregnancies were flattened with a much less well developed actin cytoskeleton.

FIGS. 5C-5H show representative immunofluorescent images of cells from the decidua basalis or the decidua parietalis in nPTB or sPE patients stained for prolactin (PRL) (FIGS. 5C-5D), insulin-like growth factor binding protein 1 (IGFBP1) (FIGS. 5E-5F), and vimentin (FIGS. 5G-5H).

FIGS. 5I-5J show graphs of PRL (FIG. 5I) and IGFBP1 secretion (FIG. 5J) from the decidua basalis or the decidua parietalis in nPTB or sPE patients. Data are the mean±SEM of each sample, which was analyzed in triplicate. *p<0.05, **p<0.01, ***p<0.001; scale bars, 100 μm.

FIGS. 6A-6B show representative immunofluorescent images of hESCs of decidualized or non-decidualized biopsies from the decidua basalis or the decidua parietalis of nPTB patients (FIG. 6A) and sPE patients (FIG. 6B). The F-actin cytoskeleton was stained via rhodamine-phalloidin staining. Nuclei were stained with DAPI.

FIGS. 6C-6D show graphs of PRL (FIG. 6C) and IGFBP1 (FIG. 6D) secretion in decidualized and non-decidualized biopsies from the decidua basalis or the decidua parietalis. Levels were measured using ELISA. Data are the mean±SEM of each sample, which was analyzed in triplicate. *p<0.05, **p<0.01; n.s., not significant; scale bar, 100 μm.

FIG. 7A shows a diagram of the experimental design. Decidual cells were isolated from the basalis (DB) or parietalis (DP) and cultured overnight. Then the conditioned medium (CM) was isolated. The donors were either women whose pregnancies were complicated by preterm birth with no signs of infection (noninfected preterm birth, nPTB; n=3) or by severe preeclampsia (sPE; n=4). CTBs were isolated from second trimester placentas (15-17 wks, n=4; 18-20 wks, n=3; 21-23 wks, n=3). They were cultured (72 h) on Matrigel-coated Transwell filters in medium conditioned by the nPTB or sPE decidual cells. CTBs and cellular processes that reached the undersides of the filters were counted.

FIG. 7B shows graphs of the numbers of CTBs and cellular processes in cultures from nPTB donors and sPE donors. As compared to the equivalent nPTB samples, CM from the cells of sPE donors significantly inhibited CTB invasion regardless of whether they were isolated from the DB or DP.

FIG. 7C shows graphs of the numbers of CTBs and cellular processes in the presence of PRL and IGFBP1 (10 ng/ml each) in cultures from nPTB donors and sPE donors. The addition of PRL and IGFBP1 to fresh medium restored CTB invasion to the levels that were observed when the cells were incubated in CM from nPTB cultures. Data are expressed as the mean±SEM of duplicate wells. **p<0.01, ***p<0.001; n.s., not significant.

FIG. 8A shows results from the Ingenuity Pathway Analysis of the data from control samples described in FIGS. 2A-2E and FIG. 13.

FIG. 8B shows results from Ingenuity Pathway Analysis of the data from the severe preeclampsia samples described in FIGS. 3A-3B and FIG. 14. Black bars denote up regulated pathways, grey bars denote down regulated pathways.

FIG. 8C shows a diagram of overlapping genes that were up regulated during in vitro decidualization of cells from women who had normal pregnancy outcomes and were down regulated during in vitro decidualization of cells from women with a previous sPE pregnancy.

FIG. 8D shows a diagram of overlapping genes that were down regulated during in vitro decidualization of cells from women who had normal pregnancy outcomes and were up regulated during in vitro decidualization of cells from women with a previous sPE pregnancy.

FIG. 9 shows a graph of mRNA expression data obtained by qRT-PCR validation of the microarray data. Fold changes were calculated as gene expression levels of sPE vs. control human endometrial stromal cell samples that were decidualized in culture. FC, fold change

FIG. 10 shows results of a pathway analysis of genes that were dysregulated in the decidua parietalis 876 samples (sPE vs. nPTB). The data were generated by Ingenuity Pathway Analysis of the results described in FIG. 3D and FIG. 16. Black bars, up regulated; grey bars, down regulated. p<0.05.

FIGS. 11A-11C show representative images of tissue sections of the analyzed maternal-fetal interface containing portions of the decidua parietalis and the smooth chorion. The donors were either women who had preterm birth with no signs of infection (nPTB; n=5) or severe preeclampsia (sPE; n=5). The tissue sections were co-immunostained with an antibody against cytokeratin (CK7), which enabled visualization of cytotrophoblasts (CTBs), and antibodies that recognized proteins encoded by genes that were differentially expressed in the decidua parietalis of donors with severe preeclampsia: PEG1 (MEST) (FIG. 11A), PRG2 (FIG. 11B), and BMP2 (FIG. 11C). Nuclei were stained with DAPI. Relative to nPTB samples, PEG1 and PRG2 were up regulated in sPE; BMP2 was down regulated. Scale bar, 100 μm.

FIG. 12A shows a graph of PRL levels in medium conditioned by isolated stromal cells from samples of the decidua basalis and the decidua parietalis of sPE (n=4) or control nPTB (n=3) cases measured using ELISA.

FIG. 12B shows a graph of IGPBP1 levels in medium conditioned by isolated stromal cells from samples of the decidua basalis and the decidua parietalis of sPE (n=4) or control nPTB (n=3) cases measured using ELISA.

FIG. 13 shows a heatmap listing the genes that were differentially expressed by 2-fold or greater during in vitro decidualization of control human endometrial stromal cells. The fold changes are shown on the roght (Δ).

FIG. 14 shows a heatmap listing the genes that were differentially expressed by 2-fold or greater during in vitro decidualization of human endometrial stromal cells isolated from former sPE patients. * =genes whose expression patterns were validated by qRT-PCR. The fold changes are shown on the roght (Δ).

FIG. 15 shows a heatmap listing the genes that were differentially expressed by 2-fold or greater in decidual basalis samples isolated from sPE patients compared to patients having preterm birth with no signs of infection (noninfected preterm birth; nPTB). The fold changes are shown on the roght (Δ).

FIG. 16 shows a heatmap listing the genes that were differentially expressed by 2-fold or greater in decidual parietalis samples isolated from sPE patients compared to patients having preterm birth with no signs of infection (noninfected preterm birth; nPTB). The fold changes are shown on the roght (Δ).

FIGS. 17A-17C show that an endometrial transcriptional profile corroborates in vivo a decidualization defect in sPE patients. Principal component analysis (PCA) showing a distribution of samples based on global (FIG. 17A) and targeted (FIG. 17B) RNA-seq approaches. FIG. 17C shows the correlation between the gene expression of the 129 genes targeted by guided sequencing and the same genes identified by global RNA-seq.

FIG. 18 provides the DIFFERENTIAL GENE EXPRESSION PANEL of in vitro decidualized human endometrial stromal cells (hESCs) isolated from former severe preeclampsia patients compared with normal pregnant women as described in Example 8. Gene expression values were pre-processed (half-background median intensity values were subtracted from the average intensity of each spot), normalized and analyzed using bioconductor LIMMA package in the R software. The significant differentially expressed genes were determined by statistical analysis of false discovery rate (adjusted p-value).

### DETAILED DESCRIPTION OF THE INVENTION

Aspects of the present disclosure relate to methods and compositions for detecting differentially expressed genes. In some embodiments, differentially expressed genes are detected in a sample from a subject (e.g., a patient) having or at risk for preeclampsia. Such methods may be useful for clinical purposes, for example, identifying a subject (e.g., a patient) having or at risk for preeclampsia, selecting a treatment, monitoring preeclampsia progression, assessing the efficacy of a treatment against preeclampsia, or determining a course of treatment for a subject (e.g., a patient). The assay methods described herein may also be useful for non-clinical applications, for example, for research purposes, including, e.g., studying the mechanism of preeclampsia development and/or biological pathways and/or biological processes involved in preeclampsia, and developing new therapies for preeclampsia based on such studies.

### Biomarkers

Methods described herein are based, at least in part, on the identification of biomarkers that were found to be differentially present in women that had preeclampsia (PE) in a previous pregnancy compared to women that had a normal pregnancy.

As used herein, the term “biomarker” or “biomarker set” refers to a biological molecule (e.g., a protein) or set of such biological molecules that are present at specific levels. One or more such biomarkers may be present in a specific population of cells (e.g., human endometrial stromal cells (hESCs)) and the level of each biomarker may deviate from the level of the same biomarker in a different population of cells and/or in a different subject (e.g., patient). For example, a biomarker that is indicative of preeclampsia may have an elevated level or a reduced level in a sample from a subject (e.g., a sample from a subject that has or is at risk for preeclampsia) relative to the level of the same marker in a control sample (e.g., a sample from a normal subject, such as a subject who does not have or is not at risk for preeclampsia).

Exemplary biomarkers indicative of preeclampsia are provided in Table 1. In some embodiments, a biomarker is differentially expressed in a sample from a subject that had preeclampsia in a previous pregnancy compared to a sample from a subject that had a normal pregnancy. In some embodiments, a biomarker is differentially expressed in a sample that has been decidualized compared to a sample that is non-decidualized.

TABLE 1
Exemplary biomarkers.

| | | | Chromosome |
|---|---|---|---|
| Gene Symbol | Description | HGNC ID* | location |
| AADAC | arylacetamide deacetylase (esterase) | HGNC: 17 | 3q25.1 |
| ABLIM2 | actin binding LIM protein family, member 2 | HGNC: 19195 | 4p16.1 |
| ADAMTS19 | ADAM metallopeptidase with thrombospondin type 1 motif, 19 | HGNC: 17111 | 5q23.3 |
| ADAMTS8 | ADAM metallopeptidase with thrombospondin type 1 motif, 8 | HGNC: 224 | 11q24.3 |
| ADRA2A | adrenergic, alpha-2A-, receptor | HGNC: 281 | 10q25.2 |
| ALDH1A1 | aldehyde dehydrogenase 1 family, member A1 | HGNC: 402 | 9q21.13 |
| ANGPT2 | angiopoietin 2 | HGNC: 485 | 8p23.1 |
| ANXA2 | annexin A2 | HGNC: 537 | 15q22.2 |
| ARHGDIB | Rho GDP dissociation inhibitor (GDI) beta | HGNC: 679 | 12p12.3 |
| ATCAY | ataxia, cerebellar, Cayman type (caytaxin) | HGNC: 779 | 19p13.3 |
| BAIAP2L2 | BAI1-associated protein 2-like 2 | HGNC: 26203 | 22q13.1 |
| BDNF | brain-derived neurotrophic factor | HGNC: 1033 | 11p14.1 |
| C10orf10 | chromosome 10 open reading frame 10 | HGNC: 23355 | 10q11.21 |
| C14orf37 | chromosome 14 open reading frame 37 | HGNC: 19846 | 14q23.1 |
| C17orf107 | chromosome 17 open reading frame 107 | HGNC: 37238 | 17p13.2 |
| C1orf133 | chromosome 1 open reading frame 133 | HGNC: 32019 | 1q32.2 |
| C1QTNF7 | C1q and tumor necrosis factor related protein 7 | HGNC: 14342 | 4p15.32 |
| C4orf49 | mitochondria-localized glutamic acid-rich protein (MGARP) | HGNC: 29969 | 4q31.1 |
| C6orf176 | long intergenic non-protein coding RNA 473 | HGNC: 21160 | 6q27 |
| CA12 | carbonic anhydrase XII | HGNC: 1371 | 15q22.2 |
| CCDC81 | coiled-coil domain containing 81 | HGNC: 26281 | 11q14.2 |
| CCL8 | chemokine (C-C motif) ligand 8 | HGNC: 10635 | 17q12 |
| CFD | complement factor D (adipsin) | HGNC: 2771 | 19p13.3 |
| CHI3L2 | chitinase 3-like 2 | HGNC: 1933 | 1p13.2 |
| CHODL | chondrolectin | HGNC: 17807 | 21q21.1 |
| CHST7 | carbohydrate (N-acetylglucosamine 6-O) sulfotransferase 7 | HGNC: 13817 | Xp11.3 |
| CLEC3B | C-type lectin domain family 3, member B | HGNC: 11891 | 3p21.31 |
| CLIC3 | chloride intracellular channel 3 | HGNC: 2064 | 9q34.3 |
| CNIH3 | cornichon homolog 3 | HGNC: 26802 | 1q42.12 |
| CNR1 | cannabinoid receptor 1 (brain) | HGNC: 2159 | 6q15 |
| COCH | coagulation factor C homolog, cochlin (Limulus polyphemus) | HGNC: 2180 | 14q12 |
| COL14A1 | collagen, type XIV, alpha 1 | HGNC: 2191 | 8q24.12 |
| COL15A1 | collagen, type XV, alpha 1 | HGNC: 2192 | 9q22.33 |
| COL8A1 | collagen, type VIII, alpha 1 | HGNC: 2215 | 3q12.1 |
| CPE | carboxypeptidase E | HGNC: 2303 | 4q32.3 |
| CRLF1 | cytokine receptor-like factor 1 | HGNC: 2364 | 19p12 |
| DBC1 | deleted in bladder cancer 1 | HGNC: 2687 | 9q33.1 |
| DCN | decorin | HGNC: 2705 | 12q21.33 |
| DDIT4 | DNA-damage-inducible transcript 4 | HGNC: 24944 | 10q22.1 |
| DENND2A | DENN/MADD domain containing 2A | HGNC: 22212 | 7q34 |
| DES | desmin | HGNC: 2770 | 2q35 |
| DMKN | dermokine | HGNC: 25063 | 19q13.12 |
| DUSP6 | dual specificity phosphatase 6 | HGNC: 3072 | 12q21.33 |
| EDNRA | endothelin receptor type A | HGNC: 3179 | 4q31.22-q31.23 |
| EDNRB | endothelin receptor type B | HGNC: 3180 | 13q22.3 |
| EFEMP1 | EGF-containing fibulin-like extracellular matrix protein 1 | HGNC: 3218 | 2p16.1 |
| EGR1 | early growth response 1 | HGNC: 3238 | 5q31.2 |
| EHD3 | EH-domain containing 3 | HGNC: 3244 | 2p23.1 |
| KAZALD1 | Kazal-type serine peptidase inhibitor domain 1 | | 5q15 |
| PLIN2 | perilipin 2 (PLIN2), transcript variant 2, non-coding RNA. | | |
| ERAP2 | endoplasmic reticulum aminopeptidase 2 | HGNC: 29499 | |
| ERP27 | endoplasmic reticulum protein 27 | HGNC: 26495 | 12p12.3 |
| F2RL2 | coagulation factor II (thrombin) receptor-like 2 | HGNC: 3539 | 5q13.3 |
| FAM19A2 | family with sequence similarity 19 (chemokine), member A2 | HGNC: 21589 | 12q14.1 |
| FAM38B | family with sequence similarity 38, member B | HGNC: 26270 | 18p11.22-p11.21 |
| FAT1 | FAT tumor suppressor homolog | HGNC: 3595 | 4q35.2 |
| FBXO2 | F-box protein 2 | HGNC: 13581 | 1p36.22 |
| FST | follistatin | HGNC: 3971 | 5q11.2 |
| GAL | galanin | HGNC: 4114 | 11q13.2 |
| GALNT14 | UDP-N-acetyl-alpha-D-galactosamine | HGNC: 22946 | 2p23.1 |
| GALNTL2 | N-acetylgalactosaminyltransferase-like 2 | HGNC: 21531 | 3p25.1 |
| GBP2 | guanylate binding protein 2, interferon-inducible | HGNC: 4183 | 1p22.2 |
| GGT5 | gamma-glutamyltransferase 5 | HGNC: 4260 | 22q11.23 |
| GRP | gastrin-releasing peptide | HGNC: 4605 | 18q21.32 |
| HMCN1 | hemicentin 1 | HGNC: 19194 | 1q25.3-q31.1 |
| HSD17B2 | hydroxysteroid (17-beta) dehydrogenase 2 | HGNC: 5211 | 16q23.3 |
| IGFBP1 | insulin-like growth factor binding protein 1 | HGNC: 5469 | 7p12.3 |
| IGFBP5 | insulin-like growth factor binding protein 5 | HGNC: 5474 | 2q35 |
| IL15 | interleukin 15 | HGNC: 5977 | 4q31.21 |
| IL1B | interleukin 1, beta | HGNC: 5992 | 2q14.1 |
| IRS2 | insulin receptor substrate 2 | HGNC: 6126 | 13q34 |
| ISM1 | isthmin 1 homolog | HGNC: 16213 | 20p12.1 |
| ITGA11 | integrin, alpha 11 | HGNC: 6136 | 15q23 |
| KCNJ8 | potassium inwardly-rectifying channel, subfamily J, member 8 | HGNC: 6269 | 12p12.1 |
| KLF2 | Kruppel-like factor 2 | HGNC: 6347 | 19p13.11 |
| KRTAP17-1 | keratin associated protein 17-1 | HGNC: 18917 | 17q21.2 |
| LAMA5 | laminin, alpha 5 | HGNC: 6485 | 20q13.33 |
| NLRP1 | uncharacterized LOC728392 | | |
| LOXL4 | lysyl oxidase-like 4 | HGNC: 17171 | 10q24.2 |
| LPAR1 | lysophosphatidic acid receptor 1 | HGNC: 3166 | 9q31.3 |
| LPL | lipoprotein lipase | HGNC: 6677 | 8p21.3 |
| LRRC15 | leucine rich repeat containing 15 | HGNC: 20818 | 3q29 |
| LSAMP | limbic system-associated membrane protein | HGNC: 6705 | 3q13.31 |
| LTBP1 | latent transforming growth factor beta binding protein 1 | HGNC: 6714 | 2p22.3 |
| LYPD1 | LY6/PLAUR domain containing 1 | HGNC: 28431 | 2q21.2 |
| MEST | mesoderm specific transcript homolog | HGNC: 7028 | 7q32.2 |
| MFAP2 | microfibrillar-associated protein 2 | HGNC: 7033 | 1p36.13 |
| MRVI1 | murine retrovirus integration site 1 homolog | HGNC: 7237 | 11p15.4 |
| MYCN | v-myc myelocytomatosis viral related oncogene | HGNC: 7559 | 2p24.3 |
| MYLK | myosin, light chain kinase | HGNC: 7590 | 3q21.1 |
| NANOS3 | nanos homolog 3 | HGNC: 22048 | 19p13.13 |
| NCKAP5 | NCK-associated protein 5 | HGNC: 29847 | 2q21.2 |
| NKAIN1 | Na+/K+ transporting ATPase interacting 1 | HGNC: 25743 | 1p35.2 |
| NPR1 | natriuretic peptide receptor A/guanylate cyclase A | HGNC: 7943 | 1q21.3 |
| NPTX1 | neuronal pentraxin I | HGNC: 7952 | 17q25.3 |
| OLFML1 | olfactomedin-like 1 | HGNC: 24473 | 11p15.4 |
| OXTR | oxytocin receptor | HGNC: 8529 | 3p25.3 |
| P2RY14 | purinergic receptor P2Y, G-protein coupled, 14 | HGNC: 16442 | 3q25.1 |
| PDGFD | platelet derived growth factor D | HGNC: 30620 | 11q22.3 |
| PITX1 | paired-like homeodomain transcription factor 1 | HGNC: 9004 | 5q31.1 |
| PPAP2B | phosphatidic acid phosphatase type 2B | HGNC: 9229 | 1p32.2 |
| PRUNE2 | prune homolog 2 | HGNC: 25209 | 9q21.2 |
| RASGRP2 | RAS guanyl releasing protein 2 (calcium and DAG-regulated) | HGNC: 9879 | 11q13.1 |
| RASL11B | RAS-like, family 11, member B | HGNC: 23804 | 4q12 |
| REEP2 | receptor accessory protein 2 | HGNC: 17975 | 5q31.2 |
| RGS16 | regulator of G-protein signalling 16 | HGNC: 9997 | 1q25.3 |
| RGS20 | regulator of G-protein signalling 20 | HGNC: 14600 | 8q11.23 |
| RHOU | ras homolog gene family, member U | HGNC: 17794 | 1q42.13 |
| RLN2 | relaxin 2 | HGNC: 10027 | 9p24.1 |
| RSPO3 | R-spondin 3 homolog | HGNC: 20866 | 6q22.33 |
| SBSN | suprabasin | HGNC: 24950 | 19q13.13 |
| SCARA5 | scavenger receptor class A, member 5 (putative) | HGNC: 28701 | 8p21.1 |
| SCG5 | secretogranin V (7B2 protein) | HGNC: 10816 | 15q13.3 |
| SERPINA3 | serpin peptidase inhibitor, clade A member 3 | HGNC: 16 | 14q32.13 |
| SERTAD4 | SERTA domain containing 4 | HGNC: 25236 | 1q32.2 |
| SIPA1L2 | signal-induced proliferation-associated 1 like 2 | HGNC: 23800 | 1q42.2 |
| SLC35F3 | solute carrier family 35, member F3 | HGNC: 23616 | 1q42.2 |
| SLC7A2 | solute carrier family 7, member 2 | HGNC: 11060 | 8p22 |
| SLITRK6 | SLIT and NTRK-like family, member 6 | HGNC: 23503 | 13q31.1 |
| SPARCL1 | SPARC-like 1 (mast9, hevin) | HGNC: 11220 | 4q22.1 |
| SSTR1 | somatostatin receptor 1 | HGNC: 11330 | 14q13 |
| SULF1 | sulfatase 1 | HGNC: 20391 | 8q13.2-q13.3 |
| TMEM132C | transmembrane protein 132C | HGNC: 25436 | 12q24.32 |
| TMEM25 | transmembrane protein 25 | HGNC: 25890 | 11q23.3 |
| TNFAIP6 | tumor necrosis factor, alpha-induced protein 6 | HGNC: 11898 | 2q23.3 |
| TNFRSF10C | tumor necrosis factor receptor superfamily, member 10c | HGNC: 11906 | 8p21.3 |
| TNFRSF8 | tumor necrosis factor receptor superfamily, member 8 | HGNC: 11923 | 1p36.22 |
| TTR | transthyretin (prealbumin, amyloidosis type I) | HGNC: 12405 | 18q12.1 |
| WNT6 | wingless-type MMTV integration site family, member 6 | HGNC: 12785 | 2q35 |

*HGNC—HUGO Gene Nomenclature Committee gene identification number

In still other embodiments, the biomarkers are one or more (e.g., all or substantially all) of those defined in Table A. In some embodiments, the biomarkers represent a set of 36 differentially expressed genes (“DEGs”) from biological samples taken from patients with prior severe pre-eclampsia (sPE) compared to control biological tissues taken from term and pre-term patients not having sPE. In various embodiments, the biological samples are endometrial samples, which may comprise endometrial tissue, endometrial cells, and/or endometrial fluids. In other embodiments, the biological sample can be blood. Table A biomarkers include:

TABLE A
Global RNAseq: sPE vs. Control

| | | | | | | | RefSeq |
|---|---|---|---|---|---|---|---|
| | | | | | | | peptide |
| | | | | | | | ID or |
| | | | | | | RefSeq | other |
| | HGNC | logFC (log 2 of | | P-Value | | mRNA | sequence |
| Biomarker | Symbol | Fold Change) | P-Value | adjusted | Gene name | ID | ID |
| ARSI | ARSI | −3.587605505 | 2.11681E−08 | 0.000387079 | arylsulfatase | NM_001012301 | NP_001012301 |
| | | | | | family, | | |
| | | | | | member I | | |
| | | | | | [Source:HGNC | | |
| | | | | | Symbol; | | |
| | | | | | Acc:32521] | | |
| BEX1 | BEX1 | −2.774461726 | 1.3E−06 | 0.023771878 | brain | NM_018476 | NP_060946 |
| | | | | | expressed, | | |
| | | | | | X-linked 1 | | |
| | | | | | [Source:HGNC | | |
| | | | | | Symbol; | | |
| | | | | | Acc:1036] | | |
| CBLN1 | CBLN1 | −3.81081001 | 5.69807E−08 | 0.001041949 | cerebellin 1 | NM_004352 | NP_004343 |
| | | | | | precursor | | |
| | | | | | [Source:HGNC | | |
| | | | | | Symbol; | | |
| | | | | | Acc:1543] | | |
| CDH2 | CDH2 | −1.61895692 | 1.98353E−06 | 0.036270814 | cadherin 2, | NM_001792 | NP_001783 |
| | | | | | type 1, N- | | |
| | | | | | cadherin | | |
| | | | | | (neuronal) | | |
| | | | | | [Source:HGNC | | |
| | | | | | Symbol; | | |
| | | | | | Acc:1759] | | |
| CNTNAP2 | CNTNAP2 | −3.945441911 | 5.44354E−08 | 0.000995406 | contactin | NM_014141 | NP_054860 |
| | | | | | associated | | |
| | | | | | protein-like 2 | | |
| | | | | | [Source:HGNC | | |
| | | | | | Symbol; | | |
| | | | | | Acc:13830] | | |
| ECEL1 | ECEL1 | −3.470449867 | 1.77862E−07 | 0.003252381 | endothelin | NM_004826 | NP_004817 |
| | | | | | converting | | |
| | | | | | enzyme-like 1 | | |
| | | | | | [Source:HGNC | | |
| | | | | | Symbol; | | |
| | | | | | Acc:3147] | | |
| EMC10 | EMC10 | −0.671118696 | 1.19156E−06 | 0.021788824 | ER | NM_175063 | NP_996261 |
| | | | | | membrane | | |
| | | | | | protein | | |
| | | | | | complex | | |
| | | | | | subunit 10 | | |
| | | | | | [Source:HGNC | | |
| | | | | | Symbol; | | |
| | | | | | Acc:27609] | | |
| ENC1 | ENC1 | −1.80048072 | 2.04901E−06 | 0.037468176 | ectodermal- | NM_003633 | NP_001243504 |
| | | | | | neural | | |
| | | | | | cortex 1 | | |
| | | | | | (with BTB | | |
| | | | | | domain) | | |
| | | | | | [Source:HGNC | | |
| | | | | | Symbol; | | |
| | | | | | Acc:3345] | | |
| FBP1 | FBP1 | −1.549431749 | 1.27959E−08 | 0.000233986 | fructose-1,6- | NM_000507 | NP_000498 |
| | | | | | bisphosphatase 1 | | |
| | | | | | [Source:HGNC | | |
| | | | | | Symbol; | | |
| | | | | | Acc:3606] | | |
| FJX1 | FJX1 | −2.374272697 | 3.73038E−08 | 0.000682138 | four jointed | NM_014344 | NP_055159 |
| | | | | | box 1 | | |
| | | | | | (Drosophila) | | |
| | | | | | [Source:HGNC | | |
| | | | | | Symbol; | | |
| | | | | | Acc:17166] | | |
| GABRP | GABRP | 1.181340791 | 1.35574E−06 | 0.024791075 | gamma- | NM_014211 | NP_055026 |
| | | | | | aminobutyric | | |
| | | | | | acid | | |
| | | | | | (GABA) A | | |
| | | | | | receptor, pi | | |
| | | | | | [Source:HGNC | | |
| | | | | | Symbol; | | |
| | | | | | Acc:4089] | | |
| IGSF11 | IGSF11 | 1.683998765 | 9.2268E−07 | 0.016872132 | immunoglobulin | NM_152538 | NP_001015887 |
| | | | | | superfamily, | | |
| | | | | | member 11 | | |
| | | | | | [Source:HGNC | | |
| | | | | | Symbol; | | |
| | | | | | Acc:16669] | | |
| ITGAll | ITGAll | −2.736802153 | 2.15071E−06 | 0.039327904 | integrin, | NM_001004439 | NP_001004439 |
| | | | | | alpha 11 | | |
| | | | | | [Source:HGNC | | |
| | | | | | Symbol; | | |
| | | | | | Acc:6136] | | |
| KCNF1 | KCNF1 | −3.752006045 | 1.85958E−08 | 0.000340043 | potassium | NM_002236 | NP_002227 |
| | | | | | voltage- | | |
| | | | | | gated | | |
| | | | | | channel, | | |
| | | | | | subfamily F, | | |
| | | | | | member 1 | | |
| | | | | | [Source:HGNC | | |
| | | | | | Symbol; | | |
| | | | | | Acc:6246] | | |
| KCNN4 | KCNN4 | −2.651719862 | 1.01836E−06 | 0.018621748 | potassium | NM_002250 | NP_002241 |
| | | | | | intermediate/ | | |
| | | | | | small | | |
| | | | | | conductance | | |
| | | | | | calcium- | | |
| | | | | | activated | | |
| | | | | | channel, | | |
| | | | | | subfamily | | |
| | | | | | N, member 4 | | |
| | | | | | [Source:HGNC | | |
| | | | | | Symbol; | | |
| | | | | | Acc:6293] | | |
| LAMA1 | LAMA1 | −1.870143613 | 1.12664E−06 | 0.020601707 | laminin, | NM_005559 | NP_005550 |
| | | | | | alpha 1 | | |
| | | | | | [Source:HGNC | | |
| | | | | | Symbol; | | |
| | | | | | Acc:6481] | | |
| LAMPS | LAMPS | −2.716066638 | 1.72607E−07 | 0.003156293 | lysosomal- | NM_012261 | NP_001186826 |
| | | | | | associated | | |
| | | | | | membrane | | |
| | | | | | protein | | |
| | | | | | family, | | |
| | | | | | member 5 | | |
| | | | | | [Source:HGNC | | |
| | | | | | Symbol; | | |
| | | | | | Acc:16097] | | |
| MMP11 | MMP11 | −4.049111102 | 9.95777E−10 | 1.82088E−05 | matrix | NM_005940 | NP_005931 |
| | | | | | metallopeptidase 11 | | |
| | | | | | (stromelysin 3) | | |
| | | | | | [Source:HGNC | | |
| | | | | | Symbol; | | |
| | | | | | Acc:7157] | | |
| MTND1P23 | MTND1P23 | 4.573551937 | 4.42476E−07 | 0.008091118 | Homo | NG_032769.1 | |
| | | | | | sapiens MT- | | |
| | | | | | ND1 | | |
| | | | | | pseudogene 23 | | |
| | | | | | (MTND1P23) on | | |
| | | | | | chromosome 1. | | |
| NKD1 | NKD1 | −2.480372911 | 3.93988E−07 | 0.007204472 | naked | NM_033119 | NP_149110 |
| | | | | | cuticle | | |
| | | | | | homolog 1 | | |
| | | | | | (Drosophila) | | |
| | | | | | [Source:HGNC | | |
| | | | | | Symbol; | | |
| | | | | | Acc:17045] | | |
| OGDHL | OGDHL | −1.907799107 | 2.06701E−06 | 0.03779726 | oxoglutarate | NM_018245 | NP_060715 |
| | | | | | dehydrogena | | |
| | | | | | se-like | | |
| | | | | | [Source:HGNC | | |
| | | | | | Symbol; | | |
| | | | | | Acc:25590] | | |
| PRKXP1 | PRKXP1 | 1.598996743 | 5.38435E−08 | 0.000984582 | Homo | NR_073405.1 | |
| | | | | | sapiens | | |
| | | | | | PRKX | | |
| | | | | | pseudogene 1 | | |
| | | | | | (PRKXP1), | | |
| | | | | | non-coding | | |
| | | | | | RNA | | |
| RAB3B | RAB3B | −2.507182089 | 4.40427E−09 | 8.05365E−05 | RAB3B, | NM_002867 | NP_002858 |
| | | | | | member | | |
| | | | | | RAS | | |
| | | | | | oncogene | | |
| | | | | | family | | |
| | | | | | [Source:HGNC | | |
| | | | | | Symbol; | | |
| | | | | | Acc:9778] | | |
| REEP2 | REEP2 | −1.805521999 | 8.62192E−07 | 0.015766045 | receptor | NM_001271803 | NP_057690 |
| | | | | | accessory | | |
| | | | | | protein 2 | | |
| | | | | | [Source:HGNC | | |
| | | | | | Symbol; | | |
| | | | | | Acc:17975] | | |
| RGS6 | RGS6 | 1.909427023 | 8.47058E−07 | 0.015489299 | regulator of | NM_001204424 | NP_001191353 |
| | | | | | G-protein | | |
| | | | | | signaling 6 | | |
| | | | | | [Source:HGNC | | |
| | | | | | Symbol; | | |
| | | | | | Acc:10002] | | |
| RIMBP2 | RIMBP2 | 1.80076761 | 2.39661E−06 | 0.043824324 | RIMS | NM_015347 | NP_056162 |
| | | | | | binding | | |
| | | | | | protein 2 | | |
| | | | | | [Source:HGNC | | |
| | | | | | Symbol; | | |
| | | | | | Acc:30339] | | |
| RIMS4 | RIMS4 | −4.512193347 | 4.51452E−08 | 0.000825525 | regulating | NM_001205317 | NP_001192246 |
| | | | | | synaptic | | |
| | | | | | membrane | | |
| | | | | | exocytosis 4 | | |
| | | | | | [Source:HGNC | | |
| | | | | | Symbol; | | |
| | | | | | Acc:16183] | | |
| RP11- | RP11- | 2.192753573 | 5.54735E−07 | 0.01014388 | Transcript | ENSG00000236740.2 | |
| 411K7.1 | 411K7.1 | | | | | | |
| RP11- | RP11- | 1.401210324 | 2.04852E−06 | 0.037459286 | Transcript | ENST00000602585 | |
| 52612.5 | 52612.5 | | | | | | |
| RPS6KA5 | RPS6KA5 | 1.300281806 | 3.99892E−07 | 0.00731243 | ribosomal | NM_004755 | NP_004746 |
| | | | | | protein S6 | | |
| | | | | | kinase, | | |
| | | | | | 90 kDa, | | |
| | | | | | polypeptide 5 | | |
| | | | | | [Source:HGNC | | |
| | | | | | Symbol; | | |
| | | | | | Acc:10434] | | |
| SBK1 | SBK1 | −2.137185787 | 1.80378E−06 | 0.032983937 | 5H3 domain | NM_001024401 | NP_001019572 |
| | | | | | binding | | |
| | | | | | kinase 1 | | |
| | | | | | [Source:HGNC | | |
| | | | | | Symbol; | | |
| | | | | | Acc:17699] | | |
| SLC47A1 | SLC47A1 | −3.651257909 | 6.15591E−07 | 0.011256698 | solute | NM_018242 | NP_060712 |
| | | | | | carrier | | |
| | | | | | family 47 | | |
| | | | | | (multidrug | | |
| | | | | | and toxin | | |
| | | | | | extrusion), | | |
| | | | | | member 1 | | |
| | | | | | [Source:HGNC | | |
| | | | | | Symbol; | | |
| | | | | | Acc:25588] | | |
| TMEM215 | TMEM215 | −4.365091644 | 1.27141E−06 | 0.023249083 | transmembrane | NM_212558 | NP_997723 |
| | | | | | protein 215 | | |
| | | | | | [Source:HGNC | | |
| | | | | | Symbol; | | |
| | | | | | Acc:33816] | | |
| TMSB15A | TMSB15A | −2.245261944 | 3.42605E−08 | 0.000626487 | thymosin | NM_021992 | NP_068832 |
| | | | | | beta 15a | | |
| | | | | | [Source:HGNC | | |
| | | | | | Symbol; | | |
| | | | | | Acc:30744] | | |
| UCN2 | UCN2 | −2.690533892 | 8.50616E−07 | 0.015554369 | urocortin 2 | NM_033199 | NP_149976 |
| | | | | | [Source:HGNC | | |
| | | | | | Symbol; | | |
| | | | | | Acc:18414] | | |
| ZNF471 | ZNF471 | 0.855670032 | 2.71592E−07 | 0.004966338 | zinc finger | NM_020813 | NP_065864 |
| | | | | | protein 471 | | |
| | | | | | [Source:HGNC | | |
| | | | | | Symbol; | | |
| | | | | | Acc:23226] | | |

In other embodiments, the biomarkers are one or more (e.g., all or substantially all) of those defined in Table B. In some embodiments, the biomarkers represent a set of 246 differentially expressed genes (“DEGs”) from biological samples taken from patients with prior severe pre-eclampsia (sPE) compared to control biological tissues taken from pre-term patients not having sPE (e.g., the pre-term patients have the same gestational age as the pre-eclampsia patients). In various embodiments, the biological samples are endometrial samples, which may comprise endometrial tissue, endometrial cells, and/or endometrial fluids. In other embodiments, the biological sample can be blood. Table B biomarkers include:

TABLE B
Global RNAseq: sPE vs. Control Preterm

| | logFC (log 2 | | | | RefSeq mRNA |
|---|---|---|---|---|---|
| | of Fold | | P-Value | | ID or other |
| Gene list | Change) | P-Value | adjusted | Gene name | sequence ID |
| ACOT8 | 0.69187535 | 1.197E−06 | 0.02207636 | acyl-CoA thioesterase 8 | NM_005469 |
| | | | | [Source: HGNC | |
| | | | | Symbol; Acc: 15919] | |
| ADAMTS15 | 2.68534827 | 3.48366E−08 | 0.000642491 | ADAM metallopeptidase | NM_139055 |
| | | | | with thrombospondin type 1 | |
| | | | | motif, 15 [Source: HGNC | |
| | | | | Symbol; Acc: 16305] | |
| ADCYAP1R1 | −3.538652029 | 6.67774E−09 | 0.000123158 | adenylate cyclase activating | NM_001199636 |
| | | | | polypeptide 1 (pituitary) | |
| | | | | receptor type I | |
| | | | | [Source: HGNC | |
| | | | | Symbol; Acc: 242] | |
| ADRA2A | 2.702442333 | 1.89483E−11 | 3.49464E−07 | adrenoceptor alpha 2A | NM_000681 |
| | | | | [Source: HGNC | |
| | | | | Symbol; Acc: 281] | |
| ADRA2B | −4.517710381 | 7.32143E−08 | 0.001350291 | adrenoceptor alpha 2B | NM_000682 |
| | | | | [Source: HGNC | |
| | | | | Symbol; Acc: 282] | |
| AF064858.6 | 3.435198389 | 2.79154E−07 | 0.005148431 | | |
| AIMP1 | 1.652897317 | 2.34605E−10 | 4.32682E−06 | aminoacyl tRNA synthetase | NM_004757 |
| | | | | complex-interacting | |
| | | | | multifunctional protein 1 | |
| | | | | [Source: HGNC | |
| | | | | Symbol; Acc: 10648] | |
| ANKRD55 | 5.509476886 | 9.32958E−10 | 1.72065E−05 | ankyrin repeat domain 55 | NM_024669 |
| | | | | [Source: HGNC | |
| | | | | Symbol; Acc: 25681] | |
| AOX1 | 4.516807178 | 3.11756E−10 | 5.74972E−06 | aldehyde oxidase 1 | NM_001159 |
| | | | | [Source: HGNC | |
| | | | | Symbol; Acc: 553] | |
| AR | −1.215581871 | 1.21273E−06 | 0.022366412 | androgen receptor | NM_000044 |
| | | | | [Source: HGNC | |
| | | | | Symbol; Acc: 644] | |
| ASIC2 | −5.028153925 | 6.00364E−07 | 0.01107252 | acid-sensing (proton-gated) | NM_183377 |
| | | | | ion channel 2 | |
| | | | | [Source: HGNC | |
| | | | | Symbol; Acc: 99] | |
| ASTL | −2.992369379 | 1.79344E−06 | 0.033076466 | astacin-like | NM_001002036 |
| | | | | metalloendopeptidase | |
| | | | | (M12 family) | |
| | | | | [Source: HGNC | |
| | | | | Symbol; Acc: 31704] | |
| ATP1B1 | −1.745411437 | 6.58518E−09 | 0.000121451 | ATPase, Na+/K+ | NM_001677 |
| | | | | transporting, beta 1 | |
| | | | | polypeptide [Source: HGNC | |
| | | | | Symbol; Acc: 804] | |
| ATP6V1A | 1.418591732 | 1.16396E−06 | 0.021466903 | ATPase, H+ transporting, | NM_001690 |
| | | | | lysosomal 70 kDa, V1 | |
| | | | | subunit A [Source: HGNC | |
| | | | | Symbol; Acc: 851] | |
| ATP8B3 | −2.041703248 | 2.01468E−09 | 3.71568E−05 | ATPase, aminophospholipid | NM_138813 |
| | | | | transporter, class I, type 8B, | |
| | | | | member 3 [Source: HGNC | |
| | | | | Symbol; Acc: 13535] | |
| B4GALNT2 | 5.390551493 | 4.2253E−12 | 7.79272E−08 | beta-1,4-N-acetyl-galactosaminyl | NM_001159387 |
| | | | | transferase 2 | |
| | | | | [Source: HGNC | |
| | | | | Symbol; Acc: 24136] | |
| BBC3 | −1.899797914 | 1.41123E−06 | 0.026027328 | BCL2 binding component 3 | NM_001127240 |
| | | | | [Source: HGNC | |
| | | | | Symbol; Acc: 17868] | |
| BCMO1 | 2.271619985 | 7.03248E−07 | 0.01297001 | beta-carotene | NM_017429 |
| | | | | 15,15′-monooxygenase 1 | |
| | | | | [Source: HGNC | |
| | | | | Symbol; Acc: 13815] | |
| BMF | −1.875641672 | 1.48552E−06 | 0.027397489 | Bcl2 modifying factor | NM_001003940 |
| | | | | [Source: HGNC | |
| | | | | Symbol; Acc: 24132] | |
| BMP3 | −3.804208762 | 1.22942E−06 | 0.022674156 | bone morphogenetic protein | NM_001201 |
| | | | | 3 [Source: HGNC | |
| | | | | Symbol; Acc: 1070] | |
| BMPR1B | −1.578853259 | 1.63307E−14 | 3.01187E−10 | bone morphogenetic protein | NM_001203 |
| | | | | receptor, type IB | |
| | | | | [Source: HGNC | |
| | | | | Symbol; Acc: 1077] | |
| BNC2 | −1.251377909 | 6.43269E−07 | 0.011863802 | basonuclin 2 [Source: HGNC | NM_017637 |
| | | | | Symbol; Acc: 30988] | |
| C10orf82 | −2.985591618 | 7.25644E−08 | 0.001338305 | chromosome 10 open | NM_144661 |
| | | | | reading frame 82 | |
| | | | | [Source: HGNC | |
| | | | | Symbol; Acc: 28500] | |
| C11orf54 | 0.714827324 | 4.01543E−07 | 0.00740566 | chromosome 11 open | NM_014039 |
| | | | | reading frame 54 | |
| | | | | [Source: HGNC | |
| | | | | Symbol; Acc: 30204] | |
| C1orf168 | −1.995574957 | 5.33751E−08 | 0.000984397 | chromosome 1 open reading | NM_001004303 |
| | | | | frame 168 [Source: HGNC | |
| | | | | Symbol; Acc: 27295] | |
| C1R | 1.259302641 | 1.54107E−06 | 0.028421917 | complement component 1, r | NM_001733 |
| | | | | subcomponent | |
| | | | | [Source: HGNC | |
| | | | | Symbol; Acc: 1246] | |
| C6orf141 | 1.735722062 | 7.39534E−07 | 0.013639228 | chromosome 6 open reading | NM_001145652 |
| | | | | frame 141 [Source: HGNC | |
| | | | | Symbol; Acc: 21351] | |
| CACHD1 | −1.046365664 | 2.94929E−10 | 5.43938E−06 | cache domain containing 1 | NM_020925 |
| | | | | [Source: HGNC | |
| | | | | Symbol; Acc: 29314] | |
| CADM1 | −1.337554867 | 6.38837E−07 | 0.011782065 | cell adhesion molecule 1 | NM_014333 |
| | | | | [Source: HGNC | |
| | | | | Symbol; Acc: 5951] | |
| CAPN8 | 2.762380519 | 1.644E−06 | 0.030320204 | calpain 8 [Source: HGNC | NM_001143962 |
| | | | | Symbol; Acc: 1485] | |
| CASC15 | −1.148652957 | 1.47746E−06 | 0.027248834 | cancer susceptibility | |
| | | | | candidate 15 (non-protein | |
| | | | | coding) [Source: HGNC | |
| | | | | Symbol; Acc: 28245] | |
| CBLN1 | −4.010110105 | 9.15665E−08 | 0.00168876 | cerebellin 1 precursor | NM_004352 |
| | | | | [Source: HGNC | |
| | | | | Symbol; Acc: 1543] | |
| CCL20 | −5.113195654 | 5.63314E−07 | 0.010389195 | chemokine (C-C motif) | NM_004591 |
| | | | | ligand 20 [Source: HGNC | |
| | | | | Symbol; Acc: 10619] | |
| CCNA1 | −2.385434422 | 6.1319E−07 | 0.011309055 | cyclin A1 [Source: HGNC | NM_003914 |
| | | | | Symbol; Acc: 1577] | |
| CD83 | −1.975460115 | 6.14586E−07 | 0.011334808 | CD83 molecule | NM_001040280 |
| | | | | [Source: HGNC | |
| | | | | Symbol; Acc: 1703] | |
| CDYL2 | 2.378806089 | 1.11622E−10 | 2.05865E−06 | chromodomain protein, | NM_152342 |
| | | | | Y-like 2 [Source: HGNC | |
| | | | | Symbol; Acc: 23030] | |
| CITED2 | 1.514230155 | 7.15851E−07 | 0.013202434 | Cbp/p300-interacting | NM_006079 |
| | | | | transactivator, with | |
| | | | | Glu/Asp-rich | |
| | | | | carboxy-terminal | |
| | | | | domain, 2 | |
| | | | | [Source: HGNC | |
| | | | | Symbol; Acc: 1987] | |
| CLIC5 | −2.435490269 | 4.44483E−10 | 8.19761E−06 | chloride intracellular | NM_016929 |
| | | | | channel 5 [Source: HGNC | |
| | | | | Symbol; Acc: 13517] | |
| CNPPD1 | 0.710086104 | 2.58887E−06 | 0.047746525 | cyclin Pas1/PHO80 domain | NM_015680 |
| | | | | containing 1 [Source: HGNC | |
| | | | | Symbol; Acc: 25220] | |
| CNTNAP2 | −4.299586327 | 7.26267E−10 | 1.33945E−05 | contactin associated protein-like | NM_014141 |
| | | | | 2 [Source: HGNC | |
| | | | | Symbol; Acc: 13830] | |
| COL27A1 | −1.39117313 | 2.10431E−06 | 0.03880975 | collagen, type XXVII, alpha | NM_032888 |
| | | | | 1 [Source: HGNC | |
| | | | | Symbol; Acc: 22986] | |
| COLEC11 | 1.908329795 | 2.5719E−08 | 0.000474335 | collectin sub-family member | NM_199235 |
| | | | | 11 [Source: HGNC | |
| | | | | Symbol; Acc: 17213] | |
| COLEC12 | −1.799745002 | 4.88981E−09 | 9.01827E−05 | collectin sub-family member | NM_130386 |
| | | | | 12 [Source: HGNC | |
| | | | | Symbol; Acc: 16016] | |
| CPA3 | −4.621109613 | 1.59415E−09 | 2.9401E−05 | carboxypeptidase A3 (mast | NM_001870 |
| | | | | cell) [Source: HGNC | |
| | | | | Symbol; Acc: 2298] | |
| CRISPLD1 | −2.127988293 | 2.54522E−07 | 0.004694147 | cysteine-rich secretory | NM_031461 |
| | | | | protein LCCL domain | |
| | | | | containing 1 [Source: HGNC | |
| | | | | Symbol; Acc: 18206] | |
| CSF3 | −8.528069184 | 6.14499E−08 | 0.001133321 | colony stimulating factor 3 | NM_172219 |
| | | | | (granulocyte) | |
| | | | | [Source: HGNC | |
| | | | | Symbol; Acc: 2438] | |
| CTD-2055G21.1 | 3.434967804 | 4.91262E−09 | 9.06034E−05 | | |
| CTD-2308N23.2 | 3.906778447 | 9.37434E−07 | 0.017289086 | | |
| CXADR | −1.081718202 | 6.71873E−08 | 0.001239136 | coxsackie virus and | NM_001338 |
| | | | | adenovirus receptor | |
| | | | | [Source: HGNC | |
| | | | | Symbol; Acc: 2559] | |
| CXCL14 | 4.720735911 | 9.04311E−08 | 0.001667821 | chemokine (C-X-C motif) | NM_004887 |
| | | | | ligand 14 [Source: HGNC | |
| | | | | Symbol; Acc: 10640] | |
| CXCL2 | −3.919068288 | 6.8187E−07 | 0.012575729 | chemokine (C-X-C motif) | NM_002089 |
| | | | | ligand 2 [Source: HGNC | |
| | | | | Symbol; Acc: 4603] | |
| CXCL3 | −4.525507281 | 1.58776E−08 | 0.000292831 | chemokine (C-X-C motif) | NM_002090 |
| | | | | ligand 3 [Source: HGNC | |
| | | | | Symbol; Acc: 4604] | |
| CYP26B1 | −2.408502194 | 1.62761E−06 | 0.030017961 | cytochrome P450, family | NM_019885 |
| | | | | 26, subfamily B, | |
| | | | | polypeptide 1 | |
| | | | | [Source: HGNC | |
| | | | | Symbol; Acc: 20581] | |
| DACT2 | −2.49897268 | 7.06156E−11 | 1.30236E−06 | dishevelled-binding | NM_001286351 |
| | | | | antagonist of beta-catenin 2 | |
| | | | | [Source: HGNC | |
| | | | | Symbol; Acc: 21231] | |
| DCAF12L1 | −1.361552608 | 1.18737E−06 | 0.021898739 | DDB1 and CUL4 associated | NM_178470 |
| | | | | factor 12-like 1 | |
| | | | | [Source: HGNC | |
| | | | | Symbol; Acc: 29395] | |
| DERA | 0.859368078 | 1.96803E−06 | 0.036296438 | deoxyribose-phosphate | NM_015954 |
| | | | | aldolase (putative) | |
| | | | | [Source: HGNC | |
| | | | | Symbol; Acc: 24269] | |
| DIO2 | −2.19245741 | 7.78027E−07 | 0.01434915 | deiodinase, iodothyronine, | NM_013989 |
| | | | | type II [Source: HGNC | |
| | | | | Symbol; Acc: 2884] | |
| DNAJC6 | 2.174208329 | 9.97437E−07 | 0.018395728 | DnaJ (Hsp40) homolog, | NM_014787 |
| | | | | subfamily C, member 6 | |
| | | | | [Source: HGNC | |
| | | | | Symbol; Acc: 15469] | |
| DOK7 | −1.92516382 | 2.5423E−07 | 0.004688761 | docking protein 7 | NM_001164673 |
| | | | | [Source: HGNC | |
| | | | | Symbol; Acc: 26594] | |
| DPP4 | 3.401796215 | 6.2767E−08 | 0.001157611 | dipeptidyl-peptidase 4 | NM_001935 |
| | | | | [Source: HGNC | |
| | | | | Symbol; Acc: 3009] | |
| DUSP2 | −2.786037801 | 9.04487E−10 | 1.66814E−05 | dual specificity phosphatase | NM_004418 |
| | | | | 2 [Source: HGNC | |
| | | | | Symbol; Acc: 3068] | |
| ECEL1 | −3.430906831 | 2.39757E−06 | 0.044218346 | endothelin converting | NM_004826 |
| | | | | enzyme-like 1 | |
| | | | | [Source: HGNC | |
| | | | | Symbol; Acc: 3147] | |
| EDN2 | −4.791636649 | 4.98182E−07 | 0.009187973 | endothelin 2 [Source: HGNC | NM_001956 |
| | | | | Symbol; Acc: 3177] | |
| EGR2 | −2.169703771 | 1.33473E−06 | 0.024616497 | early growth response 2 | NM_001136177 |
| | | | | [Source: HGNC | |
| | | | | Symbol; Acc: 3239 | |
| EGR3 | −2.386691723 | 7.94632E−08 | 0.00146554 | early growth response 3 | NM_004430 |
| | | | | [Source: HGNC | |
| | | | | Symbol; Acc: 3240] | |
| EIF4E3 | 1.364347149 | 1.31169E−07 | 0.002419152 | eukaryotic translation | NM_001134651 |
| | | | | initiation factor 4E family | |
| | | | | member 3 [Source: HGNC | |
| | | | | Symbol; Acc: 31837] | |
| EMILIN2 | 1.721937278 | 1.08603E−07 | 0.002002971 | elastin microftbril interfacer | NM_032048 |
| | | | | 2 [Source: HGNC | |
| | | | | Symbol; Acc: 19881] | |
| ENCI | −2.020894347 | 8.11265E−08 | 0.001496215 | ectodermal-neural cortex 1 | NM_003633 |
| | | | | (with BTB domain) | |
| | | | | [Source: HGNC | |
| | | | | Symbol; Acc: 3345] | |
| EPHA7 | −2.367547512 | 6.05299E−07 | 0.011163531 | EPH receptor A7 | NM_004440 |
| | | | | [Source: HGNC | |
| | | | | Symbol; Acc: 3390] | |
| EYA2 | −1.219898529 | 1.94998E−06 | 0.035963426 | eyes absent homolog 2 | NM_005244 |
| | | | | (Drosophila) [Source: HGNC | |
| | | | | Symbol; Acc: 3520] | |
| FAM149A | 1.417028943 | 2.00678E−07 | 0.0037011 | family with sequence | NM_015398 |
| | | | | similarity 149, member A | |
| | | | | [Source: HGNC | |
| | | | | Symbol; Acc: 24527] | |
| FAM169A | −1.283285127 | 6.85029E−07 | 0.012633986 | family with sequence | NM_015566 |
| | | | | similarity 169, member A | |
| | | | | [Source: HGNC | |
| | | | | Symbol; Acc: 29138] | |
| FAM222A | −2.363372191 | 1.01173E−07 | 0.001865936 | family with sequence | NM_032829 |
| | | | | similarity 222, member A | |
| | | | | [Source: HGNC | |
| | | | | Symbol; Acc: 25915] | |
| FILIP1 | 2.403675423 | 7.36808E−08 | 0.001358895 | filamin A interacting protein | NM_015687 |
| | | | | 1 [Source: HGNC | |
| | | | | Symbol; Acc: 21015] | |
| FLRT1 | −2.183317357 | 1.50345E−06 | 0.027728084 | fibronectin leucine rich | NM_013280 |
| | | | | transmembrane protein 1 | |
| | | | | [Source: HGNC | |
| | | | | Symbol; Acc: 3760] | |
| FNDC4 | 1.265152667 | 1.71816E−06 | 0.031687985 | fibronectin type III domain | NM_022823 |
| | | | | containing 4 [Source: HGNC | |
| | | | | Symbol; Acc: 20239] | |
| GALNT5 | −5.270598358 | 4.01775E−09 | 7.40994E−05 | UDP-N-acetyl-alpha-D- | NM_014568 |
| | | | | galactosamine:polypeptide | |
| | | | | N-acetylgalactosaminyltransferase | |
| | | | | 5 (GalNAc-T5) | |
| | | | | [Source: HGNC | |
| | | | | Symbol; Acc: 4127] | |
| GDPD1 | −1.628631078 | 6.22026E−07 | 0.011472017 | glycerophosphodiester | NM_182569 |
| | | | | phosphodiesterase domain | |
| | | | | containing 1 [Source: HGNC | |
| | | | | Symbol; Acc: 20883] | |
| GJB1 | 1.622486549 | 4.61256E−07 | 0.008506943 | gap junction protein, beta 1, | NM_001097642 |
| | | | | 32 kDa [Source: HGNC | |
| | | | | Symbol; Acc: 4283] | |
| GJB2 | −3.061759674 | 1.88599E−07 | 0.003478327 | gap junction protein, beta 2, | NM_004004 |
| | | | | 26 kDa [Source: HGNC | |
| | | | | Symbol; Acc: 4284] | |
| GJB3 | −3.164117254 | 9.90009E−11 | 1.82587E−06 | gap junction protein, beta 3, | NM_024009 |
| | | | | 31 kDa [Source: HGNC | |
| | | | | Symbol; Acc: 4285] | |
| GPBAR1 | 2.948826733 | 1.39817E−08 | 0.000257865 | G protein-coupled bile acid | NM_001077191 |
| | | | | receptor 1 [Source: HGNC | |
| | | | | Symbol; Acc: 19680] | |
| GPR125 | −0.752744803 | 1.00777E−07 | 0.001858634 | G protein-coupled receptor | NM_145290 |
| | | | | 125 [Source: HGNC | |
| | | | | Symbol; Acc: 13839] | |
| GRIP1 | −0.998250704 | 2.20363E−06 | 0.040641585 | glutamate receptor | NM_021150 |
| | | | | interacting protein 1 | |
| | | | | [Source: HGNC | |
| | | | | Symbol; Acc: 18708] | |
| GSG1L | 5.948087652 | 8.48561E−09 | 0.0001565 | GSG1-like [Source: HGNC | NM_001109763 |
| | | | | Symbol; Acc: 28283] | |
| HBA2 | −5.149582422 | 3.34394E−07 | 0.006167226 | hemoglobin, alpha 2 | NM_000517 |
| | | | | [Source: HGNC | |
| | | | | Symbol; Acc: 4824] | |
| HBB | −5.145307485 | 1.62456E−06 | 0.029961697 | hemoglobin, beta | NM_000518 |
| | | | | [Source: HGNC | |
| | | | | Symbol; Acc: 4827] | |
| HMCN2 | −2.237365112 | 7.15405E−07 | 0.013194217 | hemicentin 2 | UniProtKB - |
| | | | | [Source: HGNC | Q8NDA2 |
| | | | | Symbol; Acc: 21293] | |
| HMGA2 | −2.785892757 | 1.42334E−15 | 2.62507E−11 | high mobility group AT-hook | NM_003483 |
| | | | | 2 [Source: HGNC | |
| | | | | Symbol; Acc: 5009] | |
| HNF1A-AS1 | 2.166201607 | 9.53629E−07 | 0.017587784 | HNF1A antisense RNA 1 | HGNC: 26785 |
| | | | | [Source: HGNC | |
| | | | | Symbol; Acc: 26785] | |
| HSPB6 | 1.752248031 | 3.5787E−07 | 0.006600198 | heat shock protein, | NM_144617 |
| | | | | alpha-crystallin-related, | |
| | | | | B6 [Source: HGNC | |
| | | | | Symbol; Acc: 26511] | |
| HTR1D | 2.502606525 | 9.87129E−08 | 0.001820561 | 5-hydroxytryptamine | NM_000864 |
| | | | | (serotonin) receptor 1D, G | |
| | | | | protein-coupled | |
| | | | | [Source: HGNC | |
| | | | | Symbol; Acc: 5289] | |
| ICAM1 | −2.56444072 | 1.44284E−06 | 0.026610219 | intercellular adhesion | NM_000201 |
| | | | | molecule 1 [Source: HGNC | |
| | | | | Symbol; Acc: 5344] | |
| IGFN1 | −4.649700123 | 9.42512E−12 | 1.73827E−07 | immunoglobulin-like and | NM_001164586 |
| | | | | fibronectin type III domain | |
| | | | | containing 1 [Source: HGNC | |
| | | | | Symbol; Acc: 24607] | |
| IGHA2 | −3.518781346 | 6.07919E−07 | 0.011211855 | immunoglobulin heavy | HGNC: 5479 |
| | | | | constant alpha 2 (A2m | |
| | | | | marker) [Source: HGNC | |
| | | | | Symbol; Acc: 5479] | |
| IGHG1 | −6.300201954 | 2.29659E−10 | 4.2356E−06 | immunoglobulin heavy | HGNC: 5525 |
| | | | | constant gamma 1 (G1m | |
| | | | | marker) [Source: HGNC | |
| | | | | Symbol; Acc: 5525] | |
| IGHG3 | −5.166486611 | 3.36723E−09 | 6.21018E−05 | immunoglobulin heavy | HGNC: 5527 |
| | | | | constant gamma 3 (G3m | |
| | | | | marker) [Source: HGNC | |
| | | | | Symbol; Acc: 5527] | |
| IGLC2 | −5.917021991 | 1.41308E−07 | 0.002606146 | immunoglobulin lambda | HGNC: 5856 |
| | | | | constant 2 (Kern-Oz-marker) | |
| | | | | [Source: HGNC | |
| | | | | Symbol; Acc: 5856] | |
| IL17RB | −1.548484433 | 4.2342E−08 | 0.000780913 | interleukin 17 receptor B | NM_018725 |
| | | | | [Source: HGNC | |
| | | | | Symbol; Acc: 18015] | |
| IL1RN | −2.999581957 | 1.4155E−06 | 0.026106035 | interleukin 1 receptor | NM_173843 |
| | | | | antagonist [Source: HGNC | |
| | | | | Symbol; Acc: 6000] | |
| IL6ST | 1.591849882 | 1.25785E−06 | 0.023198501 | interleukin 6 signal | NM_175767 |
| | | | | transducer (gp130, | |
| | | | | oncostatin M receptor) | |
| | | | | [Source: HGNC | |
| | | | | Symbol; Acc: 6021] | |
| IL7R | −3.043764316 | 2.70884E−08 | 0.000499591 | interleukin 7 receptor | NM_002185 |
| | | | | [Source: HGNC | |
| | | | | Symbol; Acc: 6024] | |
| IL8 | −5.490122699 | 2.08611E−09 | 3.84741E−05 | interleukin 8 [Source: HGNC | NM_000584 |
| | | | | Symbol; Acc: 6025] | |
| INPP5J | −1.653165645 | 1.82659E−06 | 0.033687863 | inositol | NM_001284285 |
| | | | | polyphosphate-5-phosphatase | |
| | | | | J [Source: HGNC | |
| | | | | Symbol; Acc: 8956] | |
| ITGA11 | −3.214895692 | 1.5359E−08 | 0.000283266 | integrin, alpha 11 | NM_001004439 |
| | | | | [Source: HGNC | |
| | | | | Symbol; Acc: 6136] | |
| ITGB6 | −3.610290921 | 4.6583E−08 | 0.000859131 | integrin, beta 6 | NM_001282388 |
| | | | | [Source: HGNC | |
| | | | | Symbol; Acc: 6161] | |
| KB-1615E4.2 | 5.472871578 | 2.26307E−10 | 4.17378E−06 | | |
| KB-1615E4.3 | 4.157388201 | 1.0063E−08 | 0.000185592 | | |
| KCNF1 | −3.761366436 | 3.80811E−07 | 0.007023297 | potassium voltage-gated | NM_002236 |
| | | | | channel, subfamily F, | |
| | | | | member 1 [Source: HGNC | |
| | | | | Symbol; Acc: 6246] | |
| KCNN4 | −2.891656531 | 8.94898E−08 | 0.00165046 | potassium | NM_002250 |
| | | | | intermediate/small | |
| | | | | conductance calcium- | |
| | | | | activated channel, subfamily | |
| | | | | N, member 4 | |
| | | | | [Source: HGNC | |
| | | | | Symbol; Acc: 6293] | |
| KLF10 | −1.441307382 | 2.95199E−07 | 0.005444353 | Kruppel-like factor 10 | NM_005655 |
| | | | | [Source: HGNC | |
| | | | | Symbol; Acc: 11810] | |
| KLK2 | −2.790665511 | 4.45311E−07 | 0.008212869 | kallikrein-related peptidase | NM_001002231 |
| | | | | 2 [Source: HGNC | |
| | | | | Symbol; Acc: 6363] | |
| L3MBTL3 | −0.850949226 | 2.29586E−07 | 0.004234255 | 1(3)mbt-like 3 (Drosophila) | NM_001007102 |
| | | | | [Source: HGNC | |
| | | | | Symbol; Acc: 23035] | |
| LACTB2 | 1.5392927 | 6.22613E−07 | 0.011482849 | lactamase, beta 2 | NM_016027 |
| | | | | [Source: HGNC | |
| | | | | Symbol; Acc: 18512] | |
| LAMA3 | −1.358023561 | 1.21649E−07 | 0.002243567 | laminin, alpha 3 | NM_198129 |
| | | | | [Source: HGNC | |
| | | | | Symbol; Acc: 6483] | |
| LAMP5 | −2.709451458 | 4.26487E−07 | 0.007865708 | lysosomal-associated | NM_001199897 |
| | | | | membrane protein family, | |
| | | | | member 5 [Source: HGNC | |
| | | | | Symbol; Acc: 16097] | |
| LDHD | 1.60712676 | 5.21563E−10 | 9.61919E−06 | lactate dehydrogenase D | NM_153486 |
| | | | | [Source: HGNC | |
| | | | | Symbol; Acc: 19708] | |
| LIPC | 2.580463423 | 3.52424E−08 | 0.000649976 | lipase, hepatic | NM_000236 |
| | | | | [Source: HGNC | |
| | | | | Symbol; Acc: 6619] | |
| LMCD1 | 1.828321156 | 1.80345E−07 | 0.0033261 | LIM and cysteine-rich | NM_014583 |
| | | | | domains 1 [Source: HGNC | |
| | | | | Symbol; Acc: 6633] | |
| LRFN4 | 1.093023496 | 7.15094E−09 | 0.000131885 | leucine rich repeat and | NM_024036 |
| | | | | fibronectin type III domain | |
| | | | | containing 4 [Source: HGNC | |
| | | | | Symbol; Acc: 28456] | |
| LRRN1 | −2.881914599 | 1.42339E−07 | 0.002625157 | leucine rich repeat neuronal | NM_020873 |
| | | | | 1 [Source: HGNC | |
| | | | | Symbol; Acc: 20980] | |
| LTBP1 | −1.638879412 | 2.6752E−08 | 0.000493386 | latent transforming growth | NM_001166265 |
| | | | | factor beta binding protein 1 | |
| | | | | [Source: HGNC | |
| | | | | Symbol; Acc: 6714] | |
| LYPLAL1 | 1.953734845 | 4.82225E−08 | 0.000889368 | lysophospholipase-like 1 | NM_138794 |
| | | | | [Source: HGNC | |
| | | | | Symbol; Acc: 20440] | |
| MANSC4 | 4.358837557 | 3.69825E−13 | 6.82068E−09 | MANSC domain containing | NM_001146221 |
| | | | | 4 [Source: HGNC | |
| | | | | Symbol; Acc: 40023] | |
| MAOB | 1.513169186 | 1.29086E−07 | 0.002380739 | monoamine oxidase B | NM_000898 |
| | | | | [Source: HGNC | |
| | | | | Symbol; Acc: 6834] | |
| MAP2 | −1.785267097 | 2.67576E−06 | 0.049348997 | microtubule-associated | NM_001039538 |
| | | | | protein 2 [Source: HGNC | |
| | | | | Symbol; Acc: 6839] | |
| MBOAT4 | 3.662102638 | 9.42192E−07 | 0.017376848 | membrane bound | NM_001100916 |
| | | | | O-acyltransferase domain | |
| | | | | containing 4 [Source: HGNC | |
| | | | | Symbol; Acc: 32311] | |
| MCM7 | −0.811066478 | 4.59548E−08 | 0.000847545 | minichromosome | NM_001278595 |
| | | | | maintenance complex | |
| | | | | component 7 | |
| | | | | [Source: HGNC | |
| | | | | Symbol; Acc: 6950] | |
| MECOM | −1.13698389 | 4.04232E−09 | 7.45525E−05 | MDS1 and EVI1 complex | NM_001164000 |
| | | | | locus [Source: HGNC | |
| | | | | Symbol; Acc: 3498] | |
| MEG8 | 1.678095609 | 9.31761E−07 | 0.017184476 | maternally expressed 8 | NR_003080 |
| | | | | (non-protein coding) | |
| | | | | [Source: HGNC | |
| | | | | Symbol; Acc: 14574] | |
| MEG9 | 1.947221767 | 5.62055E−08 | 0.001036599 | maternally expressed 9 | HGNC: 43874 |
| | | | | (non-protein coding) | |
| | | | | [Source: HGNC | |
| | | | | Symbol; Acc: 43874] | |
| MIR5572 | 3.955201272 | 1.02179E−08 | 0.000188449 | microRNA 5572 | |
| | | | | [Source: HGNC | |
| | | | | Symbol; Acc: 43476] | |
| MMP7 | −5.130884656 | 2.78829E−08 | 0.000514244 | matrix metallopeptidase 7 | NM_002423 |
| | | | | (matrilysin, uterine) | |
| | | | | [Source: HGNC | |
| | | | | Symbol; Acc: 7174] | |
| MRPS2 | 1.371536726 | 2.31142E−07 | 0.004262958 | mitochondrial ribosomal | NM_016034 |
| | | | | protein S2 [Source: HGNC | |
| | | | | Symbol; Acc: 14495] | |
| MSX2 | −2.20135656 | 4.53789E−09 | 8.36924E−05 | msh homeobox 2 | NM_002449 |
| | | | | [Source: HGNC | |
| | | | | Symbol; Acc: 7392] | |
| MT1L | 2.701242101 | 2.173E−06 | 0.040076626 | metallothionein 1L | HGNC: 43476 |
| | | | | (gene/pseudogene) | |
| | | | | [Source: HGNC | |
| | | | | Symbol; Acc: 7404] | |
| MTF1 | 1.819627868 | 5.64964E−08 | 0.001041963 | metal-regulatory | NM_005955 |
| | | | | transcription factor 1 | |
| | | | | [Source: HGNC | |
| | | | | Symbol; Acc: 7428] | |
| NEDD9 | −1.883766165 | 8.43086E−13 | 1.5549E−08 | neural precursor cell | NM_001271033 |
| | | | | expressed, developmentally | |
| | | | | down-regulated 9 | |
| | | | | [Source: HGNC | |
| | | | | Symbol; Acc: 7733] | |
| NEO1 | −0.944060538 | 2.12372E−07 | 0.00391678 | neogenin 1 [Source: HGNC | NM_002499 |
| | | | | Symbol; Acc: 7754] | |
| NKD1 | −2.802277986 | 7.2213E−08 | 0.001331824 | naked cuticle homolog 1 | NM_033119 |
| | | | | (Drosophila) [Source: HGNC | |
| | | | | Symbol; Acc: 17045] | |
| NPTX2 | −2.01802093 | 3.15364E−07 | 0.005816253 | neuronal pentraxin II | NM_002523 |
| | | | | [Source: HGNC | |
| | | | | Symbol; Acc: 7953] | |
| NRG1 | −3.335929366 | 1.37297E−08 | 0.000253217 | neuregulin 1 [Source: HGNC | NM_001159995 |
| | | | | Symbol; Acc: 7997] | |
| NTN1 | −1.772153813 | 1.59935E−08 | 0.000294968 | netrin 1 [Source: HGNC | NM_004822 |
| | | | | Symbol; Acc: 8029] | |
| OVGP1 | −2.908471261 | 2.21514E−06 | 0.04085378 | oviductal glycoprotein 1, | NM_002557 |
| | | | | 120 kDa [Source: HGNC | |
| | | | | Symbol; Acc: 8524] | |
| PC | 1.816746047 | 4.2574E−08 | 0.000785193 | pyruvate carboxylase | NM_022172 |
| | | | | [Source: HGNC | |
| | | | | Symbol; Acc: 8636] | |
| PCDH10 | −3.078058405 | 1.09661E−07 | 0.002022473 | protocadherin 10 | NM_032961 |
| | | | | [Source: HGNC | |
| | | | | Symbol; Acc: 13404] | |
| PCDH7 | −1.647992519 | 2.26962E−08 | 0.000418586 | protocadherin 7 | NM_002589 |
| | | | | [Source: HGNC | |
| | | | | Symbol; Acc: 8659] | |
| PCSK5 | −1.835615027 | 7.72702E−07 | 0.014250945 | proprotein convertase | NM_006200 |
| | | | | subtilisin/kexin type 5 | |
| | | | | [Source: HGNC | |
| | | | | Symbol; Acc: 8747] | |
| PFKFB4 | −2.507828829 | 1.77486E−06 | 0.032733755 | 6-phosphofructo-2-kinase/fructose-2,6- | NM_004567 |
| | | | | biphosphatase 4 [Source: HGNC | |
| | | | | Symbol; Acc: 8875] | |
| PLA2G16 | 1.697662855 | 1.01793E−06 | 0.018773694 | phospholipase A2, group | NM_007069 |
| | | | | XVI [Source: HGNC | |
| | | | | Symbol; Acc: 17825] | |
| PLCD1 | 1.404173062 | 3.57371E−07 | 0.006590993 | phospholipase C, delta 1 | NM_001130964 |
| | | | | [Source: HGNC | |
| | | | | Symbol; Acc: 9060] | |
| PLCD3 | 0.873580218 | 9.70499E−08 | 0.001789891 | phospholipase C, delta 3 | NM_133373 |
| | | | | [Source: HGNC | |
| | | | | Symbol; Acc: 9061] | |
| PLEKHG1 | −1.049622185 | 1.55851E−07 | 0.002874369 | pleckstrin homology domain | NM_001029884 |
| | | | | containing, family G (with | |
| | | | | RhoGef domain) member 1 | |
| | | | | [Source: HGNC | |
| | | | | Symbol; Acc: 20884] | |
| PLK2 | −1.642092065 | 9.15471E−10 | 1.6884E−05 | polo-like kinase 2 | NM_006622 |
| | | | | [Source: HGNC | |
| | | | | Symbol; Acc: 19699] | |
| PMAIP1 | −2.96046458 | 2.46074E−10 | 4.53835E−06 | phorbol-12-myristate-13- | NM_021127 |
| | | | | acetate-induced | |
| | | | | protein 1 | |
| | | | | [Source: HGNC | |
| | | | | Symbol; Acc: 9108] | |
| PMEPA1 | −2.118128786 | 6.51157E−08 | 0.001200929 | prostate transmembrane | NM_020182 |
| | | | | protein, androgen induced 1 | |
| | | | | [Source: HGNC | |
| | | | | Symbol; Acc: 14107] | |
| PPARGC1A | 2.7347565 | 2.13568E−10 | 3.93883E−06 | peroxisome proliferator-activated | NM_013261 |
| | | | | receptor gamma, | |
| | | | | coactivator 1 alpha | |
| | | | | [Source: HGNC | |
| | | | | Symbol; Acc: 9237] | |
| PPP1R3G | 2.504955765 | 2.49358E−13 | 4.5989E−09 | protein phosphatase 1, | NM_001145115 |
| | | | | regulatory subunit 3G | |
| | | | | [Source: HGNC | |
| | | | | Symbol; Acc: 14945] | |
| PPP4R4 | −2.695005597 | 1.55476E−06 | 0.028674429 | protein phosphatase 4, | NM_058237 |
| | | | | regulatory subunit 4 | |
| | | | | [Source: HGNC | |
| | | | | Symbol; Acc: 23788] | |
| PRRG3 | −2.958900808 | 1.87199E−09 | 3.4525E−05 | proline rich Gla | NM_024082 |
| | | | | (G-carboxyglutamic acid) | |
| | | | | 3 (transmembrane) | |
| | | | | [Source: HGNC | |
| | | | | Symbol; Acc: 30798] | |
| PTHLH | −3.197986962 | 4.91519E−12 | 9.06509E−08 | parathyroid hormone-like | NM_198964 |
| | | | | hormone [Source: HGNC | |
| | | | | Symbol; Acc: 9607] | |
| PTX3 | −2.958231843 | 5.81537E−07 | 0.010725288 | pentraxin 3, long | NM_002852 |
| | | | | [Source: HGNC | |
| | | | | Symbol; Acc: 9692] | |
| RAB11A | 0.91182213 | 2.346E−06 | 0.043267306 | RAB11A, member RAS | NM_004663 |
| | | | | oncogene family | |
| | | | | [Source: HGNC | |
| | | | | Symbol; Acc: 9760] | |
| RAB3B | −2.490158066 | 5.83134E−07 | 0.010754749 | RAB3B, member RAS | NM_002867 |
| | | | | oncogene family | |
| | | | | [Source: HGNC | |
| | | | | Symbol; Acc: 9778] | |
| RASGEF1A | −2.347541016 | 9.39685E−09 | 0.000173306 | RasGEF domain family, | NM_001282862 |
| | | | | member 1A [Source: HGNC | |
| | | | | Symbol; Acc: 24246] | |
| RBFOX1 | 3.078820012 | 2.526E−06 | 0.046587089 | RNA binding protein, fox-1 | NM_018723 |
| | | | | homolog (C. elegans) 1 | |
| | | | | [Source: HGNC | |
| | | | | Symbol; Acc: 18222] | |
| RBMS3 | −1.363943847 | 2.44061E−06 | 0.045012249 | RNA binding motif, single | NM_001003793 |
| | | | | stranded interacting protein | |
| | | | | 3 [Source: HGNC | |
| | | | | Symbol; Acc: 13427] | |
| RIMS4 | −4.251537786 | 5.78005E−07 | 0.010660144 | regulating synaptic | NM_001205317 |
| | | | | membrane exocytosis 4 | |
| | | | | [Source: HGNC | |
| | | | | Symbol; Acc: 16183] | |
| RND1 | −4.788504478 | 5.89835E−08 | 0.001087832 | Rho family GTPase 1 | NM_014470 |
| | | | | [Source: HGNC | |
| | | | | Symbol; Acc: 18314] | |
| RNF144A | −1.050441709 | 2.32073E−06 | 0.042801267 | ring finger protein 144A | NM_014746 |
| | | | | [Source: HGNC | |
| | | | | Symbol; Acc: 20457] | |
| RNH1 | 0.597396408 | 5.27209E−07 | 0.009723323 | ribonuclease/angiogenin | NM_203383 |
| | | | | inhibitor 1 [Source: HGNC | |
| | | | | Symbol; Acc: 10074] | |
| RP11-166B2.7 | 4.324417845 | 2.21843E−06 | 0.040914449 | LNCipedia transcript ID: | Location (hg38): |
| | | | | lnc-NPIPB2-1:1 | chr16: 11976851-11977850 |
| RP11-195B3.1 | 4.629213869 | 5.4089E−10 | 9.97563E−06 | | |
| RP11-279F6.1 | 3.871662877 | 1.20831E−08 | 0.000222849 | | |
| RP11-359E10.1 | −2.649285371 | 1.56058E−06 | 0.028781769 | | |
| RP11-365H8.2 | 5.472328821 | 5.14932E−07 | 0.009496882 | | Entrez Gene: 145837 |
| | | | | | Ensembl: ENSG00000245750 |
| RP11-369C8.1 | 4.302568429 | 5.53269E−08 | 0.001020395 | | This transcript |
| | | | | | is a product of |
| | | | | | gene |
| | | | | | ENSG00000258616 |
| | | | | | HGNC: 53222 |
| RP11-379K22.3 | 4.814595052 | 1.80084E−11 | 3.32129E−07 | | Entrez Gene: 101929586 |
| RP11-395L14.4 | 2.380475471 | 3.46605E−07 | 0.006392436 | | Entrez Gene: 101927424 |
| | | | | | Ensembl:ENSG00000234148 |
| RP11-401P9.4 | −2.633011619 | 1.29967E−06 | 0.023969818 | | Ensembl ID: |
| | | | | | ENSG00000205414 |
| RP11-460N16.1 | 4.599284355 | 4.43741E−11 | 8.18392E−07 | | EnsEMBL Gene ID: |
| | | | | | ENSG00000240405 |
| RP11-474B16.1 | 4.848219082 | 1.84103E−08 | 0.000339542 | | |
| RP11-708H21.4 | 6.459588661 | 1.85913E−11 | 3.4288E−07 | | |
| RP11-737O24.5 | 1.842939217 | 5.48899E−09 | 0.000101234 | | |
| RP11-95P13.2 | 4.711600808 | 3.89648E−07 | 0.007186284 | | EnsEMBL Gene ID: |
| | | | | | ENSG00000238232 |
| RP3-438O4.4 | 4.739997657 | 1.40151E−07 | 0.002584798 | | |
| RPL17P22 | 4.158968001 | 6.01265E−09 | 0.000110891 | ribosomal protein L17 | HGNC: 35761 |
| | | | | pseudogene 22 | |
| RPS7 | 0.689333653 | 1.66546E−06 | 0.030716071 | ribosomal protein S7 | NM_001011 |
| | | | | [Source: HGNC | |
| | | | | Symbol; Acc: 10440] | |
| RTKN2 | −2.382849327 | 2.71874E−10 | 5.01417E−06 | rhotekin 2 [Source: HGNC | NM_145307 |
| | | | | Symbol; Acc: 19364] | |
| SAV1 | −0.562411769 | 1.69226E−06 | 0.031210396 | salvador homolog 1 | NM_021818 |
| | | | | (Drosophila) [Source: HGNC | |
| | | | | Symbol; Acc: 17795] | |
| SBK1 | −2.27009582 | 1.15377E−06 | 0.021278943 | SH3 domain binding kinase | NM_001024401 |
| | | | | 1 [Source: HGNC | |
| | | | | Symbol; Acc: 17699] | |
| SCD5 | −1.32594846 | 5.50495E−08 | 0.001015277 | stearoyl-CoA desaturase 5 | NM_001037582 |
| | | | | [Source: HGNC | |
| | | | | Symbol; Acc: 21088] | |
| SDS | −3.140396998 | 4.36991E−07 | 0.008059433 | serine dehydratase | NM_006843 |
| | | | | [Source: HGNC | |
| | | | | Symbol; Acc: 10691] | |
| SEC11C | 1.055944748 | 1.54531E−06 | 0.028500217 | SEC11 homolog C | NM_033280 |
| | | | | (S. cerevisiae) [Source: HGNC | |
| | | | | Symbol; Acc: 23400] | |
| SEMA3C | −1.8357644 | 1.57961E−06 | 0.029132704 | sema domain, | NM_006379 |
| | | | | immunoglobulin domain | |
| | | | | (Ig), short basic domain, | |
| | | | | secreted, (semaphorin) 3C | |
| | | | | [Source: HGNC | |
| | | | | Symbol; Acc: 10725] | |
| SEMA3D | −2.825717557 | 9.80981E−10 | 1.80922E−05 | sema domain, | NM_152754 |
| | | | | immunoglobulin domain | |
| | | | | (Ig), short basic domain, | |
| | | | | secreted, (semaphorin) 3D | |
| | | | | [Source: HGNC | |
| | | | | Symbol; Acc: 10726] | |
| SERPINA3 | −4.513242209 | 6.16231E−07 | 0.011365147 | serpin peptidase inhibitor, | NM_001085 |
| | | | | clade A (alpha-1 | |
| | | | | antiproteinase, antitrypsin), | |
| | | | | member 3 [Source: HGNC | |
| | | | | Symbol; Acc: 16] | |
| SERTM1 | −2.54678085 | 3.9235E−07 | 0.007236104 | serine-rich and | NM_203451 |
| | | | | transmembrane domain | |
| | | | | containing 1 [Source: HGNC | |
| | | | | Symbol; Acc: 33792] | |
| SHANK1 | −2.137808071 | 2.63258E−06 | 0.048552589 | SH3 and multiple ankyrin | NM_016148 |
| | | | | repeat domains 1 | |
| | | | | [Source: HGNC | |
| | | | | Symbol; Acc: 15474] | |
| SLC15A4 | 1.678198605 | 3.69524E−08 | 0.000681514 | solute carrier family 15 | NM_145648 |
| | | | | (oligopeptide transporter), | |
| | | | | member 4 [Source: HGNC | |
| | | | | Symbol; Acc: 23090] | |
| SLC16A9 | −1.475863342 | 1.23946E−06 | 0.022859273 | solute carrier family 16, | NM_194298 |
| | | | | member 9 [Source: HGNC | |
| | | | | Symbol; Acc: 23520] | |
| SLC1A1 | 3.929546374 | 4.31808E−12 | 7.96384E−08 | solute carrier family 1 | NM_004170 |
| | | | | (neuronal/epithelial high | |
| | | | | affinity glutamate | |
| | | | | transporter, system Xag), | |
| | | | | member 1 [Source: HGNC | |
| | | | | Symbol; Acc: 10939] | |
| SLC23A2 | −0.783173537 | 5.18646E−07 | 0.009565387 | solute carrier family 23 | NM_005116 |
| | | | | (ascorbic acid transporter), | |
| | | | | member 2 [Source: HGNC | |
| | | | | Symbol; Acc: 10973] | |
| SLC26A4 | −2.1787831 | 4.82827E−07 | 0.00890478 | solute carrier family 26 | NM_000441 |
| | | | | (anion exchanger), member | |
| | | | | 4 [Source: HGNC | |
| | | | | Symbol; Acc: 8818] | |
| SLC44A5 | −2.16133252 | 2.17951E−07 | 0.004019671 | solute carrier family 44, | NM_001130058 |
| | | | | member 5 [Source: HGNC | |
| | | | | Symbol; Acc: 28524] | |
| SLC47A1 | −4.221015034 | 1.28272E−12 | 2.36571E−08 | solute carrier family 47 | NM_018242 |
| | | | | (multidrug and toxin | |
| | | | | extrusion), member 1 | |
| | | | | [Source: HGNC | |
| | | | | Symbol; Acc: 25588] | |
| SLC52A3 | −1.6116801 | 1.94516E−06 | 0.035874555 | solute carrier family 52 | NM_033409 |
| | | | | (riboflavin transporter), | |
| | | | | member 3 [Source: HGNC | |
| | | | | Symbol; Acc: 16187] | |
| SMAD7 | −1.125990865 | 6.71143E−08 | 0.001237789 | SMAD family member 7 | NM_001190821 |
| | | | | [Source: HGNC | |
| | | | | Symbol; Acc: 6773] | |
| SNX29 | 1.324410276 | 7.77371E−07 | 0.014337048 | sorting nexin 29 | NM_032167 |
| | | | | [Source: HGNC | |
| | | | | Symbol; Acc: 30542] | |
| SPRY1 | −1.381394885 | 1.83739E−11 | 3.3887E−07 | sprouty homolog 1, | NM_199327 |
| | | | | antagonist of FGF signaling | |
| | | | | (Drosophila) [Source: HGNC | |
| | | | | Symbol; Acc: 11269] | |
| SPTLC3 | 1.764525816 | 5.97397E−09 | 0.000110178 | serine palmitoyltransferase, | NM_018327 |
| | | | | long chain base subunit 3 | |
| | | | | [Source: HGNC | |
| | | | | Symbol; Acc: 16253] | |
| SPTSSA | 1.063193649 | 2.20317E−06 | 0.040633127 | serine palmitoyltransferase, | NM_138288 |
| | | | | small subunit A | |
| | | | | [Source: HGNC | |
| | | | | Symbol; Acc: 20361] | |
| SSTR2 | −2.637606703 | 1.41042E−06 | 0.026012342 | somatostatin receptor 2 | NM_001050 |
| | | | | [Source: HGNC | |
| | | | | Symbol; Acc: 11331] | |
| STAC2 | 2.943564818 | 1.69006E−06 | 0.031169741 | SH3 and cysteine rich | NM_198993 |
| | | | | domain 2 [Source: HGNC | |
| | | | | Symbol; Acc: 23990] | |
| TACSTD2 | −2.386152793 | 5.29224E−10 | 9.76047E−06 | tumor-associated calcium | NM_002353 |
| | | | | signal transducer 2 | |
| | | | | [Source: HGNC | |
| | | | | Symbol; Acc: 11530] | |
| TBCK | 0.989806512 | 7.39948E−07 | 0.01364687 | TBC1 domain containing | NM_033115 |
| | | | | kinase [Source: HGNC | |
| | | | | Symbol; Acc: 28261] | |
| TCF7 | −1.399170558 | 4.86475E−09 | 8.97206E−05 | transcription factor 7 (T-cell | NM_003202 |
| | | | | specific, HMG-box) | |
| | | | | [Source: HGNC | |
| | | | | Symbol; Acc: 11639] | |
| TEX101 | 5.499336183 | 3.56223E−07 | 0.006569817 | testis expressed 101 | NM_031451 |
| | | | | [Source: HGNC | |
| | | | | Symbol; Acc: 30722] | |
| TIFA | −1.0276487 | 7.70928E−07 | 0.014218234 | TRAF-interacting protein | NM_052864 |
| | | | | with forkhead-associated | |
| | | | | domain [Source: HGNC | |
| | | | | Symbol; Acc: 19075] | |
| TMEM120B | −1.305469417 | 3.61619E−07 | 0.006669343 | transmembrane protein | NM_001080825 |
| | | | | 120B [Source: HGNC | |
| | | | | Symbol; Acc: 32008] | |
| TMEM215 | −4.696018074 | 7.15366E−08 | 0.00131935 | transmembrane protein 215 | NM_212558 |
| | | | | [Source: HGNC | |
| | | | | Symbol; Acc: 33816] | |
| TMEM63C | 2.707880925 | 8.23759E−07 | 0.01519258 | transmembrane protein 63C | NM_020431 |
| | | | | [Source: HGNC | |
| | | | | Symbol; Acc: 23787] | |
| TNFAIP3 | −2.497918892 | 2.67567−07 | 0.004934731 | tumor necrosis factor, | NM_001270507 |
| | | | | alpha-induced protein 3 | |
| | | | | [Source: HGNC | |
| | | | | Symbol; Acc: 11896] | |
| TNFRSF12A | −1.949619966 | 2.25476E−06 | 0.041584474 | tumor necrosis factor | NM_016639 |
| | | | | receptor superfamily, | |
| | | | | member 12A | |
| | | | | [Source: HGNC | |
| | | | | Symbol; Acc: 18152] | |
| TNFSF9 | −2.572591499 | 5.31221E−07 | 0.009797313 | tumor necrosis factor | NM_003811 |
| | | | | (ligand) superfamily, | |
| | | | | member 9 [Source: HGNC | |
| | | | | Symbol; Acc: 11939] | |
| TNS3 | −1.038470044 | 4.65742E−09 | 8.58968E−05 | tensin 3 [Source: HGNC | NM_022748 |
| | | | | Symbol; Acc: 21616] | |
| TPSAB1 | −4.118567619 | 2.19378E−07 | 0.004045986 | tryptase alpha/beta 1 | NM_003294 |
| | | | | [Source: HGNC | |
| | | | | Symbol; Acc: 12019] | |
| TPSB2 | −3.805796899 | 9.01075E−07 | 0.016618531 | tryptase beta 2 | NM_024164 |
| | | | | (gene/pseudogene) | |
| | | | | [Source: HGNC | |
| | | | | Symbol; Acc: 14120] | |
| TRIOBP | 0.896036545 | 3.19451E−11 | 5.89164E−07 | TRIO and F-actin binding | NM_001039141 |
| | | | | protein [Source: HGNC | |
| | | | | Symbol; Acc: 17009] | |
| TSPAN8 | 3.539833205 | 4.53393E−08 | 0.000836193 | tetraspanin 8 [Source: HGNC | NM_004616 |
| | | | | Symbol; Acc: 11855] | |
| UCN2 | −2.830072659 | 1.61225E−06 | 0.029734726 | urocortin 2 [Source: HGNC | NM_033199 |
| | | | | Symbol; Acc: 18414] | |
| UGT1A6 | 3.849213299 | 1.01728E−06 | 0.018761613 | UDP | NM_205862 |
| | | | | glucuronosyltransferase 1 | |
| | | | | family, polypeptide A6 | |
| | | | | [Source: HGNC | |
| | | | | Symbol; Acc: 12538] | |
| USP6NL | −0.717252648 | 1.72797E−06 | 0.031868903 | USP6 N-terminal like | NM_014688 |
| | | | | [Source: HGNC | |
| | | | | Symbol; Acc: 16858] | |
| VANGL2 | −1.678938079 | 5.20305E−10 | 9.59598E−06 | VANGL planar cell polarity | NM_020335 |
| | | | | protein 2 [Source: HGNC | |
| | | | | Symbol; Acc: 15511] | |
| VASH2 | −2.170543027 | 2.73609E−07 | 0.005046163 | vasohibin 2 [Source: HGNC | NM_001136474 |
| | | | | Symbol; Acc: 25723] | |
| VTN | 1.940794256 | 7.75529E−07 | 0.014303087 | vitronectin [Source: HGNC | NM_000638 |
| | | | | Symbol; Acc: 12724] | |
| VWA2 | −1.670171542 | 2.97931E−09 | 5.49474E−05 | von Willebrand factor A | NM_001272046 |
| | | | | domain containing 2 | |
| | | | | [Source: HGNC | |
| | | | | Symbol; Acc: 24709] | |
| WISP1 | −3.698212804 | 2.18136E−11 | 4.02308E−07 | WNT1 inducible signaling | NM_003882 |
| | | | | pathway protein 1 | |
| | | | | [Source: HGNC | |
| | | | | Symbol; Acc: 12769] | |
| WNT5A-AS1 | −2.364563723 | 1.25625E−07 | 0.00231691 | WNT5A antisense RNA 1 | Not shown |
| | | | | [Source: HGNC | |
| | | | | Symbol; Acc: 40616] | |
| ZBED6CL | −1.832151237 | 7.23703E−09 | 0.000133473 | ZBED6 C-terminal like | NM_138434 |
| | | | | [Source: HGNC | |
| | | | | Symbol; Acc: 21720] | |
| ZCCHC14 | −0.869606159 | 1.27614E−08 | 0.000235359 | zinc finger, CCHC domain | NM_015144 |
| | | | | containing 14 | |
| | | | | [Source: HGNC | |
| | | | | Symbol; Acc: 24134] | |
| ZMYND15 | −2.123911647 | 5.81655E−07 | 0.010727463 | zinc finger, MYND-type | NM_001136046 |
| | | | | containing 15 | |
| | | | | [Source: HGNC | |
| | | | | Symbol; Acc: 20997] | |
| ZNF469 | −2.187278102 | 2.44962E−06 | 0.045178354 | zinc finger protein 469 | NM_001127464 |
| | | | | [Source: HGNC | |
| | | | | Symbol; Acc: 23216] | |
| ZNF608 | −1.280523081 | 4.50326E−09 | 8.30537E−05 | zinc finger protein 608 | NM_020747 |
| | | | | [Source: HGNC | |
| | | | | Symbol; Acc: 29238] | |
| ZNF827 | −0.968850227 | 2.47586E−06 | 0.045662327 | zinc finger protein 827 | NM_178835 |
| | | | | [Source: HGNC | |
| | | | | Symbol; Acc: 27193] | |
| ZPLD1 | 4.259693827 | 2.14156E−08 | 0.000394967 | zona pellucida-like domain | NM_175056 |
| | | | | containing 1 [Source: HGNC | |
| | | | | Symbol; Acc: 27022] | |

In another embodiment, the biomarkers are one or more (e.g., all or substantially all) of those defined in Table C. In some embodiments, the biomarkers represent a set of 15 differentially expressed genes (“DEGs”) from biological samples taken from patients with prior severe pre-eclampsia (sPE) compared to control biological tissues taken from term patients not having sPE. In various embodiments, the biological samples are endometrial samples, which may comprise endometrial tissue, endometrial cells, and/or endometrial fluids. In still other embodiments, the biological sample can be blood. Table C biomarkers include:

TABLE C
Global RNAseq: sPE versus Control Term

| | | logFC | | | | | |
|---|---|---|---|---|---|---|---|
| | | (log 2 | | | | RefSeq | |
| | HGNC | of Fold | | P-Value | Gene | mRNA | RefSeq |
| Gene name | Symbol | Change) | P-Value | adjusted | description | ID | peptide ID |
| CTTNBP2 | CTTNBP2 | −1.12 | 7.8033E−07 | 0.014165399 | Cortactin | NM_033427 | NP_219499.1 |
| | | | | | Binding | | |
| | | | | | Protein 2 | | |
| TACSTD2 | TACSTD2 | −1.91 | 6.5402E−08 | 0.001187251 | tumor- | NM_002353 | NP_002344 |
| | | | | | associated | | |
| | | | | | calcium signal | | |
| | | | | | transducer 2 | | |
| | | | | | [Source:HGNC | | |
| | | | | | Symbol; | | |
| | | | | | Acc:11530] | | |
| ZMYND15 | ZMYND15 | −2.19 | 6.7544E−07 | 0.012261345 | zinc finger, | NM_001136046 | NP_001254751 |
| | | | | | MYND-type | | |
| | | | | | containing 15 | | |
| | | | | | [Source:HGNC | | |
| | | | | | Symbol; | | |
| | | | | | Acc:20997] | | |
| AC116366.6 | AC116366.6 | −2.43 | 2.6003E−06 | 0.047204053 | Transcript: | ENST00000443093.2 | NO |
| | | | | | AC116366.1- | | PROTEIN |
| | | | | | 201 | | |
| RRAD | RRAD | −2.68 | 1.9653E−06 | 0.035676969 | Ras-related | NM_001128850 | NP_004156 |
| | | | | | associated | | |
| | | | | | with diabetes | | |
| | | | | | [Source:HGNC | | |
| | | | | | Symbol; | | |
| | | | | | Acc:10446] | | |
| LCN2 | LCN2 | −2.76 | 6.1622E−10 | 1.11863E−05 | lipocalin 2 | NM_005564 | NP_005555 |
| | | | | | [Source:HGNC | | |
| | | | | | Symbol; | | |
| | | | | | Acc:6526] | | |
| CXCL1 | CXCL1 | −2.92 | 1.1628E−06 | 0.021108675 | chemokine | NM_001511 | NP_001502 |
| | | | | | (C—X—C motif) | | |
| | | | | | ligand 1 | | |
| | | | | | (melanoma | | |
| | | | | | growth | | |
| | | | | | stimulating | | |
| | | | | | activity, alpha) | | |
| | | | | | [Source:HGNC | | |
| | | | | | Symbol; | | |
| | | | | | Acc:4602] | | |
| RND1 | RND1 | −3.29 | 5.8486E−08 | 0.0010617 | Rho family | NM_014470 | NP_055285 |
| | | | | | GTPase 1 | | |
| | | | | | [Source:HGNC | | |
| | | | | | Symbol; | | |
| | | | | | Acc:18314] | | |
| CCL20 | CCL20 | −3.32 | 1.3729E−06 | 0.024921764 | chemokine | NM_001130046 | NP_004582 |
| | | | | | (C-C motif) | | |
| | | | | | ligand 20 | | |
| | | | | | [Source:HGNC | | |
| | | | | | Symbol; | | |
| | | | | | Acc:10619] | | |
| IL6 | IL6 | −3.65 | 7.2578E−09 | 0.000131751 | interleukin 6 | NM_000600 | NP_000591 |
| | | | | | (interferon, | | |
| | | | | | beta 2) | | |
| | | | | | [Source:HGNC | | |
| | | | | | Symbol; | | |
| | | | | | Acc:6018] | | |
| LTF | LTF | −3.73 | 1.6012E−07 | 0.002906628 | lactotransferrin | NM_002343 | NP_002334 |
| | | | | | [Source:HGNC | | |
| | | | | | Symbol; | | |
| | | | | | Acc:6720] | | |
| SAA1 | SAA1 | −4.01 | 1.2383E−10 | 2.24796E−06 | serum amyloid | NM_199161 | NP_954630 |
| | | | | | A1 | | |
| | | | | | [Source:HGNC | | |
| | | | | | Symbol; | | |
| | | | | | Acc:10513] | | |
| hsa-mir- | MIR6723 | −4.18 | 3.313E−08 | 0.000601411 | MicroRNA | ENSG00000278791 | |
| 6723 | | | | | 6723 | | |
| ADRA2B | ADRA2B | −4.49 | 3.7757E−07 | 0.00685401 | adrenoceptor | NM_000682 | NP_000673 |
| | | | | | alpha 2B | | |
| | | | | | [Source:HGNC | | |
| | | | | | Symbol; | | |
| | | | | | Acc:282] | | |
| MTND1P23 | MTND1P23 | −5.35 | 8.5823E−08 | 0.001557938 | Homo sapiens | NG_032769.1 | |
| | | | | | MT-ND1 | | |
| | | | | | pseudogene 23 | | |
| | | | | | (MTND1P23) on | | |
| | | | | | chromosome 1. | | |

The biomarkers described herein may have a level in a sample obtained from a subject (e.g., patient) that had preeclampsia in a previous pregnancy that deviates (e.g., is increased or reduced) when compared to the level of the same biomarker in a sample obtained from a woman that had a normal pregnancy by at least 20% (e.g., 30%, 50%, 80%, 100%, 2-fold, 5-fold, 10-fold, 20-fold, 50-fold, 100-fold or more). The biomarkers described herein may have a level in decidualized cells that deviates (e.g., is increased or reduced) from the level of the same marker in non-decidualized cells by at least 20% (e.g., 30%, 50%, 80%, 100%, 2-fold, 5-fold, 10-fold, 20-fold, 50-fold, 100-fold or more). Such a biomarker or set of biomarkers may be used in both diagnostic/prognostic applications and non-clinical applications (e.g., for research purposes).

In some embodiments, methods described herein provide determining a level of between 1 to 129 biomarkers indicative of preeclampsia from Table 1, and/or between 1 to 36 biomarkers indicative of preeclampsia from Table A, and/or between 1-246 biomarkers indicative of preeclampsia from Table B, and/or between 1-15 biomarkers indicative of preeclampsia from Table C. For example, in some embodiments, methods described herein provide determining a level of between 5 to 129 biomarkers, between 10 to 129 biomarkers, between 15 to 129 biomarkers, between 25 to 129 biomarkers, between 50 to 129 biomarkers, between 75 to 129 biomarkers, between 100 to 129 biomarkers, or between 125 to 129 biomarkers indicative of preeclampsia from Table 1. For example, in some embodiments, methods described herein provide determining a level of between 5 to 36 biomarkers, between 10 to 36 biomarkers, between 15 to 36 biomarkers, between 25 to 36 biomarkers, between 10-15 biomarkers, between 15 to 25 biomarkers, between 25 to 30 biomarkers, or between 30-36 biomarkers indicative of preeclampsia from Table A. In some embodiments, methods described herein provide determining a level of between 5 to 246 biomarkers, between 10 to 246 biomarkers, between 15 to 246 biomarkers, between 25 to 246 biomarkers, between 50 to 246 biomarkers, between 75 to 246 biomarkers, between 100 to 246 biomarkers, between 125 to 246 biomarkers, between 150 to 246 biomarkers, or between 200 to 246 biomarkers indicative of preeclampsia from Table B. In some embodiments, methods described herein provide determining a level of between 5 to 15 biomarkers, between 10 to 15 biomarkers, or between 5 to 10 biomarkers indicative of preeclampsia from Table C. In some embodiments, a combination of biomarkers from different tables are used. In some embodiments, methods described herein provide determining a level of between 1 to 125 biomarkers, between 1 to 100 biomarkers, between 1 to 75 biomarkers, between 1 to 50 biomarkers, between 1 to 25 biomarkers, between 1 to 15 biomarkers, between 1 to 10 biomarkers, or between 1 to 5 biomarkers indicative of preeclampsia (e.g., from one or more of Tables 1, A, B, and/or C).

In some embodiments, methods described herein provide determining a level of at least one biomarker, at least 2 biomarkers, at least 3 biomarkers, at least 4 biomarkers, at least 5 biomarkers, at least 6 biomarkers, at least 7 biomarkers, at least 8 biomarkers, at least 9 biomarkers, or at least 10 biomarkers indicative of preeclampsia.

In some embodiments, methods described herein provide determining a level of less than 500 biomarkers, less than 450 biomarkers, less than 400 biomarkers, less than 350 biomarkers, less than 300 biomarkers, less than 250 biomarkers, less than 200 biomarkers, less than 150 biomarkers, less than 100 biomarkers, less than 50 biomarkers, less than 25 biomarkers, or less than 5 biomarkers.

In various embodiments, the biomarkers that may be used herein include any combination of biomarkers from Tables 1, A, B, and C. For example, the methods described herein may utilize one or more biomarkers from Table 1 in combination with one or more biomarkers from Table A. In another example, the methods described herein may utilize one or more biomarkers from Table 1 in combination with one or more biomarkers from Table B. In still another example, the methods described herein may utilize one or more biomarkers from Table 1 in combination with one or more biomarkers from Table C. In still another example, the methods described herein may use one or more biomarkers from Table 1 in combination with one or more biomarkers from Table A, and/or one or more biomarkers from Table B, and/or one more biomarkers from Table C. The methods may use any combination of biomarkers from Tables 1, A, B, and C, in combination with any other biomarkers disclosed herein not included in Tables 1, A, B, or C.

In some embodiments, methods described herein provide determining a level of at least one biomarker selected from a group of biomarkers indicative of preeclampsia. In some embodiments, methods described herein provide determining a level of at least one biomarker selected from two or more groups of biomarkers indicative of preeclampsia. Groups of biomarkers may consist essentially of at least 3 biomarkers, at least 5 biomarkers, at least 9 biomarkers, at least 22 biomarkers, at least 50 biomarkers, or at least 100 biomarkers indicative of preeclampsia.

The methods and compositions described herein may comprise, consist of, or consist essentially of any combination or number of the described biomarkers or biomarker groups, without limitation. As used herein, a group of biomarkers “consisting essentially of” a list of specified genes or gene products will include the genes (e.g., biomarkers) recited in the group, and may include one or more inconsequential or control genes (e.g., biomarkers) that do not materially affect the basic and novel characteristics of the claimed group. In some embodiments, one or more control genes (e.g., biomarkers) may be, for example, one or more housekeeping genes. In some embodiments, the control gene (e.g., biomarker) is a positive control. In some embodiments, the control gene (e.g., biomarker) is a negative control. In some embodiments, one or more control gene (e.g., biomarker) comprises a detection control. In some embodiments, the detection control is a labeled nucleic acid. In some embodiments, the detection control is a labeled antibody. In some embodiments, the detection control is a protein with a detectable label.

Biomarkers may be grouped based on one or more characteristics of a particular biomarker. In some embodiments, biomarkers are grouped based on expression of the biomarker in a particular patient population (e.g., women that had a pregnancy complicated with preeclampsia). In some embodiments, biomarkers are grouped based on expression of the biomarker in a particular cell (e.g., human endometrial stromal cells (hESCs)). In some embodiments, biomarkers are grouped based on expression of the biomarker in a particular tissue (e.g., decidua basalis or decidua parietalis). In some embodiments, biomarkers are grouped based on expression of the biomarker during a particular cellular process (e.g., decidualization). In some embodiments, biomarkers are grouped based on an association with a particular pathway (e.g., extracellular structure organization). In some embodiments, biomarkers are grouped based on a known function of the biomarker. In some embodiments, biomarkers are grouped based on absolute value of the ratio of a determined level of the biomarker to a control level of the biomarker.

In some embodiments, a group of biomarkers comprises, consists of, or consists essentially of CNR1, IRS2, CHST7, TSC22D3, PRUNE2, ADAMTS8, MAOA, MGST1, FKBP5, SCARA5, ZBTB16, GLUL, SERPINA3, NPR1, LPAR1, APOD, ABLIM2, CHI3L2, PDLIM1, PID1, TIMP4, ACSL1, LTBP1, TNFRSF8, SLC27A3, ABCB4, GPC2, SBK1, TRO, TSPAN6, DOCK6, GNB1L, SOX4, ZSWIM4, PODXL, SERTAD4, LMO2, FOXL2, AFAP1L2, COCH, GPRC5C, FBXO2, Clorf133, TMSB15A, GFRA2, PRAGMIN, TSPAN11, CNIH3, F2RL1, and DI02, optionally in combination with one or more additional biomarkers from any of Tables 1, A, B, or C.

In some embodiments, a group of biomarkers comprises, consists of, or consists essentially of HSD17B2, ANGPT2, NCKAP5, ADRA2A, DBC1, C1QTNF7, COL8A1, EGR1, SSTR1, FBXO2, CPE, C4orf49, GRP, IGFBP5, COCH, ARHGDIB, SCG5, ITGA11, SLC35F3, RLN2, COL14A1, CLIC3, TMEM25, CCDC81, MYCN, NPR1, RASGRP2, CHI3L2, RSPO3, C1Oorf10, TMEM132C, PPAP2B, NKAIN1, ADAMTS8, IL15, SLC7A2, SERPINA3, NPTX1, CHST7, GALNTL2, SBSN, EDNRA, IL1B, SPARCL1, SCARA5, SIPA1L2, CCL8, P2RY14, CNR1, and IGFBP1, optionally in combination with one or more additional biomarkers from any of Tables 1, A, B, or C.

In some embodiments, a group of biomarkers comprises, consists of, or consists essentially of LOC101928439, RP11-1026M7.3, RNU4ATAC18P, TRBV4-2, RP11-12D16.2, TRAJ59, RNU4-39P, RNU6-540P, RNA5SP187, PRKXP1, MIR4509-1, RNU6-1111P, A1BG-AS1, CSPG4, MIR365A, RNA5SP463, BACE1-AS, RNU6-621P, RNU4-76P, TRIM48, PSMD3, RP11-661Al2.4, LOC644172, ZNF483, ARL5B, ENPP4, IPW, SPINK1, C7, SNORD52, CYP19A1, TSPAN1, LOC101929607, SNORD52, RNU2-5P, MS4A2, SNORD71, RNU6V, RNU6-901P, MME-AS1, TAS2R46, MIR548H1, COL8A1, SNORD115-32, UGT2B7, OGN, RP11-872D17.8, RP11-108K3.1, CP, and DEFB1, optionally in combination with one or more additional biomarkers from any of Tables 1, A, B, or C.

In some embodiments, a group of biomarkers comprises, consists of, or consists essentially of PRG2, AC073218.2, AC073218.3, RNASE2, LOC100506530, A0X1, PZP, RP11-57P19.1, LINCO1338, NOTUM, TMEM27, CTC-498J12.1, IGSF10, KLRF1, TRPC4, GPR126, ADAMTS15, PROM1, PDGFD, KIR2DL2, LOC101929174, SULF2, MUM1L1, ACE2, SAPCD1, RP11-59H7.3, DOCK4-AS1, GBP2, TNC, XXbac-BPG252P9.10, RNU6-1024P, MT1CP, RN7SKP16, IER3, INHBA, DSC3, SERPINB11, RP1-68D18.4, ILIA, BMP2, ADAMTS4, LINC00312, MMP10, RNU6-162P, CXCL5, ICAM1, RNU7-40P, SPINK1, IL23A, and CXCL8, optionally in combination with one or more additional biomarkers from any of Tables 1, A, B, or C.

In some embodiments, a group of biomarkers comprises, consists of, or consists essentially of HSD17B2, ANGPT2, NCKAP5, ADRA2A, DBC1, C1QTNF7, COL8A1, EGR1, SSTR1, FBXO2, CPE, C4orf49, GRP, IGFBP5, COCH, ARHGDIB, SCG5, ITGA11, SLC35F3, RLN2, COL14A1, CLIC3, TMEM25, CCDC81, MYCN, SLITRK6, TTR, ISM1, PITX1, SULF1, OXTR, AADAC, MEST, C17orf107, CNIH3, HMCN1, Clorf133, MYLK, CLEC3B, F2RL2, ADAMTS19, ATCAY, BDNF, DUSP6, KLF2, REEP2, DENND2A, LPL, KRTAP17-1, LOXL4, NANOS3, OLFML1, C14orf37, ENST00000313664, LAMAS, LYPD1, GBP2, FAM19A2, SERTAD4, CHODL, ERAP2, ERP27, FAM38B, GALNT14, LOC728392, PDGFD, FAT1, TNFRSF10C, EHD3, MFAP2, MRVI1, TNFAIP6, FST, DMKN, ANXA2, DES, EFEMP1, RGS20, CA12, GGT5, ENST00000380464, LTBP1, C6orf176, TNFRSF8, BAIAP2L2, LSAMP, DDIT4, RHOU, IRS2, EDNRB, COL15A1, DCN, WNT6, LPAR1, RGS16, KCNJ8, ABLIM2, LRRC15, CRLF1, RASL11B, CFD, GAL, ALDH1A1, PRUNE2, NPR1, RASGRP2, CHI3L2, RSPO3, C1Oorf10, TMEM132C, PPAP2B, NKAIN1, ADAMTS8, IL15, SLC7A2, SERPINA3, NPTX1, CHST7, GALNTL2, SBSN, EDNRA, IL1B, SPARCL1, SCARA5, SIPA1L2, CCL8, P2RY14, CNR1, and IGFBP1, optionally in combination with one or more additional biomarkers from any of Tables 1, A, B, or C.

In some embodiments, a group of biomarkers comprises, consists of, or consists essentially of CNR1, IRS2, CHST7, PRUNE2, ADAMTS8, SCARA5, SERPINA3, NPR1, LPAR1, ABLIM2, CHI3L2, LTBP1, TNFRSF8, SLC27A3, ILI, CCDC, PPAP2C, SERTADA4, COCH, FBXO2, Clorf133, and CNIH3, optionally in combination with one or more additional biomarkers from any of Tables 1, A, B, or C.

In some embodiments, a group of biomarkers comprises, consists of, or consists essentially of ADAMTS8, CHI3L2, CHST7, CNR1, COCH, FBXO2, NPR1, SCARA5, and SERPINA3, optionally in combination with one or more additional biomarkers from any of Tables 1, A, B, or C.

Any number and/or combination of the biomarkers listed herein or the groups of biomarkers listed herein may be used in the described methods and/or devices. For example, the group of biomarkers used in the method or assay may comprise, consist of, or essentially consist of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, or more of the biomarkers listed herein.

In some embodiments the group of biomarkers used in the method or assay may comprise, consist of, or essentially consist of more than 10, more than 11, more than 12, more than 13, more than 14, more than 15, more than 16, more than 17, more than 18, more than 19, more than 20, more than 21, more than 22, more than 23, more than 24, more than 25, more than 26, more than 27, more than 28, more than 29, more than 30, more than 31, more than 32, more than 33, more than 34, more than 35, more than 36, more than 37, more than 38, more than 39, more than 40, more than 41, more than 42, more than 43, more than 44, more than 45, more than 46, more than 47, more than 48, more than 49, more than 50, more than 51, more than 52, more than 53, more than 54, more than 55, more than 56, more than 57, more than 58, more than 59, more than 60, more than 61, more than 62, more than 63, more than 64, more than 65, more than 66, more than 67, more than 68, more than 69, more than 70, more than 71, more than 72, more than 73, more than 74, more than 75, more than 76, more than 77, more than 78, more than 79, more than 80, more than 81, more than 82, more than 83, more than 84, more than 85, more than 86, more than 87, more than 88, more than 89, more than 90, more than 91, more than 92, more than 93, more than 94, more than 95, more than 96, more than 97, more than 98, more than 99, more than 100, more than 101, more than 102, more than 103, more than 104, more than 105, more than 106, more than 107, more than 108, more than 109, more than 110, more than 111, more than 112, more than 113, more than 114, more than 115, more than 116, more than 117, more than 118, more than 119, more than 120, more than 121, more than 122, more than 123, more than 124, more than 125, more than 126, more than 127, more than 128, or more than 129 of the biomarkers listed herein.

In some embodiments the group of biomarkers used in the method or assay may comprise, consist of, or essentially consist of no more than 10, no more than 11, no more than 12, no more than 13, no more than 14, no more than 15, no more than 16, no more than 17, no more than 18, no more than 19, no more than 20, no more than 21, no more than 22, no more than 23, no more than 24, no more than 25, no more than 26, no more than 27, no more than 28, no more than 29, no more than 30, no more than 31, no more than 32, no more than 33, no more than 34, no more than 35, no more than 36, no more than 37, no more than 38, no more than 39, no more than 40, no more than 41, no more than 42, no more than 43, no more than 44, no more than 45, no more than 46, no more than 47, no more than 48, no more than 49, no more than 50, no more than 51, no more than 52, no more than 53, no more than 54, no more than 55, no more than 56, no more than 57, no more than 58, no more than 59, no more than 60, no more than 61, no more than 62, no more than 63, no more than 64, no more than 65, no more than 66, no more than 67, no more than 68, no more than 69, no more than 70, no more than 71, no more than 72, no more than 73, no more than 74, no more than 75, no more than 76, no more than 77, no more than 78, no more than 79, no more than 80, no more than 81, no more than 82, no more than 83, no more than 84, no more than 85, no more than 86, no more than 87, no more than 88, no more than 89, no more than 90, no more than 91, no more than 92, no more than 93, no more than 94, no more than 95, no more than 96, no more than 97, no more than 98, no more than 99, no more than 100, no more than 101, no more than 102, no more than 103, no more than 104, no more than 105, no more than 106, no more than 107, no more than 108, no more than 109, no more than 110, no more than 111, no more than 112, no more than 113, no more than 114, no more than 115, no more than 116, no more than 117, no more than 118, no more than 119, no more than 120, no more than 121, no more than 122, no more than 123, no more than 124, no more than 125, no more than 126, no more than 127, no more than 128, or no more than 129 of the biomarkers listed herein.

In some embodiments, a group of biomarkers is associated with at least one of the following pathways: extracellular structure organization, tissue development, inflammation, immune function, transport and/or metabolism, cell signaling, transcription and/or translation, signal transduction, protein degradation, insulin related, G-protein signaling, and cell cycle and activation.

In some embodiments, a group of biomarkers associated with an extracellular structure organization pathway comprises, consists of, or consists essentially of LAMAS, DMKN, CCDC81, DES, LAMAS, SULF1, ITGA11, COL8A1, COL14A1, MFAP2, BAIAP2L2, MRVI1, TMEM132C, and TMEM25.

In some embodiments, a group of biomarkers associated with a tissue development pathway comprises, consists of, or consists essentially of SLITRK6, CHODL, MEST, and SULF1.

In some embodiments, a group of biomarkers associated with an inflammation pathway comprises, consists of, or consists essentially of CXCL8, IL23A, ILIA, CXCLS, and CCL8.

In some embodiments, a group of biomarkers associated with an immune function pathway comprises, consists of, or consists essentially of TNFRSF10C, TNFRSF8, ADRA2A, COCH, FAN19A2, GAL, GBP2, IL1B, IL15, LSAMP, SERPINA, and SLC7A2.

In some embodiments, a group of biomarkers associated with a transport and/or metabolism pathway comprises, consists of, or consists essentially of ALDH1A1, AADAC, CNR1, CHST7, CA12, CPE, CHI3L2, CLIC3, HSD17B2, LPL, NPR1, GALNT14, PRUNE2, OXTR, TTR, ATCAY, DENND2A, NKAIN1, CNIH3, NPTX1, KCNJ8, REEP2, SCG5, SLC35F3, and ERAP2.

In some embodiments, a group of biomarkers associated with a cell signaling pathway comprises, consists of, or consists essentially of LTBP1, F2RL2, FAT1, ANXA2, BDNF, DCN, EDNRA, EDNRB, ITGA11, LRRC15, RKB2, SSTR1 RSPO3, WNT6, LPAR1, PDGFD, RHOU, MYLK, DDIT4, ARHGDIB, and DUSP6.

In some embodiments, a group of biomarkers associated with a transcription and translation pathway comprises, consists of, or consists essentially of EFEMP1, KLF2, ABLIM2, EGR1, FST, PITX1, NTCB, and NANOS3.

In some embodiments, a group of biomarkers associated with a signal transduction pathway comprises, consists of, or consists essentially of SPARCL1, TNFAIP6, ANGPT2, COL15A1, and GRP.

In some embodiments, a group of biomarkers associated with a protein degradation pathway comprises, consists of, or consists essentially of ERP27, ADAMTS19, ADAMTS8, FBXO2, CFD, GGT5, EHD3, LOXL4, and SCARA5.

In some embodiments, a group of biomarkers associated with an insulin related pathway comprises, consists of, or consists essentially of IGFBP1, IGFBP5, and IRS2.

In some embodiments, a group of biomarkers associated with a G-protein signaling pathway comprises, consists of, or consists essentially of P2RY14, RGS20, RASGRP2, RASL11B, RGS16, and SIPA1L2.

In some embodiments, a group of biomarkers associated with a cell cycle and activation pathway comprises, consists of, or consists essentially of LYPD1, HMCN1, CRLF1, and CLE3B.

In some embodiments, a group of biomarkers associated with an unspecified pathway comprises, consists of, or consists essentially of C1QTNF7, NCKAP5, SERTAD4, C1Oorf10, C14orf37, Cl7orf107, ISM1, OLFML1, and SBSN.

In some embodiments, a group of biomarkers having an absolute value of the ratio of a determined level of the biomarker to a control level of the biomarker greater than 10 comprises, consists of, or consists essentially of CNR1, IRS2, CHST7, TSC22D3, PRUNE2, HSD17B2, ANGPT2, NCKAP5, ADRA2A, DBC1, C1QTNF7, SPARCL1, SCARA5, SIPA1L2, CCL8, P2RY14, CNR1, IGFBP1, CP, DEFB1, PRG2, AC073218.2, AC073218.3, RNU7-40P, SPINK1, IL23A, and CXCL8.

### Utilities of Biomarkers

Any of the biomarkers described herein, either taken alone or in combination (e.g., at least two biomarkers, at least three biomarkers, or more biomarkers), can be used in the assay methods also described herein for analyzing a sample from a subject that has or is at risk for preeclampsia. Results obtained from such assay methods can be used in either clinical applications or non-clinical applications, including, but not limited to, those described herein.

(i) Analysis of Biological Samples

Any sample that may contain a biomarker (e.g., a biological sample such as endometrial tissue, endometrial cells, or endometrial fluid) can be analyzed by the assay methods described herein. The methods described herein may include providing a sample obtained from a subject. In some examples, the sample may be from an in vitro assay, for example, an in vitro cell culture (e.g., an in vitro culture of human endometrial stromal cells (hESCs)). As used herein, a “sample” refers to a composition that comprises biological materials such as (but not limited to) endometrial tissue, endometrial cells, or endometrial fluid from a subject. A sample includes both an initial unprocessed sample taken from a subject as well as subsequently processed, e.g., partially purified or preserved forms. Exemplary samples include endometrial tissue, endometrial stromal cells, placental tissue, fetal tissue, blood, plasma, or mucus. Exemplary endometrial tissue includes, but is not limited to, decidua basalis, decidua capsularis, or decidua parietalis. In some embodiments, the sample is a body fluid sample such as an endometrial fluid sample. In some embodiments, multiple (e.g., at least 2, 3, 4, 5, or more) samples may be collected from subject, over time or at particular time intervals, for example to assess the disease progression or evaluate the efficacy of a treatment.

A sample can be obtained from a subject using any means known in the art. In some embodiments, the sample is obtained from the subject by removing the sample (e.g., an endometrial tissue sample) from the subject. In some embodiments, the sample is obtained from the subject by a surgical procedure (e.g., dilation and curettage (D&C)). In some embodiments, the sample is obtained from the subject by a biopsy (e.g., an endometrial biopsy). In some embodiments, the sample is obtained from the subject by aspirating, brushing, scraping, or a combination thereof. In some embodiments, the sample is obtained from the subject after labor and delivery. In some embodiments, the sample is obtained from a human.

The term “subject” refers to a subject in need of the analysis described herein. In some embodiments, the subject is a patient. In some embodiments, the subject is a human. In some embodiments, the subject is a female human (a woman). In some embodiments, a subject is a woman who was previously pregnant. In some embodiments, a subject is a woman who previously had preeclampsia (e.g., during a prior pregnancy). In some embodiments, the human is pregnant or trying to become pregnant (e.g., with a first or subsequent pregnancy). In some embodiments, a subject is a pregnant woman (e.g., with a first or subsequent pregnancy). In some embodiments, a subject is at risk for preeclampsia (whether known or unknown). Such a subject may exhibit one or more risk factors associated with preeclampsia. Exemplary risk factors include, but are not limited to, a pregnancy with more than one baby, a history of chronic high blood pressure, diabetes, kidney disease or organ transplantation, a first time pregnancy, obesity, maternal age over 40, maternal age under 18, a family history of preeclampsia, polycystic ovarian syndrome, a subject who has one or more autoimmune disorders (e.g., lupus), a previous history of in vitro fertilization, or sickle cell disease. A subject may also include a person undergoing fertility treatment (e.g., in vitro fertilization or related procedures).

Alternatively, the subject in need of the analysis described herein may be a patient who has or is at risk for preeclampsia (known or unknown). Such a subject may currently have preeclampsia, or may have had preeclampsia in the past. Such a subject may be at risk for preeclampsia. In some examples, the subject is a human patient who is being treated for preeclampsia with, for example, an antihypertensive agent, an anticoagulant, a corticosteroid, an anticonvulsant, an antioxidant, a glycosaminoglycan, bed rest, hospitalization, maternal and fetal monitoring, and/or delivery. In other instances, such a human patient may be free of such a treatment (e.g., is not being treated currently). In some embodiments, treatment is initiated in a subject after identifying the subject as being at risk for preeclampsia.

Examples of preeclampsia include, without limitation, mild preeclampsia, severe preeclampsia (sPE), eclampsia, and HELLP (hemolysis, elevated liver enzymes, low platelet count) syndrome.

Any of the samples described herein can be subject to analysis using the assay methods described herein, which involve measuring the level of one or more biomarkers as described herein. Levels (e.g., the amount) of a biomarker disclosed herein, or changes in levels the biomarker, can be assessed using conventional assays or those described herein.

As used herein, the terms “determining” or “measuring,” or alternatively “detecting,” may include assessing the presence, absence, quantity and/or amount (which can be an effective amount) of a substance within a sample, including the derivation of qualitative or quantitative concentration levels of such substances, or otherwise evaluating the values and/or categorization of such substances in a sample from a subject.

In some embodiments, the level of a biomarker is assessed or measured by directly detecting the protein in a sample (e.g., an endometrial tissue sample, endometrial cell sample, or endometrial fluid sample). Alternatively or in addition, the level of a protein can be assessed or measured indirectly in a sample, for example, by detecting the level of activity of the protein (e.g., enzymatic assay).

The level of a protein (e.g., a biomarker protein) may be measured using an immunoassay. Examples of immunoassays include any known assay (without limitation), and may include any of the following: immunoblotting assay (e.g., Western blot), immunohistochemical analysis, flow cytometry assay, immunofluorescence assay (IF), enzyme linked immunosorbent assays (ELISAs) (e.g., sandwich ELISAs), radioimmunoassays, electrochemiluminescence-based detection assays, magnetic immunoassays, lateral flow assays, and related techniques. Additional suitable immunoassays for detecting a biomarker protein provided herein will be apparent to those of skill in the art.

Such immunoassays may involve the use of an agent (e.g., an antibody) specific to the target biomarker. An agent such as an antibody that “specifically binds” to a target biomarker is a term well understood in the art, and methods to determine such specific binding are also well known in the art. An antibody is said to exhibit “specific binding” if it reacts or associates more frequently, more rapidly, with greater duration and/or with greater affinity with a particular target biomarker than it does with alternative biomarkers. It is also understood by reading this definition that, for example, an antibody that specifically binds to a first target peptide may or may not specifically or preferentially bind to a second target peptide. As such, “specific binding” or “preferential binding” does not necessarily require (although it can include) exclusive binding. Generally, but not necessarily, reference to binding means preferential binding. In some examples, an antibody that “specifically binds” to a target peptide or an epitope thereof may not bind to other peptides or other epitopes in the same antigen. In some embodiments, a sample may be contacted, simultaneously or sequentially, with more than one binding agent that binds different protein biomarkers (e.g., multiplexed analysis).

As used herein, the term “antibody” refers to a protein that includes at least one immunoglobulin variable domain or immunoglobulin variable domain sequence. For example, an antibody can include a heavy (H) chain variable region (abbreviated herein as V_{H}), and a light (L) chain variable region (abbreviated herein as V_{L}). In another example, an antibody includes two heavy (H) chain variable regions and two light (L) chain variable regions. The term “antibody” encompasses antigen-binding fragments of antibodies (e.g., single chain antibodies, Fab and sFab fragments, F(ab')2, Fd fragments, Fv fragments, scFv, and domain antibodies (dAb) fragments (de Wildt et al., Eur J Immunol. 1996; 26(3):629-39.)) as well as complete antibodies. An antibody can have the structural features of IgA, IgG, IgE, IgD, IgM (as well as subtypes thereof). Antibodies may be from any source including, but not limited to, primate (human and non-human primate) and primatized (such as humanized) antibodies.

In some embodiments, the antibodies as described herein can be conjugated to a detectable label and the binding of the detection reagent to the peptide of interest can be determined based on the intensity of the signal released from the detectable label. Alternatively, a secondary antibody specific to the detection reagent can be used. One or more antibodies may be coupled to a detectable label. Any suitable label known in the art can be used in the assay methods described herein. In some embodiments, a detectable label comprises a fluorophore. As used herein, the term “fluorophore” (also referred to as “fluorescent label” or “fluorescent dye”) refers to moieties that absorb light energy at a defined excitation wavelength and emit light energy at a different wavelength. In some embodiments, a detection moiety is or comprises an enzyme. In some embodiments, an enzyme is one (e.g., (3-galactosidase) that produces a colored product from a colorless substrate.

In some examples, an assay method described herein is applied to measure the level of a cellular biomarker in a sample. Such cells may be collected according to routine practice and the level of cellular biomarkers can be measured via a conventional method.

In other examples, an assay method described herein is applied to measure the level of a circulate biomarker in a sample, which can be any biological sample including, but not limited to, a fluid sample (e.g., a blood sample or plasma sample), a tissue sample, or a cell sample. Any of the assays known in the art including, e.g., immunoassays can be used for measuring the level of such biomarkers.

It will be apparent to those of skill in the art that this disclosure is not limited to immunoassays. Detection assays that are not based on an antibody, such as mass spectrometry, are also useful for the detection and/or quantification of biomarkers as provided herein. Assays that rely on a chromogenic substrate can also be useful for the detection and/or quantification of biomarkers as provided herein.

Alternatively, the level of nucleic acids encoding a biomarker in a sample can be measured via a conventional method. In some embodiments, measuring the expression level of nucleic acid encoding the biomarker comprises measuring mRNA. In some embodiments, the expression level of mRNA encoding a biomarker can be measured using real-time reverse transcriptase (RT) Q-PCR or a nucleic acid microarray. Methods to detect biomarker nucleic acid sequences include, but are not limited to, polymerase chain reaction (PCR), reverse transcriptase-PCR (RT-PCR), in situ PCR, quantitative PCR (Q-PCR), real-time quantitative PCR (RT Q-PCR), in situ hybridization, Southern blot, Northern blot, sequence analysis, microarray analysis, detection of a reporter gene, or other DNA/RNA hybridization platforms.

In some embodiments, the level of nucleic acids encoding a biomarker in a sample can be measured via a hybridization assay. In some embodiments, the hybridization assay comprises at least one binding partner. In some embodiments, the hybridization assay comprises at least one oligonucleotide binding partner. In some embodiments, the hybridization assay comprises at least one labeled oligonucleotide binding partner. In some embodiments, the hybridization assay comprises at least one pair of oligonucleotide binding partners. In some embodiments, the hybridization assay comprises at least one pair of labeled oligonucleotide binding partners.

In some embodiments, the hybridization assay comprises at least one oligonucleotide binding partner set forth as any one of SEQ ID NOs.:1-8. In some embodiments, the hybridization assay comprises a pair of oligonucleotide binding partners set forth as SEQ ID NO.:1 and SEQ ID NO.:2. In some embodiments, the hybridization assay comprises a pair of oligonucleotide binding partners set forth as SEQ ID NO.:3 and SEQ ID NO.:4. In some embodiments, the hybridization assay comprises a pair of oligonucleotide binding partners set forth as SEQ ID NO.:5 and SEQ ID NO.:6. In some embodiments, the hybridization assay comprises a pair of oligonucleotide binding partners set forth as SEQ ID NO.:7 and SEQ ID NO.:8. In some embodiments, a label can be a fluorescent label, a radiolabel, or other detectable label as described herein.

Any binding agent that specifically binds to a desired biomarker may be used in the methods and kits described herein to measure the level of a biomarker in a sample. In some embodiments, the binding agent is an antibody or an aptamer that specifically binds to a desired protein biomarker. In other embodiments, the binding agent may be one or more oligonucleotides complementary to a coding nucleic acid or a portion thereof. In some embodiments, a sample may be contacted, simultaneously or sequentially, with more than one binding agent that binds different biomarkers (e.g., multiplexed analysis).

To measure the level of a target biomarker, a sample can be in contact with a binding agent under suitable conditions. In general, the term “contact” refers to an exposure of the binding agent with the sample or cells collected therefrom for suitable period sufficient for the formation of complexes between the binding agent and the target biomarker in the sample, if any. In some embodiments, the contacting is performed by capillary action in which a sample is moved across a surface of the support membrane.

In some embodiments, the assays may be performed on low-throughput platforms, including single assay format. For example, a low throughput platform may be used to measure the presence and amount of a protein in a sample (e.g., endometrium tissue, endometrial stromal cells, and/or endometrial fluid) for diagnostic methods, monitoring of disease and/or treatment progression, and/or predicting whether a disease or disorder may benefit from a particular treatment.

In some embodiments, it may be necessary to immobilize a binding agent to the support member. Methods for immobilizing a binding agent will depend on factors such as the nature of the binding agent and the material of the support member and may require particular buffers. Such methods will be evident to one of ordinary skill in the art. For example, the biomarker set in a sample as described herein may be measured using any of the kits and/or detecting devices which are also described herein.

The type of detection assay used for the detection and/or quantification of a biomarker such as those provided herein may depend on the particular situation in which the assay is to be used (e.g., clinical or research applications), on the kind and number of biomarkers to be detected, and/or on the kind and number of patient samples to be run in parallel, to name a few parameters.

The assay methods described herein may be used for both clinical and non-clinical purposes. Some examples are provided herein.

(ii) Diagnostic and/or Prognostic Applications

The levels of one or more of the biomarkers in a sample obtained from a subject may be measured by the assay methods described herein and used for various clinical purposes. These clinical purposes may include, but are not limited to: identifying a subject having preeclampsia, identifying a subject at risk for developing preeclampsia, monitoring the progress of preeclampsia in a subject, assessing the efficacy of a treatment for preeclampsia, identifying patients suitable for a particular treatment, and/or predicting preeclampsia relapse in a subject. Accordingly, described herein are diagnostic and prognostic methods for preeclampsia, (e.g., severe preeclampsia (sPE)), based on the level of one or more biomarkers described herein.

When needed, the level of a biomarker in a sample as determined by an assay methods described herein may be normalized with an internal control in the same sample or with a standard sample (having a predetermined amount of the biomarker) to obtain a normalized value. Either the raw value or the normalized value of the biomarker can then be compared with that in a reference sample or a control sample. A deviated (e.g., increased or reduced) value of the biomarker in a sample obtained from a subject as relative to the value of the same biomarker in the reference or control sample is indicative of preeclampsia in the sample. Such a sample indicates that the subject from which the sample was obtained may have or be at risk for preeclampsia.

In some embodiments, the level of the biomarker in a sample obtained from a subject can be compared to a predetermined threshold value for that biomarker, and a deviated (e.g., elevated or reduced) value of the biomarker may indicate that the subject has or is at risk for preeclampsia.

The control sample or reference sample may be a sample obtained from a healthy individual. Alternatively, the control sample or reference sample contains a known amount of the biomarker to be assessed. In some embodiments, the control sample or reference sample is a sample obtained from a control subject.

In some embodiments, the control subject is a pregnant individual having a complication free pregnancy. In some embodiments, the control subject is a non-pregnant individual with at least one previous normal pregnancy outcome. In some embodiments, the control subject is a non-pregnant individual with at least one previous pregnancy complicated by preterm birth with no signs of infection (non-infected preterm birth, nPTB). In some embodiments, the control subject is a non-pregnant individual with at least one previous preeclampsia pregnancy outcome.

As used herein, a control subject may be a healthy individual, i.e., an individual that is apparently free of preeclampsia at the time the level of the protein(s) is measured or has no history of the disease. A control subject may also represent a population of healthy subjects, who preferably would have one or more matching features (e.g., age, gestational age, ethnic group, pregnancy status) when compared to the subject being analyzed by a method described herein.

The control level can be a predetermined level or threshold. Such a predetermined level can represent the level of the protein in a population of subjects that do not have or are not at risk for preeclampsia (e.g., the average level in the population of healthy subjects). It can also represent the level of the protein in a population of subjects that have preeclampsia.

The predetermined level can take a variety of forms. For example, it can be single cut-off value, such as a median or mean. In some embodiments, such a predetermined level can be established based upon comparative groups, such as where one defined group is known to have preeclampsia and another defined group is known to not have preeclampsia. Alternatively, the predetermined level can be a range including, for example, a range representing the levels of the protein in a control population.

The control level as described herein can be determined by any technology known in the field. In some examples, the control level can be obtained by performing a conventional method (e.g., the same assay for obtaining the level of the protein in a test sample as described herein) on a control sample as also described herein. In other examples, levels of the protein can be obtained from members of a control population and the results can be analyzed by any method known in the field (e.g., a computational program) to obtain the control level (a predetermined level) that represents the level of the protein in the control population.

By comparing the level of a biomarker in a sample obtained from a candidate subject to the reference value as described herein, it can be determined whether the candidate subject has or is at risk for preeclampsia (e.g., severe preeclampsia (sPE)). For example, if the level of biomarker(s) in a sample from the candidate subject deviates (e.g., is increased or decreased) from the reference value (by e.g., 1%, 5%, 10%, 20%, 30%, 40%, 50%, 60%, 70%, 80%, 90%, 100%, 150%, 200%, 300%, 400%, 500% or more from a reference value), the candidate subject might be identified as having or at risk for preeclampsia. When the reference value represents the value range of the level of the biomarker in a population of subjects having or at risk for preeclampsia, the value of biomarker in a sample of a candidate falling in the range indicates that the candidate subject has or is at risk for preeclampsia.

As used herein, “an absolute value of the ratio” refers to the ratio of the determined level of the biomarker in the sample to the control level of the biomarker. Control levels are described in detail herein. In some embodiments, the absolute value of the ratio is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 150, at least 200, at least 300, at least 400, at least 500, or at least 1000. In some embodiments, the absolute value of the ratio is between 2-1000. In some embodiments, the absolute value of the ratio is between 5-1000, between 10-1000, between 15-1000, between 20-1000, between 30-1000, between 40-1000, between 50-1000, between 60-1000, between 70-1000, between 80-1000, between 90-100, between 100-1000, between 200-1000, between 300-1000, between 400-1000, or between 500-1000. In some embodiments, the absolute value of the ratio is between 2-500, between 2-400, between 2-300, between 2-200, between 2-100, between 2-90, between 2-80, between 2-70, between 2-60, between 2-50, between 2-40, between 2-30, between 2-20, between 2-15, between 2-10, or between 2-5.

As used herein, “an elevated level,” “an increased level,” or “a level above a reference value” means that the level of the biomarker is higher than a reference value, such as a predetermined threshold of a level the biomarker in a control sample. An elevated or increased level of a biomarker includes a level of the biomarker that is, for example, 1%, 5%, 10%, 20%, 30%, 40%, 50%, 60%, 70%, 80%, 90%, 100%, 150%, 200%, 300%, 400%, 500% or more above a reference value. In some embodiments, the level of the biomarker in the test sample is at least 1.1., 1.2, 1.3, 1.4, 1.5, 1.6, 1.7, 1.8, 1.9, 2, 2.5, 3, 3.5, 4, 4.5, 5, 6, 7, 8, 9, 10, 25, 50, 100, 150, 200, 300, 400, 500, 1000, 10000-fold or more higher than the level of the biomarker in a reference sample.

As used herein, “a reduced level,” “a decreased level,” or “a level below a reference value” means that the level of the biomarker is lower than a reference value, such as a predetermined threshold of a level the biomarker in a control sample. A reduced or decreased level of a biomarker includes a level of the biomarker that is, for example, 1%, 5%, 10%, 20%, 30%, 40%, 50%, 60%, 70%, 80%, 90%, 100%, 150%, 200%, 300%, 400%, 500% or more below a reference value. In some embodiments, the level of the biomarker in the test sample is at least 1.1, 1.2, 1.3, 1.4, 1.5, 1.6, 1.7, 1.8, 1.9, 2, 2.5, 3, 3.5, 4, 4.5, 5, 6, 7, 8, 9, 10, 25, 50, 100, 150, 200, 300, 400, 500, 1000, 10000-fold or more less than the level of the biomarker in a reference sample.

In some embodiments, the candidate subject is a human patient having a symptom of preeclampsia. For example, the subject may have one or more of the following symptoms or a combination thereof: proteinuria, kidney problems, headaches, changes in vision, abdominal pain, nausea or vomiting, decreased urine output, thrombocytopenia, impaired liver function, shortness of breath. In other embodiments, the subject has no symptoms or appears to have no symptoms of preeclampsia at the time the sample is collected, has no history of a symptom of preeclampsia, or no history of preeclampsia. In yet other embodiments, the subject is pregnant or trying to become pregnant.

A subject identified in the methods described herein as having or at risk for having preeclampsia may be subject to a suitable treatment, such as treatment with an antihypertensive agent, an anticoagulant, a corticosteroid, an anticonvulsant, an antioxidant, a glycosaminoglycan, bed rest, hospitalization, maternal and fetal monitoring, and/or delivery, as described herein.

The assay methods and kits described herein also can be used to evaluate the efficacy of a treatment for preeclampsia, such as those described herein, given the relationship that was established between the level of the biomarkers and preeclampsia. For example, multiple samples (e.g., endometrial tissue samples, endometrial fluid samples, or endometrial cell samples) can be collected from a subject to whom a treatment is performed either before and after the treatment or during the course of the treatment. The levels of a biomarker can be measured by any of the assay methods or devices described herein and values (e.g., amounts) of a biomarker can be determined accordingly. For example, if the absolute value of a biomarker indicates that a subject has preeclampsia and the level of the biomarker changes after the treatment or over the course of the treatment (e.g., in a later collected sample when compared to an earlier collected sample), it indicates that the treatment is effective. In some examples, the treatment involves an effective amount of an anti-preeclampsia therapy. Examples of anti-preeclampsia therapies include, but are not limited to, an antihypertensive agent, an anticoagulant, a corticosteroid, an anticonvulsant, an antioxidant, a glycosaminoglycan, bed rest, hospitalization, maternal and fetal monitoring, and delivery.

If the subject is identified as not responsive to the treatment, a higher dose and/or frequency of dosage of the therapeutic agent (e.g., an antihypertensive agent, an anticoagulant, a corticosteroid, an anticonvulsant, an antioxidant, and/or a glycosaminoglycan) are administered to the subject identified) may be administered or an alternative treatment may be administered. In some embodiments, the dosage or frequency of dosage of the therapeutic agent is maintained, lowered, or ceased in a subject identified as responsive to the treatment or not in need of further treatment. Alternatively, an alternative treatment can be administered to a subject who is found to not be responsive to a first or subsequent treatment. In some embodiments, an alternative treatment can be administered to a subject who is found to have a negative reaction to a first or subsequent treatment.

In other embodiments, the values of a biomarker or biomarker set can also be used to identify a preeclampsia that may be treatable using, for example, an antihypertensive agent, an anticoagulant, a corticosteroid, an anticonvulsant, an antioxidant, a glycosaminoglycan, bed rest, hospitalization, maternal and fetal monitoring, and/or delivery. To practice this method, the level of a biomarker in a sample collected from a subject (e.g., a endometrium tissue sample) having preeclampsia can be measured by a suitable method (e.g., those described herein such as a Western blot or a RT Q-PCR assay). If the level of the biomarker is elevated or reduced from the reference value, it indicates that an anti-preeclampsia treatment may be effective in treating the disease. If the disease is identified as being susceptible to treatment with an anti-preeclampsia therapy (e.g., one or more symptoms of preeclampsia may be improved or cease), the method may further comprise administering to the subject having the disease an effective amount of an anti-preeclampsia therapy.

In other embodiments, the values of a biomarker or biomarker set can be relied on to evaluate the severity of preeclampsia. For example, as described herein, preeclampsia may be in the mild state, during which the subject does not experience symptoms of the disease. In another example, preeclampsia may be severe preeclampsia (sPE), during which the subject has severe symptoms, such as impaired liver function. In some embodiments, the level of one or more biomarkers is indicative of whether the subject will experience, is experiencing, or will soon experience preeclampsia (e.g., severe preeclampsia (sPE)). In some embodiments, the methods involve comparing the level of a biomarker in a sample obtained from a subject having preeclampsia to the level of the biomarker in a control sample from the same subject, for example a sample obtained from the same subject prior to pregnancy or a sample obtained from the same subject during a complication free (e.g., without preeclampsia) pregnancy.

Also within the scope of the present disclosure are methods of evaluating a subject for transfer of one or more fertilized eggs or embryos. To practice this method, the level of a biomarker in a sample collected from a subject trying to become pregnant and at risk for preeclampsia can be measured by a suitable method. If the biomarker level or levels indicate that the subject is likely to not suffer from preeclampsia, one or more fertilized eggs or embryos may be transferred to the subject. If the biomarker level or levels indicate that the subject is likely to or will suffer from preeclampsia, one or more fertilized eggs or embryos may be transferred to the subject before, after, or concurrently with one or more treatments for preeclampsia. A fertilized egg or embryo can be transferred to a subject using any means known in the art including, but not limited to, in vitro fertilization (IVF), ultra-sound guided IVF, and surgical embryo transfer (SET).

(iii) Non-Clinical Applications

Further, levels of any of the biomarkers described herein may be applied for non-clinical uses including, for example, for research purposes. In some embodiments, the methods described herein may be used to study cell behavior and/or cell mechanisms. For example, one or more of the biomarkers described herein may be used to evaluate decidualization, which can be used for various purposes, including studies on decidualization and development of new agents that specifically target decidualization defects.

In some embodiments, the levels of biomarker sets, as described herein, may be relied on in the development of new therapeutics for preeclampsia. For example, the levels of a biomarker may be measured in samples obtained from a subject who has been administered a new therapy (e.g., a clinical trial). In some embodiments, the level of the biomarker set may indicate the efficacy of the new therapeutic or the progression of preeclampsia in the subject prior to, during, or after the administration of the new therapy.

### Kits and Detecting Devices for Measuring Biomarkers

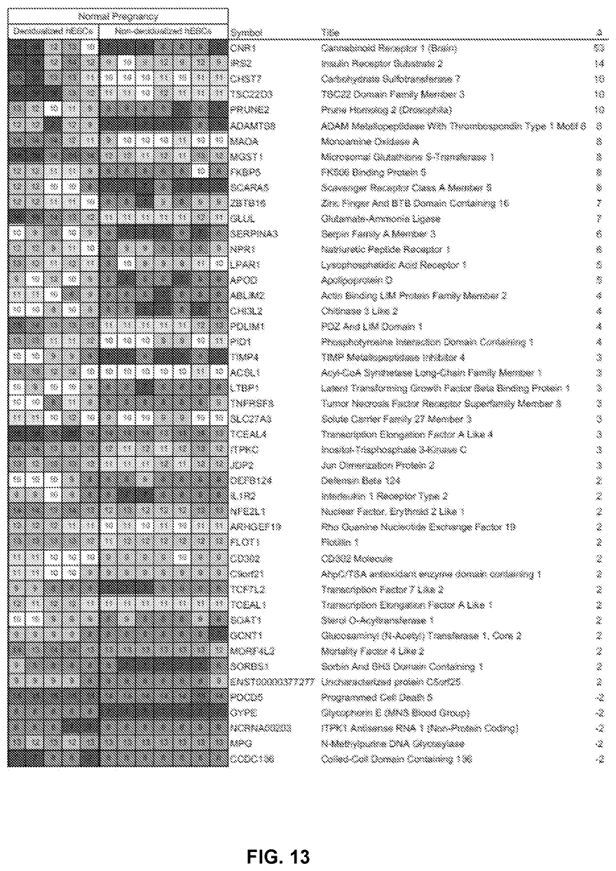

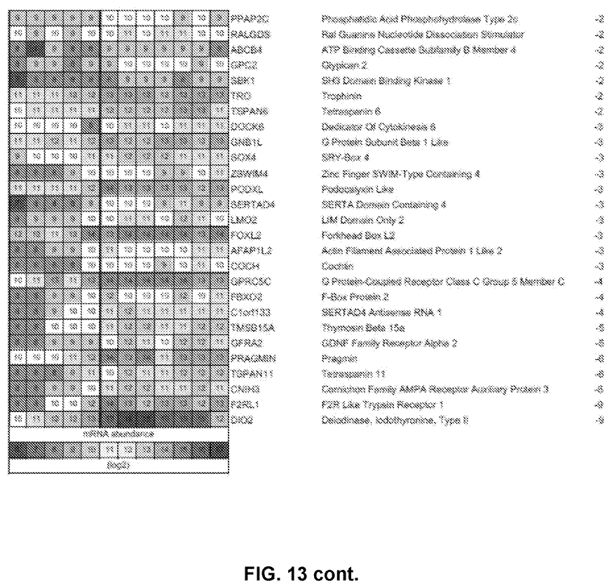

The present disclosure also provides kits and devices for use in measuring the level of a biomarker set as described herein. Such a kit or device can comprise one or more binding agents (e.g., oligonucleotides, antibodies, etc.) that specifically bind to a gene product of target biomarkers, such as the biomarkers listed in FIGS. 13-16, FIG. 18, Tables 1, A, B, and/or C, and/or subsets thereof. For example, such a kit or detecting device may comprise at least one binding agent that is specific to one or more transcripts (e.g., mRNA) or protein biomarkers expressed from the genes selected from FIGS. 13-16, FIG. 18, Tables 1, A, B, and/or C, and/or subsets thereof. In some instances, the kit or detecting device comprises binding agents specific to two or more members of the RNA and/or protein biomarker sets described herein.

Levels of specific expression products of genes (e.g., ADAMTS8, CHI3L2, CHST7, CNR1, COCH, FBXO2, NPR1, SCARA5, and/or SERPINA3, and/or any one or more of the differentially expressed genes listed in Table 1, A, B, and/or C) can be assessed by any appropriate method. In some embodiments, the levels of specific expression products are analyzed using one or more assays comprising any solid support (e.g., one or more chips). For example, a solid support (e.g., a chip) may be used to analyze at least one (e.g., 1, 2, 3, 4, 5, 6, 7, 8, 9, 10, or more) biological sample(s) of or from a subject. Accordingly, in some embodiments a kit comprises a plurality of gene specific polynucleotide probes or primers that can be used in a hybridization and/or sequencing assay (e.g., a next generation sequencing reaction). In some embodiments, a kit comprises one or more other binding agents (e.g., aptamers, antibodies, and/or other binding agents). In some embodiments, different binding agents are provided in separate containers (e.g., in a dry powder, solution, suspension, or other form). In some embodiments, mixtures of two or more binding agents (e.g., 2 or more polynucleotide probes or primers) are provided (e.g., in a dry powder, solution, suspension, or other form). In some embodiments, one or more binding agents are provided attached to a solid support (e.g., a chip, for example in the form of an array of probes, primers, or antibodies). In some embodiments, one or more binding agents are labeled (e.g., with a fluorescent, luminescent, radioactive, enzyme linker, or other detectable marker).

Sections of the solid support (e.g., the chip) may be modified with one binding partner or more than one binding partner. The solid support may be linked in any manner to the binding partner(s). As a non-limiting example, the binding partner(s) may be physisorbed or otherwise bound (e.g., bound directly) onto the surface of the solid support or covalently linked through appropriate coupling chemistry in any manner including, but not limited to: linkage through a epoxide on the surface, creation of an amido link (e.g., through NHS EDC chemistry) using a amine or carboxylic acid group present on the surface, linkage between a thiol and a thiol reactive group (e.g., a maleimide group), formation of a Schiff base between aldehyde and amines, reaction to an anhydride present on the surface, and/or through a photo-activatable linker.

The binding partner may be any binding partner useful for the instant compositions or methods. For example, the binding partner may be a protein (with naturally occurring amino acids or artificial amino acids), one or more nucleic acids made of naturally occurring bases or artificial bases (including, for example, DNA or RNA), sugars, carbohydrates, one or more small molecules (including, but not limited to one or more of: a vitamin, hormone, cofactor, heme group, chelate, fatty acid, or other known small molecule, and/or a phage).

The binding partners may be applied to the surface of the substrate by deposition of a droplet at a pre-defined location in any manner and using any device including, but not limiting to: the use of a pipette, a liquid dispenser, plotter, nano-spotter, nano-plotter, arrayer, spraying mechanism or other suitable fluid handling device.

In some embodiments, antibodies or antigen-binding fragments are provided that are suited for use in the instant methods and compositions. Immunoassays utilizing such antibody or antigen-binding fragments useful for the instant compositions and methods may be competitive or non-competitive immunoassays in either a direct or an indirect format. Non-limiting examples of such immunoassays are Enzyme Linked Immunoassays (ELISA), radioimmunoassays (RIA), sandwich assays (immunometric assays), flow cytometry-based assays, western blot assays, immunoprecipitation assays, immunohistochemistry assays, immuno-microscopy assays, lateral flow immuno-chromatographic assays, and proteomics arrays. For example, the binding partners may be antibodies (or antibody-binding fragments thereof) with specificity towards a protein of interest including one or more of ADAMTS8, CHI3L2, CHST7, CNR1, COCH, FBXO2, NPR1, SCARA5, and/or SERPINA3.

In some embodiments, oligonucleotide binding partners are used to assess the levels of specific expression products of genes. The oligonucleotide binding partners may be of any type known or used. As a set of non-limiting examples, in certain embodiments the oligonucleotide probes may be RNA oligonucleotides, DNA oligonucleotides, a mixture of RNA oligonucleotides and DNA nucleotides, and/or oligonucleotides that may be mixtures of RNA and DNA. The oligonucleotide binding partners may be naturally occurring or synthetic. The oligonucleotide binding partners may be of any length. As a set of non-limiting examples, the length of the oligonucleotide binding partners may range from about 5 to about 50 nucleotides, from about 10 to about 40 nucleotides, or from about 15 to about 40 nucleotides. The array may comprise any number of oligonucleotide binding partners specific for each target gene. For example, the array may comprise less than 10 (e.g., 9, 8, 7, 6, 5, 4, 3, 2, or 1) oligonucleotide probes specific for each target gene. As another example, the array may comprise more than 10, more than 50, more than 100, or more than 1000 oligonucleotide binding partners specific for each target gene.

The array may further comprise control binding partners such as, for example mismatch control oligonucleotide binding partners or control antibodies or antigen binding fragments thereof. Where mismatch control oligonucleotide binding partners are present, the quantifying step may comprise calculating the difference in hybridization signal intensity between each of the oligonucleotide binding partners and its corresponding mismatch control binding partner. Where control antibodies or antigen binding fragments thereof are present, the quantifying step may comprise calculating the difference in hybridization signal intensity between antibodies or antigen binding fragments for the genes under examination (e.g., ADAMTS8, CHI3L2, CHST7, CNR1, COCH, FBXO2, NPR1, SCARA5, and/or SERPINA3) and a control or “housekeeping” antibody or antigen binding fragment thereof. The quantifying may further comprise calculating the average difference in hybridization signal intensity between each of the oligonucleotide probes and its corresponding mismatch control probe for each gene.

The array (e.g., chip) may contain any number of analysis regions. As a set of non-limiting examples, the array may contain one or more than one (e.g., 2, 3, 4, 5, 6, 7, 8, 9, 10, 20, 25, 30, 35, 40, or more) analysis regions. Each analysis region may comprise any number of binding partners immobilized to a substrate portion therein. As a non-limiting set of examples, each analysis region may comprise between one and 1,000 binding partners, one and 500 binding partners, one and 250 binding partners, one and 100 binding partners, two and 1,000 binding partners, two and 500 binding partners, two and 250 binding partners, two and 100 binding partners, three and 1,000 binding partners, three and 500 binding partners, three and 250 binding partners, or three and 100 binding partners immobilized to a substrate portion therein.

Binding partners including, but not limited to, antibodies or antigen-binding fragments that bind to the specific antigens of interest can be immobilized, e.g., by binding to a solid support (e.g., a chip, carrier, membrane, columns, proteomics array, etc.). In one set of embodiments, a material used to form the solid support has an optical transmission of greater than 90% between 400 and 800 nm wavelengths of light (e.g., light in the visible range). Optical transmission may be measured through a material having a thickness of, for example, about 2 mm (or in other embodiments, about 1 mm or about 0.1 mm). In some instances, the optical transmission is greater than or equal to 80%, greater than or equal to 85%, greater than or equal to 88%, greater than or equal to 92%, greater than or equal to 94%, or greater than or equal to 96% between 400 and 800 nm wavelengths of light. In some embodiments, the material used to form the solid support has an optical transmission of less than or equal to 99.9%, less than or equal to 96%, less than or equal to 94%, less than or equal to 92%, less than or equal to 90%, less than or equal to 85%, less than or equal to 80%, less than or equal to 50%, less than or equal to 30%, or less than or equal to 10% between 400 and 800 nm wavelengths of light. Combinations of the above-referenced ranges are also possible.

The array may be fabricated on a surface of virtually any shape (e.g., the array may be planar) or even a multiplicity of surfaces. Non-limiting examples of solid support materials useful for the compositions and methods described herein may include glass, plastics, elastomeric materials, membranes, or other suitable materials for performing immunoassays. The solid support may be formed from one material, or it may be formed from two or more materials.

Specific solid support materials may include, but are not limited to: any type of glass (e.g., fused silica, borosilicate glass, Pyrex®, or Duran®). In one embodiment, the solid support is a glass chip. The solid support may also comprise a non-glass substrate (e.g., a plastic substrate) coated with a glass film dioxide produced by a process such as sputtering, oxidation of silicon, or through reaction of silane reagents. The glass surface may be further modified with functionalized silane reagents including, for example: amine-terminated silanes (aminopropyltriethoxy silane) and epoxide-terminated silanes (glycidoxypropyltrimethoxysilane).

Additional specific solid support materials may include, but are not limited to: thermoplastic polymers and may comprise one or more of: polystyrene, polycarbonate, polymethylmetacrylate, cyclic olefin copolymers, polyethylene, polypropylene, polyvinyl chloride, polyvinylidene difluoride, any fluoropolymers (e.g., polytetrafluoroethylene, also known as Teflon®), polylactic acid, poly(methyl methacrylate) (also known as PMMA or acrylic; e.g., Lucite®, Perspex®, and Plexiglas®), and acrylonitrile butadiene styrene.

Additional specific solid support materials may include, but are not limited to: one or more elastomeric materials including polysiloxanes (silicones such as polydimethylsiloxane) and rubbers (polyisoprene, polybutadiene, chloroprene, styrene-butadiene, nitrile rubber, polyether block amides, ethylene-vinyl acetate, epichlorohydrin rubber, isobutene-isoprene, nitrile, neoprene, ethylene-propylene, and hypalon).

Additional specific solid support materials may include, but are not limited to: one or more membrane substrates such as dextran, amyloses, nylon, Polyvinylidene fluoride (PVDF), fiberglass, and natural or modified celluloses (e.g., cellulose, nitrocellulose, CNBr-activated cellulose, and cellulose modified with polyacrylamides, agaroses, and/or magnetite). The nature of the support can be either fixed or suspended in a solution (e.g., beads).

In some embodiments, the material and dimensions (e.g., thickness) of a solid support (e.g., a chip) is substantially impermeable to water vapor. In some embodiments, a cover may also be present. In some embodiments, the cover is substantially impermeable to water vapor. For instance, a solid support (e.g., a chip) may include a cover comprising a material known to provide a high vapor barrier, such as metal foil, certain polymers, certain ceramics and combinations thereof. Examples of materials having low water vapor permeability are provided below. In other cases, the material is chosen based at least in part on the shape and/or configuration of the chip. For instance, certain materials can be used to form planar devices whereas other materials are more suitable for forming devices that are curved or irregularly shaped.

A material used to form all or portions of a section or component of any composition described herein may have, for example, a water vapor permeability of less than about 5.0 g.mm/m².d, less than about 4.0 g.mm/m².d, less than about 3.0 g.mm/m².d, less than about 2.0 g.mm/m².d, less than about 1.0 g.mm/m².d, less than about 0.5 g.mm/m².d, less than about 0.3 g.mm/m².d, less than about 0.1 g.mm/m².d, or less than about 0.05 g.mm/m².d. In some cases, the water vapor permeability may be, for example, between about 0.01 g.mm/m².d and about 2.0 g.mm/m².d, between about 0.01 g.mm/m².d and about 1.0 g.mm/m².d, between about 0.01 g.mm/m².d and about 0.4 g.mm/m².d, between about 0.01 g.mm/m².d and about 0.04 g.mm/m².d, or between about 0.01 g.mm/m².d and about 0.1 g.mm/m².d. The water vapor permeability may be measured at, for example, 40 ° C. at 90% relative humidity (RH). Combinations of materials with any of the aforementioned water vapor permeabilities may be used in the instant compositions or methods.

In some embodiments, the material and dimensions of a solid support (e.g., a chip) and/or cover may vary. For example, the chip may be configured to provide one or more regions (e.g., liquid containment regions). In certain embodiments, the chip may be configured to provide two or more regions (e.g., liquid containment regions). In certain embodiments, two or more of the regions are fluidically separated from other regions. In one embodiment, all of the regions are fluidically separated from other regions. In some embodiments, all of the regions are fluidically connected. The chip may comprise any number of liquid containment regions. As a non-limiting example, the chip may comprise one, two, three, four, five, six, seven, eight, nine, or ten liquid containment regions, each of which may be fluidically separated from one another. In other embodiments, the chip may comprise one, two, three, four, five, six, seven, eight, nine, or ten liquid containment regions that are fluidically connected to one another.

A solid support (e.g., a chip) described herein may have any suitable volume for carrying out an analysis such as a chemical and/or biological reaction or other process. The entire volume of the solid support may include, for example, any reagent storage areas, analysis regions, liquid containment regions, waste areas, as well as one or more identifiers. In some embodiments, small amounts of reagents and samples are used and the entire volume of the a liquid containment region is, for example, less than or equal to 10 mL, less than or equal to 5 mL, less than or equal to 1 mL, less than or equal to 500 μL, less than or equal to 250 μL, less than or equal to 100 μL, less than or equal to 50 μL, less than or equal to 25 μL, less than or equal to 10 μL, less than or equal to 5 μL, or less than or equal to 1 μL. In some embodiments, small amounts of reagents and samples are used and the entire volume of the a liquid containment region is, for example, at least 10 mL, at least 5 mL, at least 1 mL, at least 500 μL, at least 250 μL, at least 100 μL, at least 50 μL, at least 25 μL, at least 10 μL, at least 5μL, or at least 1μL. Combinations of the above-referenced values are also possible.

The length and/or width of the solid support (e.g., chip) may be, for example, less than or equal to 300 mm, less than or equal to 200 mm, less than or equal to 150 mm, less than or equal to 100 mm, less than or equal to 95 mm, less than or equal to 90 mm, less than or equal to 85 mm, less than or equal to 80 mm, less than or equal to 75 mm, less than or equal to 70 mm, less than or equal to 65 mm, less than or equal to 60 mm, less than or equal to 55 mm, less than or equal to 50 mm, less than or equal to 45 mm, less than or equal to 40 mm, less than or equal to 35 mm, less than or equal to 30 mm, less than or equal to 25 mm, or less than or equal to 20 mm.

In some embodiments, the length and/or width of the chip may be, for example, at least 300 mm, at least 200 mm, at least 150 mm, at least 100 mm, at least 95 mm, at least 90 mm, at least 85 mm, at least 80 mm, at least 75 mm, at least 70 mm, at least 65 mm, at least 60 mm, at least 55 mm, at least 50 mm, at least 45 mm, at least 40 mm, at least 35 mm, at least 30 mm, at least 25 mm, or at least 20 mm. Combinations of the above-referenced values are also possible. In some embodiments, the thickness of the solid support (e.g., chip) may be, for example, less than or equal to 5 mm, less than or equal to 3 mm, less than or equal to 2 mm, less than or equal to 1 mm, less than or equal to 0.9 mm, less than or equal to 0.8 mm, less than or equal to 0.7 mm, less than or equal to 0.5 mm, less than or equal to 0.4 mm, less than or equal to 0.3 mm, less than or equal to 0.2 mm, or less than or equal to 0.1 mm. In some embodiments, the thickness of the solid support (e.g., chip) may be, for example, at least 5 mm, at least 3 mm, at least 2 mm, at least 1 mm, at least 0.9 mm, at least 0.8 mm, at least 0.7 mm, at least 0.5 mm, at least 0.4 mm, at least 0.3 mm, at least 0.2 mm, or at least 0.1 mm. Combinations of the above-referenced values are also possible. One or more solid supports (e.g., chips) may be analyzed at the same time by any suitable device. An adapter may be used with the one or more solid supports (e.g., chips) in order to insert and securely hold them in the analyzer.

In some embodiments, the solid support (e.g., chip) includes one or more identifiers. Any method or type of identification may be used. For example, an identifier may be, but is not limited to, any type of label such as a bar code or an RFID tag. The identifier may include the name, patient number, social security number, or any other method of identification for a subject. The identifier may also be a randomized identifier of any type useful in a clinical setting.

It should be understood that the solid supports (e.g., chips) and their respective components described herein are exemplary and that other configurations and/or types of solid supports (e.g., chips) and components can be used with the systems and methods described herein.

The binding of a one or more binding partners (e.g., to detect the binding of a protein or other substance of interest including, but not limited to, antigen-bound antibody complexes) may be quantified by any method known in the art. The quantification may, for example, be performed by detection or interrogation of an active molecule bound to an antibody. In a multiplexed format, where more than one assay is being performed on a continuous area, the signals associated with each assay should be differentiable from the other assays. Any suitable strategy known in the art may be used including, but not limited to: (1) using a label with substantially non-overlapping spectral and/or electrochemical properties: (2) using a signal amplification chemistry that remains attached or deposited in close proximity to the tracer itself.

In some embodiments, labeled binding partners (e.g., antibodies or antigen binding fragments) may be used as tracers to detect binding (e.g., using antigen bound antibody complexes). Examples of the types of labels which may be useful for the instant methods and compositions include enzymes, radioisotopes, colloidal metals, fluorescent compounds, magnetic, chemiluminescent compounds, electrochemiluminescent groups, metal nanoparticles, and bioluminescent compounds. Radiolabeled binding partners (e.g., antibodies) may be prepared using any known method and may involve coupling a radioactive isotope such as ¹⁵³Eu, ³H, ³²P, ³⁵S, ⁵⁹Fe, or ¹²⁵1, which can then be detected by gamma counter, scintillation counter or by autoradiography. Binding partners (e.g., antibodies or antigen binding fragments) may alternatively be labeled with enzymes such as yeast alcohol dehydrogenase, horseradish peroxidase, alkaline phosphatase, and the like, then developed and detected spectrophotometrically or visually. The label may be used to react a chromogen into a detectable chromophore (including, for example, if the chromogen is a precipitating dye).

Suitable fluorescent labels may include, but are not limited to: fluorescein, fluorescein isothiocyanate, fluorescamine, rhodamine, Alexa Fluor® dyes (such as Alexa Fluor® 350, Alexa Fluor® 405, Alexa Fluor® 430, Alexa Fluor® 488, Alexa Fluor® 514, Alexa Fluor® 532, Alexa Fluor® 546, Alexa Fluor® 555, Alexa Fluor® 568, Alexa Fluor® 594, Alexa Fluor® 610, Alexa Fluor® 633, Alexa Fluor® 635, Alexa Fluor® 647, Alexa Fluor® 660, Alexa Fluor® 680, Alexa Fluor® 700, Alexa Fluor® 750, or Alexa Fluor® 790), cyanine dyes including, but not limited to: Cy2, Cy3, Cy3.5, Cy5, Cy5.5, Cy7, and Cy7.5, and the like. The labels may also be time-resolved fluorescent (TRF) atoms (e.g., Eu or Sr with appropriate ligands to enhance TRF yield). More than one fluorophore capable of producing a fluorescence resonance energy transfer (FRET) may also be used. Suitable chemiluminescent labels may include, but are not limited to: acridinium esters, luminol, imidazole, oxalate ester, luciferin, and any other similar labels.

Suitable electrochemiluminescent groups for use may include, as a non-limiting example: Ruthenium and similar groups. A metal nanoparticle may also be used as a label. The metal nanoparticle may be used to catalyze a metal enhancement reaction (such as gold colloid for silver enhancement).

Any of the labels described herein or known in the field may be linked to the tracer using covalent or non-covalent means. The label may be presented on or inside an object like a bead (including, for example, a plain bead, hollow bead, or bead with a ferromagnetic core), and the bead is then attached to the binding partner (e.g., an antibody or antigen-binding fragment thereof). The label may also be a nanoparticle including, but not limited to, an up-converting phosphorescent system, nanodot, quantum dot, nanorod, and/or nanowire. The label linked to the antibody may also be a nucleic acid, which might then be amplified (e.g., using PCR) before quantification by one or more of optical, electrical or electrochemical means.

In some embodiments, the binding partner is a oligonucleotide binding partner. In some embodiments, the oligonucleotide binding partner binds to a nucleic acid sequence of a biomarker. In some embodiments, the oligonucleotide binding partner is a labeled oligonucleotide binding partner. In some embodiments, the oligonucleotide binding partner is set forth as SEQ ID NO.:1. In some embodiments, the oligonucleotide binding partner is set forth as SEQ ID NO.:2. In some embodiments, the oligonucleotide binding partner is set forth as SEQ ID NO.:3. In some embodiments, the oligonucleotide binding partner is set forth as SEQ ID NO.:4. In some embodiments, the oligonucleotide binding partner is set forth as SEQ ID NO.:5. In some embodiments, the oligonucleotide binding partner is set forth as SEQ ID NO.:6. In some embodiments, the oligonucleotide binding partner is set forth as SEQ ID NO.:7. In some embodiments, the oligonucleotide binding partner is set forth as SEQ ID NO.:8.

In some embodiments, the binding partner is immobilized on the solid support prior to formation of binding complexes. In other embodiments, immobilization of the antibody and antigen-binding fragment is performed after formation of binding complexes.

In one embodiment, immunoassay methods disclosed herein comprise immobilizing binding partners (e.g., antibodies or antigen-binding fragments) to a solid support (e.g., a chip); applying a sample (e.g., an endometrial fluid sample) to the solid support under conditions that permit binding of the expression product of a biomarker (e.g., a protein) to one or more binding partners (e.g., one or more antibodies or antigen-binding fragments), if present in the sample; removing the excess sample from the solid support; detecting the bound complex (using, e.g., detectably labeled antibodies or antigen-binding fragments) under conditions that permit binding (e.g., of an expression product to the antigen-bound immobilized antibodies or antigen-binding fragments); washing the solid support and assaying for the label.

Reagents can be stored in or on a chip for various amounts of time. For example, a reagent may be stored for longer than 1 hour, longer than 6 hours, longer than 12 hours, longer than 1 day, longer than 1 week, longer than 1 month, longer than 3 months, longer than 6 months, longer than 1 year, or longer than 2 years. Optionally, the chip may be treated in a suitable manner in order to prolong storage. For instance, chips having stored reagents contained therein may be vacuum sealed, stored in a dark environment, and/or stored at low temperatures (e.g., below 4 ° C. or 0 ° C.). The length of storage depends on one or more factors such as the particular reagents used, the form of the stored reagents (e.g., wet or dry), the dimensions and materials used to form the substrate and cover layer(s), the method of adhering the substrate and cover layer(s), and how the chip is treated or stored as a whole. Storing of a reagent (e.g., a liquid or dry reagent) on a solid support material may involve covering and/or sealing the chip prior to use or during packaging.

Any solid state assay device described herein may be included in a kit. The kit may include any packaging useful for such devices. The kit may include instructions for use in any format or language. The kit may also direct the user to obtain further instructions from one or more locations (physical or electronic). The included instructions can comprise a description of how to use the components contained in the kit for measuring the level of a biomarker set (e.g., protein biomarker or nucleic acid biomarker) in a biological sample collected from a subject, such as a human patient. The instructions relating to the use of the kit generally include information as to the amount of each component and suitable conditions for performing the assay methods described herein.

The components in the kits may be in unit doses, bulk packages (e.g., multi-dose packages), or sub-unit doses. The kit can also comprise one or more buffers as described herein but not limited to a coating buffer, a blocking buffer, a wash buffer, and/or a stopping buffer.

The kits of this present disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an PCR machine, a nucleic acid array, or a flow cytometry system.

Kits may optionally provide additional components such as interpretive information, such as a control and/or standard or reference sample. Normally, the kit comprises a container and a label or package insert(s) on or associated with the container. In some embodiments, the present disclosure provides articles of manufacture comprising contents of the kits described above.

### Treatment of Preeclampsia

A subject having or at risk for preeclampsia, as identified using the methods described herein, may be treated with any appropriate anti-preeclampsia therapy. In some embodiments, provided methods include selecting a treatment for a subject based on the output of the described method, e.g., measuring the level of a biomarker set.

In some embodiments, the method comprises one or both of selecting or administering a therapy, e.g., an antihypertensive agent, an anticoagulant, a corticosteroid, an anticonvulsant, an antioxidant, a glycosaminoglycan, bed rest, hospitalization, maternal and fetal monitoring, and/or delivery, for administration to the subject based on the output of the assay, e.g., biomarker detection.

In some embodiments, the therapy comprises administering an antihypertensive agent. Examples of anti-hypertensive agents include, but are not limited to, centrally acting α2-adrenergic agonists (e.g., methyldopa or clonidine), peripherally acting adrenergic-receptor antagonists (e.g., labetalol or prazosin), calcium channel blockers (e.g., nifedipine or verapamil), vasodilators (e.g., hydralazine or sodium nitroprusside), and diuretics (e.g., thiazide diuretics such as chlorothiazide, chlorthalidone, hydrochlorothiazide, indapamide, and metolazone). In some embodiments, the therapy comprises administering an anticoagulant. Examples of anticoagulants include, but are not limited to, glycoprotein platelet inhibitors (e.g., abciximab, eptifibatide, tirofiban), platelet aggregation inhibitors (e.g., aspirin, cangrelor, cilostazol, clopidogrel, dipyridamole, prasugrel, ticlopidine, or ticagrelor) and protease-activated receptor-1 antagonists (e.g., vorapaxar).

In some embodiments, the therapy comprises administering a corticosteroid. Examples of corticosteroids include, but are not limited to, hydrocortisone, methylprednisolone, prednisolone, prednisone, triamcinolone, amcinonide, budesonide, desonide, fluocinolone acetonide, fluocinonide, halcinonide, triamcinolone acetonide, beclometasone, betamethasone, dexamethasone, fluocortolone, halometasone, mometasone, alclometasone dipropionate, betamethasone dipropionate, betamethasone valerate, clobetasol propionate, clobetasone butyrate, fluprednidene acetate, mometasone furoate, ciclesonide, cortisone acetate, hydrocortisone aceponate, hydrocortisone acetate, hydrocortisone buteprate, hydrocortisone butyrate, hydrocortisone valerate, prednicarbate, and tixocortol pivalate

In some embodiments, the therapy comprises administering an anticonvulsant. Examples of anticonvulsants include, but are not limited to, magnesium sulphate, paraldehyde, stiripentol, phenobarbital, primidone, methylphenobarbital, mephobarbital, barbexaclone, clobazam, clonazepam, clorazepate, diazepam, midazolam, lorazepam, nitrazepam, temazepam, nimetazepam, potassium bromide, felbamate, carbamazepine, oxcarbazepine, eslicarbazepine acetate, valproic acid, sodium valproate, divalproex sodium, vigabatrin, progabide, tiagabine, vigabatrin, progabide, topiramate, gabapentin, pregabalin, hydantoins, ethotoin, phenytoin, mephenytoin, fosphenytoin, oxazolidinedione, paramethadione, trimethadione, ethadione, propionate, beclamide, pyrimidinedione, pyrrolidine, brivaracetam, levetiracetam, seletracetam, succinimide, ethosuximide, phensuximide, mesuximide, sulfonamide, acetazolamide, sultiame, methazolamide, zonisamide, triazine, lamotrigine, pheneturide, phenacemide, valpromide, valnoctamide, and perampanel.

In some embodiments, the therapy comprises administering an antioxidant. Examples of antioxidants include, but are not limited to, vitamin C and vitamin E. In some embodiments, the therapy comprises administering a low dose aspirin. In some embodiments, the therapy comprises bed rest. In some embodiments, the therapy comprises hospitalization. In some embodiments, the therapy comprises maternal and fetal monitoring. In some embodiments, the therapy comprises delivery of the fetus.

In some embodiments, the therapy comprises administering a glycosaminoglycan. Examples of a glycosaminoglycan include, but are not limited to, low molecular weight heparin, heparin sulfate, chemically modified heparin or heparin sulfate, low molecular weight dermatan sulfates and mixtures thereof.

An effective amount of the preeclampsia therapy can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation, or topical routes.

“An effective amount” as used herein refers to the amount of each active agent required to confer therapeutic effect on the subject, either alone or in combination with one or more other active agents. Effective amounts vary, as recognized by those skilled in the art, depending on the particular condition being treated, the severity of the condition, the individual patient parameters including age, physical condition, size, weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

Empirical considerations such as the half-life of an agent will generally contribute to the determination of the dosage. Frequency of administration may be determined and adjusted over the course of therapy, and is generally, but not necessarily, based on treatment and/or suppression and/or amelioration and/or delay of preeclampsia. Alternatively, sustained continuous release formulations of therapeutic agent may be appropriate. Various formulations and devices for achieving sustained release are known in the art.

As used herein, the term “treating” refers to the application or administration of a composition including one or more active agents to a subject who has preeclampsia, a symptom of preeclampsia, and/or a predisposition toward preeclampsia, with the purpose to cure, heal, alleviate, relieve, alter, remedy, ameliorate, improve, or affect the disorder, the symptom of the disease, and/or the predisposition toward preeclampsia.

Alleviating preeclampsia includes delaying the development or progression of the disease, and/or reducing disease severity. Alleviating the disease does not necessarily require curative results.

As used therein, “delaying” the development of a disease (such as preeclampsia) means to defer, hinder, slow, retard, stabilize, and/or postpone progression of the disease. This delay can be of varying lengths of time, depending on the history of the disease and/or individuals being treated. A method that “delays” or alleviates the development of a disease and/or delays the onset of the disease is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of preeclampsia includes initial onset and/or recurrence.

In some embodiments, the therapy is administered one or more times to the subject. The therapy, e.g., an antihypertensive agent, an anticoagulant, a corticosteroid, an anticonvulsant, an antioxidant, a glycosaminoglycan, bed rest, hospitalization, maternal and fetal monitoring, and/or delivery, may be administered along with another therapy as part of a combination therapy for treatment of preeclampsia.

The term combination therapy, as used herein, embraces administration of these agents in a sequential manner, that is, wherein each therapeutic agent is administered at a different time, as well as administration of these therapeutic agents, or at least two of the agents, in a substantially simultaneous manner.

Sequential or substantially simultaneous administration of each agent can be affected by any appropriate route including, but not limited to, oral routes, intravenous routes, intramuscular, subcutaneous routes, and direct absorption through mucous membrane tissues. The agents can be administered by the same route or by different routes. For example, a first agent can be administered orally, and a second agent can be administered intravenously.

As used herein, the term “sequential” means, unless otherwise specified, characterized by a regular sequence or order, e.g., if a dosage regimen includes the administration of a first therapeutic agent and a second therapeutic agent, a sequential dosage regimen could include administration of the first therapeutic agent before, simultaneously, substantially simultaneously, or after administration of the second therapeutic agent, but both agents will be administered in a regular sequence or order. The term “separate” means, unless otherwise specified, to keep apart one from the other. The term “simultaneously” means, unless otherwise specified, happening or done at the same time, e.g., the agents of the invention are administered at the same time. The term “substantially simultaneously” means that the agents are administered within minutes of each other (e.g., within 10 minutes of each other) and intends to embrace joint administration as well as consecutive administration, but if the administration is consecutive it is separated in time for only a short period (e.g., the time it would take a medical practitioner to administer two agents separately). As used herein, concurrent administration and substantially simultaneous administration are used interchangeably. Sequential administration refers to temporally separated administration of the agents described herein.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

In order that the invention described herein may be more fully understood, the following examples are set forth. The examples described in this application are offered to illustrate the methods, compositions, and systems provided herein and are not to be construed in any way as limiting their scope.

### Materials and Methods

### Endometrial Sample Collection

The Clinical Research Ethics Committee (Comites de Etica en Investigacion Clinica) of Hospital La Fe (Valencia, Spain) approved the endometrial sample collection described herein and written informed consent was obtained from all donors prior to tissue collection. Samples were collected from women who were pregnant 1 to 5 years prior to the study. All donors had regular menses, with no underlying endometrial pathology and had not received hormonal therapy in the 3 months preceding sample collection. Endometrial biopsies were obtained by pipelle (Genetics, Belgium) under sterile conditions in the early luteal phase (cycle day 15-17). Samples were kept in PBS prior to processing within 6 hours. Clinical features of the previously sPE and normal pregnancies are summarized in Tables 2 and 3.

TABLE 2
Maternal and neonatal characteristics of endometrial
donors (in vitro decidualization analyses).

| | Normal Pregnancy | | sPE* | | |
|---|---|---|---|---|---|
| | (n = 13) | | (n = 13) | | P** |
| Maternal Age (years) | 37.8 | (0.8) | 35.2 | (1.4) | >0.05 |
| Systolic blood pressure | 121.6 | (2.5) | 161.5 | (2.7) | <0.001 |
| (mmHg) | | | | | |
| Diastolic blood pressure | 74.1 | (1.7) | 100.9 | (2.9) | <0.001 |
| (mmHg) | | | | | |
| Proteinuria | 0 or NA | | +1 to +2 | | <0.05 |
| Gestational age at | 39.5 | (0.3) | 33.7 | (1.0) | <0.001 |
| delivery (weeks) | | | | | |
| Birth weight (g) | 3250 | (111.4) | 1844 | (190.9) | <0.001 |
| Parity (n) | 1.5 | (0.2) | 1.8 | (0.2) | >0.05 |
| Interval from pregnancy | 3.9 | (1.2) | 2.2 | (0.3) | >0.05 |
| to endometrial biopsy | | | | | |
| (years) | | | | | |

mean ± SEM
**One-tailed Student's t-test
NA: Not Available

TABLE 3
Maternal and neonatal characteristics of
endometrial donors (transcriptomic analyses
of decidual gene expression in vitro).

| | Normal Pregnancy | | sPE* | | |
|---|---|---|---|---|---|
| | (n = 7) | | (n = 5) | | P** |
| Maternal Age (years) | 36.6 | (1.3) | 32.2 | (3.0) | >0.05 |
| Systolic blood pressure | 126.0 | (2.2) | 161.6 | (3.7) | <0.001 |
| (mmHg) | | | | | |
| Diastolic blood pressure | 74.3 | (1.9) | 109.4 | (5.1) | <0.001 |
| (mmHg) | | | | | |
| Proteinuria | 0 or NA | | +1 to +2 | | <0.01 |
| Gestational age at | 39.3 | (0.3) | 37.1 | (0.4) | <0.01 |
| delivery (weeks) | | | | | |
| Birth weight (g) | 3110.1 | (177.3) | 2611 | (200.7) | <0.05 |
| Parity (n) | 1.6 | (0.2) | 1.6 | (0.4) | >0.05 |
| Interval from pregnancy | 2.3 | (0.5) | 3.1 | (0.8) | >0.05 |
| to endometrial biopsy | | | | | |
| (years) | | | | | |

*sPE included 1 cases of Hemolysis Elevated Liver Low Platelet (HELLP) syndrome and 1 case of eclampsia.
**One-tailed Student's t-test
mean ± SEM
NA: Not Available

hESC Isolation and Culture

Endometrial samples were processed and hESCs were isolated by mild collagenase digestion and cultured as previously described (Simon et al., J Clin Endocinol Metab, 1994, 78(3):675-682).

### In Vitro Decidualization

hESCs were decidualized via cAMP and MPA treatment as previously described (Brar et al., Endocrine, 1997, 6(3):301-307). hESCs were cultured in parallel without additives as controls.

### F-Actin Staining

Confirmation of decidualization at the morphological level was accomplished by F-actin staining as previously described (Garrido-Gomez et al, FASEB J, 2012, 26(9):3715-3727).

### PRL and IGFBP1 ELISA

Conditioned medium from cultured hESCs was collected at day 5 of decidualization. PRL (Boster Immunoleader, USA) and IGFBP1 (Raybiotech, USA) concentrations were assayed by using commercial ELISA kits according to the manufacturers' instructions.

Transcriptome-Wide Analyses of hESC Decidualized In Vitro

Decidualized and non-decidualized hESCs from donors who had sPE (n=5) or normal pregnancies (n=7) were collected after 5 days in culture and total RNA was extracted into Trizol according to the manufacturer's instructions (Life Technologies). The RNA quality was assessed using an RNA LabChip and an A2100 Bioanalyzer (Agilent Technologies). Samples with an RNA integrity >7 were selected for microarray analyses. Sample preparation and hybridization was accomplished using Agilent 2100 Bioanalyzer technology according to the manufacturer's guidelines. Hybridized microarrays were imaged with an Axon 4100A scanner (Molecular Devices) and the data were extracted with the GenePix Pro 6.0 software (Molecular Devices). Gene expression values were preprocessed (half-background median intensity values were subtracted from the average intensity of each spot), normalized (Bioconductor LIMMA package in the R software) and statistically analyzed by ANOVA. The differentially expressed genes (p-value<0.05) were clustered by using UPGMA and the Pearson correlation option. Fold changes were estimated by LIMMA. p value corrections were performed by using the false discovery rate (Benjamini Y et al., Behav Brain Res, 2001, 125(1-2):279-284) to account for multiple testing effects. The data were deposited in the Gene Expression Omnibus (accession number GSE94644).

qRT-PCR Validation of In Vitro Gene Expression Data

Relative expression levels of four genes (ALDH1A1, IGFBP1, NANOS3, and HSD17B2) were determined by qRT-PCR, using (3-actin as an internal control. Specific primers for each gene are provided in Table 4.

TABLE 4
Primer pairs used for expression level determination by qRT-PCR.

| Gene | Forward Primer | SEQ ID NO.: | Reverse Primer | SEQ ID NO.: |
|---|---|---|---|---|
| ALDH1A1 | 5′-agatgacgtgatcaaaagagca | SEQ ID NO.: 1 | 5′-cagacatcttgaatccaccaaa | SEQ ID NO.: 2 |
| IGFBP1 | 5′-atggcatacctcaacgccaa | SEQ ID NO.: 3 | 5′-aaggacttgctcgttggaca | SEQ ID NO.: 4 |
| NANOS3 | 5′-gggaaagagggtcctgaaac | SEQ ID NO.: 5 | 5′-agcacgtgggactggtagat | SEQ ID NO.: 6 |
| HSD17B2 | 5′-agtctgcctgctcatcctgt | SEQ ID NO.: 7 | 5′-ttatctgcatcggcttcgtg | SEQ ID NO.: 8 |
| β-actin | 5′-cacactgtgcccatctacga | SEQ ID NO.: 9 | 5′-tagctcttctccagggagga | SEQ ID NO.: 10 |

### Collection of Placentas and Fetal Membranes

The UCSF Institutional Review Board approved the collection of placentas and fetal membranes described herein. Written informed consent was obtained from all donors. Specimens were collected immediately after delivery. Samples were obtained within 1 h of delivery, washed in PBS, transferred to ‘Cytowash medium’ (DMEM H-21, 1% glutamine plus, 1% penicillin/streptomycin, 0.1% gentamycin) supplemented with 2.5% FBS, and placed on ice prior to processing. The clinical characteristics of the severe preeclampsia (sPE) and gestational age-matched noninfected preterm birth (nPTB) pregnancies are summarized in Tables 5 and 6.

TABLE 5
Maternal and neonatal characteristics of decidua donors (transcriptomic
analyses of decidual gene expression in situ).

| | nPTB | | sPE | | |
|---|---|---|---|---|---|
| | (n = 4) | | (n = 4) | | P** |
| Maternal Age (years) | 31.7 | (2.3) | 29.0 | (3.3) | >0.05 |
| Systolic blood pressure | 117.8 | (7.8) | 152.0 | (6.9) | <0.01 |
| (mmHg) | | | | | |
| Diastolic blood pressure | 72.7 | (5.0) | 91.6 | (3.3) | <0.01 |
| (mmHg) | | | | | |
| Proteinuria | 0 or NA | | +1 to +3 | | <0.05 |
| Gestational age at delivery | 30.2 | (2.6) | 28.8 | (1.7) | >0.05 |
| (weeks) | | | | | |
| Birth weight (g) | 2083.3 | (207.9) | 908.7 | (177.8) | <0.01 |

mean ± SEM
**One-tailed Student's t-test
NA: Not Available

TABLE 6
Maternal and neonatal characteristics of
decidual donors (immunolocalization and
in vitro differentiation experiments).

| | nPTB | | sPE | | |
|---|---|---|---|---|---|
| | (n = 5) | | (n = 7) | | P** |
| Maternal Age (years) | 30.8 | (1.7) | 27.9 | (2.9) | >0.05 |
| Systolic blood pressure | 116.5 | (5.3) | 150.5 | (6.6) | <0.01 |
| (mmHg) | | | | | |
| Diastolic blood pressure | 68.2 | (4.6) | 88.0 | (3.6) | <0.01 |
| (mmHg) | | | | | |
| Proteinuria | 0 or NA | | +1 to +2 | | <0.05 |
| Gestational age at delivery | 39.3 | (0.3) | 37.1 | (0.4) | >0.05 |
| (weeks) | | | | | |
| Birth weight (g) | 3110.1 | (177.3) | 2611 | (200.7) | <0.05 |

mean ± SEM
**One-tailed Student's t-test
NA: Not Available

### Laser Microdissection and Microarray Analyses

Laser microdissection was used to isolate the decidua basalis and parietalis from sPE and nPTB samples (n=4/group). Biopsies of the placenta/decidua basalis and smooth chorion/decidua parietalis were washed repeatedly in cold PBS to remove blood contaminants. Areas that showed injury and/or necrosis of the tissue were discarded. Samples were then placed in cryomolds containing OCT, frozen over a dry ice/ethanol slurry, and stored at −80° C. The blocks were sectioned at 20 μm using a Leica CM3050 cryostat. The sections were mounted on UV treated PEN-membrane slides, and stored under ice prior to laser microdissection later that day. Immediately prior to the procedure, sections were immersed in cold PBS until the OCT dissolved (-1 min), dipped in 0.1% toluidine blue for 30 seconds, washed in cold PBS, dehydrated (30 s/treatment) in a graded ethanol series (75%, 75%, 95%, 100%), then rapidly dried with compressed nitrogen. All solutions were made with nuclease-free water. Decidua basalis and parietalis were laser microdissected (Leica LMD 7000) and collected directly into RLT Plus (Qiagen RNeasy Plus 518 Micro kit). Total RNA was isolated according to the manufacturer's protocol and concentrations were measured photometrically (NanoDrop 2000c). RNA integrity was determined via microfluidic phoresis (Agilent Bioanalyzer 2100). The samples were stored at -80° C.

Global gene profiling was accomplished by using the GeneChipHuGene 2.0 ST array (Affymetrix). Sample processing and hybridization were done according to protocols that were devised by the UCSF Gladstone (NHLBI) Genomics Core Facility. Gene level expression data quality was confirmed, normalized (RMA) and summarized (Affymetrix Expression Console Software). Significant differential expression was determined by statistical analysis of false discovery rate (FDR <0.05) and absolute linear fold change >2 (Transcriptome Analysis Console Software).

### Immunofluorescence of Tissue Sections

Samples were fixed in paraformaldehyde, frozen in OCT and immunostained as previously described (Genbacev et al., Hum Reprod, 2016, 31(6):1300-1314). The sources and concentrations of antibodies used in the study described here are listed in Table 7.

TABLE 7
Antibodies used for the immunocyto
and histochemistry experiments.

| | Catalog | | |
|---|---|---|---|
| Antibody | Number/Clone | Source | Dilution |
| PRL | PA5-26006 | Thermo Fisher | 1:50 |
| IGFBP1 | ab111203 | Abcam | 1:50 |
| Vimentin | V4630 | Sigma-Aldrich | 1:100 |
| CK7 | 7D3 | Damsky et al., 1992 | 1:100 |
| PEG1/MEST | LS-C346142 | LifeSpan Biosciencies | 1:50 |
| BMP2 | ab14933 | Abcam | 1:50 |
| PRG2 | NBP1-88573 | Novus Bio | 1:100 |
| anti-rabbit secondary | A21206 | Life Technologies | 1:1000 |
| Ab | | | |
| anti-goat secondary | A11055 | Life Technologies | 1:1000 |
| Ab | | | |
| anti-mouse secondary | A21907 | Life Technologies | 1:1000 |
| Ab | | | |
| anti-rat secondary | 712-025-153 | Jackson Immuno | 1:100 |
| Ab | | | |

Stromal Cell Isolation from Decidua Basalis and Parietalis

Samples from the basal plate and smooth chorion/decidua parietalis were washed with cold sterile PBS (Ca++-Mg++-free) supplemented with 1% penicillin-streptomycin, 0.25% fungizone, and 0.1% gentamicin. A thin layer of the decidua basalis (0.5-2.0 mm) was cut from the basal plate and dissected into small pieces (3-4 mm²), which were washed again in PBS containing antibiotics and antimycotics. To isolate the decidua parietalis, the amnion was manually separated from the smooth chorion. Then the decidual layer was gently scraped from the maternal surface of the chorionic CTBs and washed again in PBS containing antibiotics and antimycotics. Small pieces of the decidua basalis and parietalis were subjected to a series of enzymatic digestion steps. The first collagenase digestion (15-20 min) was in 1× PBS (10 ml/g of tissue) containing 35 mg collagenase type I (Sigma, USA), 40 mg DNase (Sigma, USA), 69 mg hyaluronidase (Sigma, USA), and 100 mg BSA (Sigma, USA) per 100 ml. The supernatant was discarded. Then the tissue was incubated (second digestion) for 25-30 min in 1× PBS containing 6.9 mg trypsin (Sigma, USA), 20 mg EDTA (Invitrogen, USA) and 40 mg DNase (Sigma, USA) per 100 ml. The digestion was carried out at 37° C. with gentle shaking in a water bath at a ratio of tissue (g) to dissociation buffer volume (ml) of 1:9. Enzyme activity was stopped by adding an equal volume of Cytowash medium containing 10% FBS. The supernatant (cell suspension) was filtered through a 70 p.m sterile strainer and centrifuged at 1,200 ×g for 7 min. An additional collagenase treatment (third digestion) was performed by adding 7× collagenase digestion buffer (see above) calculated on the basis of the weight of the cell pellet (g), followed by incubation for 15-20 min at 37° C. with gentle shaking in a water bath. The supernatant (cell suspension) was collected a second time by centrifugation. The cell pellets from the trypsin and second collagenase digestion were combined and purified over a Percoll (Sigma, USA) gradient (44). The gradient was centrifuged at 2,700 ×g for 25 min (4° C.) and the 20-40% density fraction was collected. After repeatedly washing with Cytowash medium, the isolated decidual cells were grown in DMEM F12 containing 10% charcoal-stripped FBS and 0.1% penicillin-streptomycin.

### Immunofluorescence of Cultured Cells

Glass coverslips were incubated with 50 μL, of 0.5% gelatin (Sigma, USA) for 30 min at 37° C. Cells isolated from the decidua basalis or decidua parietalis were cultured on the coated glass coverslips until cells reached confluence. Then cells were immunostained as previously described (Genbacev et al., Hum Reprod, 2016, 31(6):1300-1314).

Re-Decidualization In vitro

Stromal cells isolated from the decidua basalis or decidua parietalis were passaged (p) five times. The cells rapidly reverted to a non-decidualized morphological phenotype (p 1-2). At p3 or p4, the cells were decidualized and analyzed (morphology, immunolocalization and ELISA) as described herein.

### CTB Invasion Assay

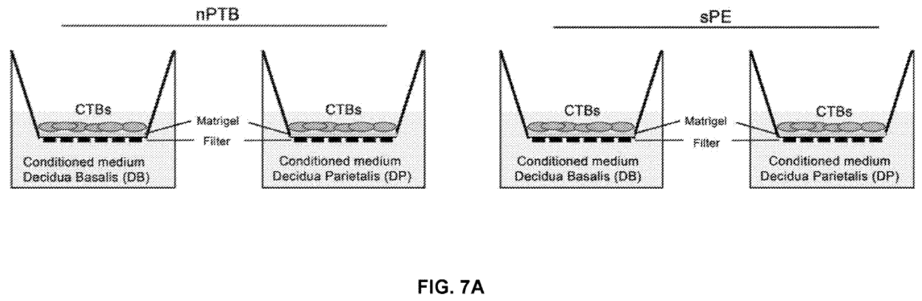

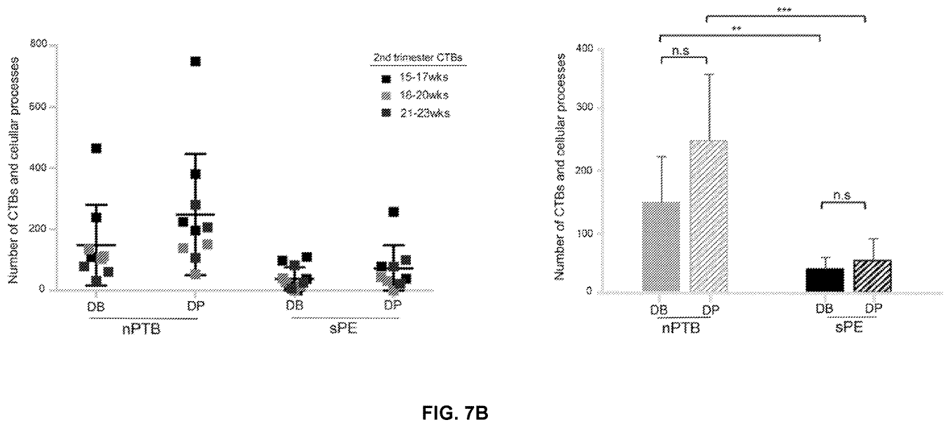

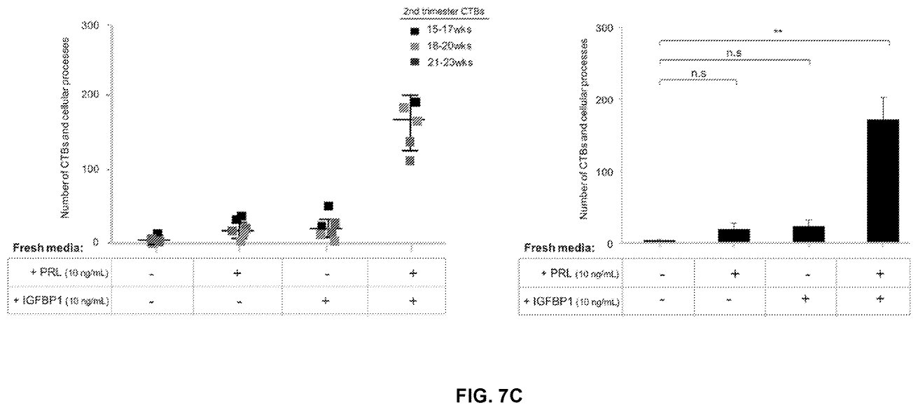

CTBs were isolated from second trimester human placentas as previously described (Kliman et al., Endocrinology, 1986, 118(4):1567-1582; Hunkapiller et al., Development, 2011, 138(14):2987-2998). Invasion was quantified by using Transwell polycarbonate inserts (6.5 mm) with 8-μm pores that were coated with 10 ᵢll of undiluted Matrigel (Corning Corp, USA). Briefly, CTBs (isolated from 10 placentas) were plated at a density of 250,000 cells per insert in 24-well plates with 400 μL of conditioned medium from freshly isolated cells of the decidua basalis or decidua parietalis that were cultured overnight (p0; see FIG. 7A). PRL (10 ng/ml; Boster Immunoleader, USA) and/or IGFBP1 (10 ng/ml; Raybiotech, USA) was added to fresh medium and the effects on CTB invasion were quantified as compared to the same medium with no additives. The experimental and control conditions were tested in duplicate. Invasion was assayed as previously described (Genbacev et al., Hum Reprod, 2016, 31(6):1300-1314). The entire experiment was repeated 4 times. The average value of the duplicate measurements was calculated. The results were plotted as the mean±SEM. Student's t-test was used to analyze the differences among the groups.

### Statistics

Data were shown as the mean values±SEM and n denoted the number of experiments. Students' t-distribution was used to analyze global differences between groups. A p-value of ≤0.05 was considered significant.

### Example 1

### Failure of Human Endometrial Stromal Cells from Women with a Prior sPE Pregnancy to Decidualize In Vitro

Decidualization of hESCs isolated from endometrial biopsies of patients who developed sPE in a previous pregnancy (n=13) were assessed and compared to control patients who had normal obstetric outcomes (n=13). The maternal and neonatal characteristics of the participants are summarized in Table 2. hESCs were decidualized by treatment with cAMP and medroxyprogesterone acetate (MPA) for 5 days. As experimental controls, cells from the same donor were cultured in parallel in the absence of cAMP and MPA.

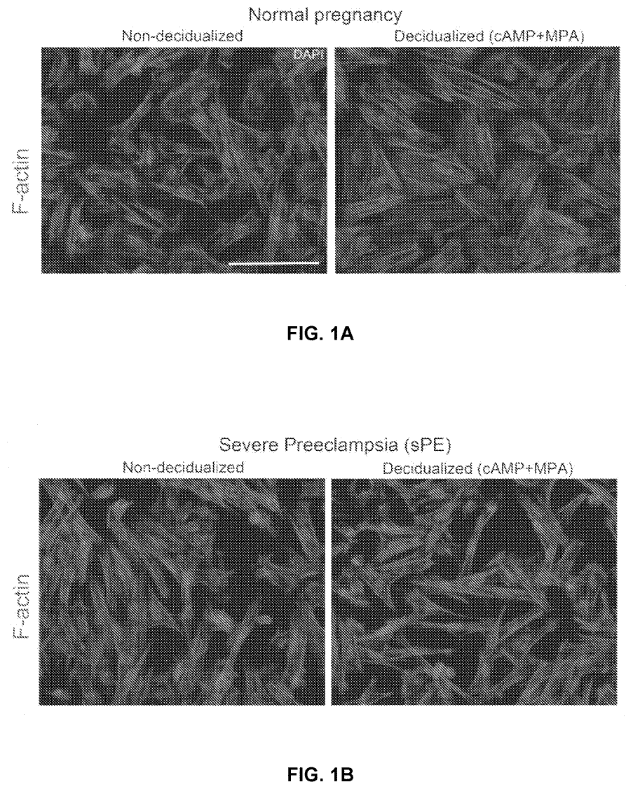

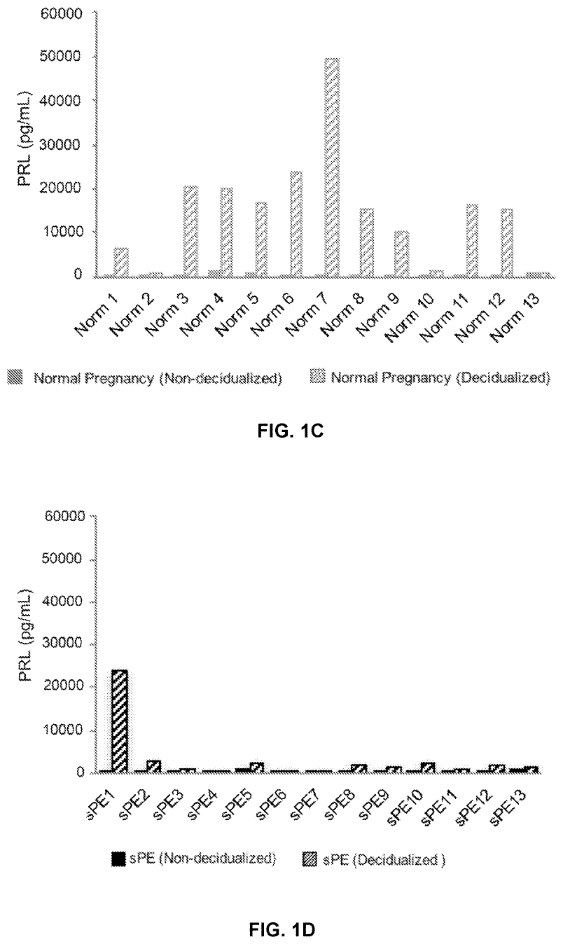

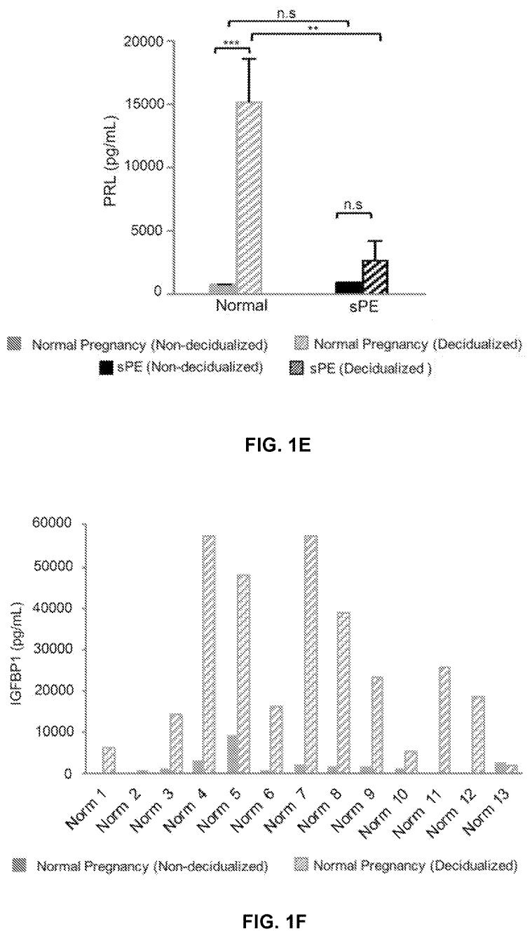

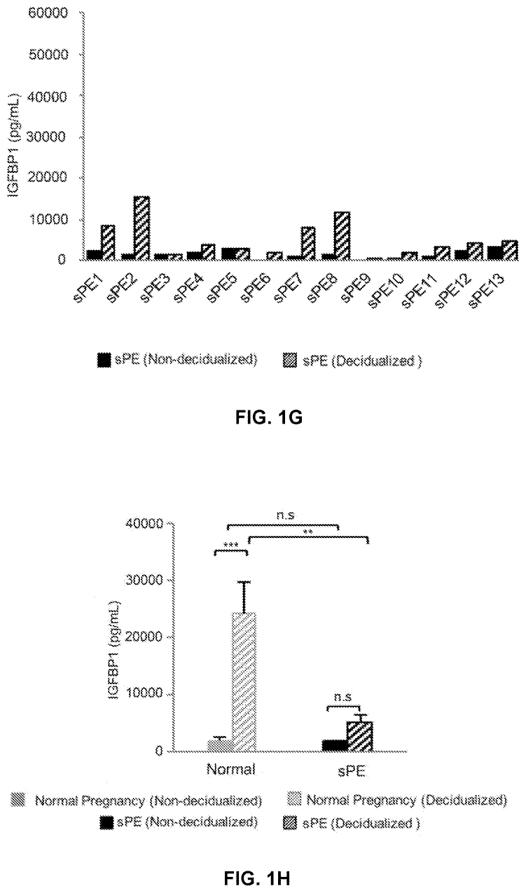

Localization of F-actin in decidualized cells from women with uncomplicated pregnancies showed the expected cytoskeletal reorganization and shape changes that were consistent with transformation from a fibroblast to a decidual phenotype (FIG. 1A). In contrast, hESCs from women who had sPE failed to undergo these changes (FIG. 1B). In non-decidualized hESCs, PRL (FIGS. 1C-1E) and IGFBP1 (FIGS. 1F-1H) levels detected in conditioned medium were low and not statistically different between the two groups. Secretion of both molecules greatly increased upon decidualization of most of the control cultures, but hESCs from former sPE patients failed to show an increase (FIGS. 1D, 1E, 1G and 1H).

Thus, the results suggested that in vitro decidualization was impaired in hESCs obtained from former sPE patients as compared to controls.

### Example 2

### Alterations in the Global Transcriptional Profiles of Decidualized hESCs from Former sPE Patients

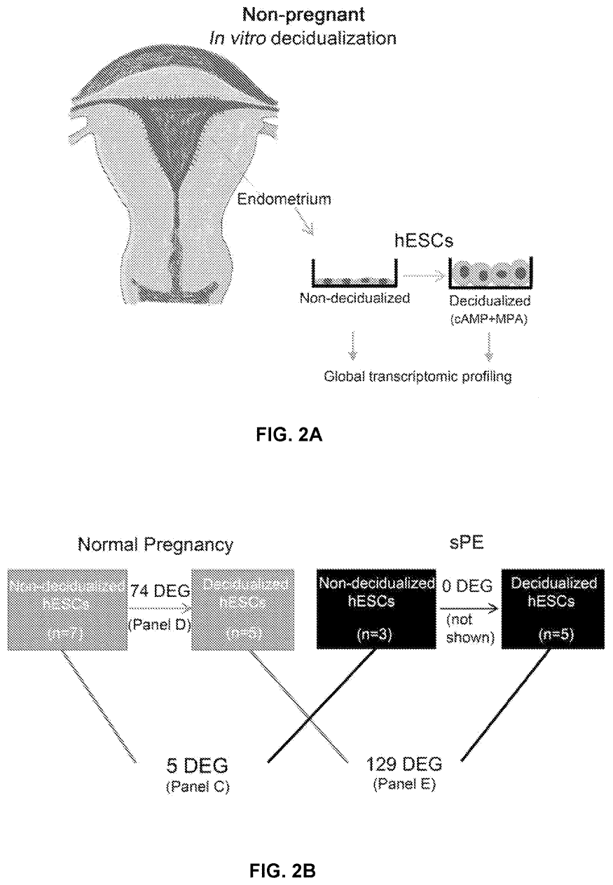

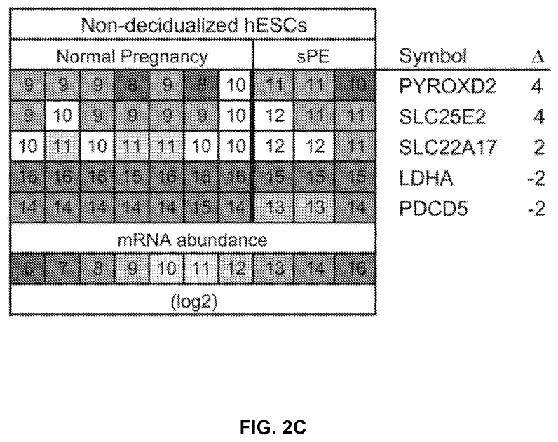

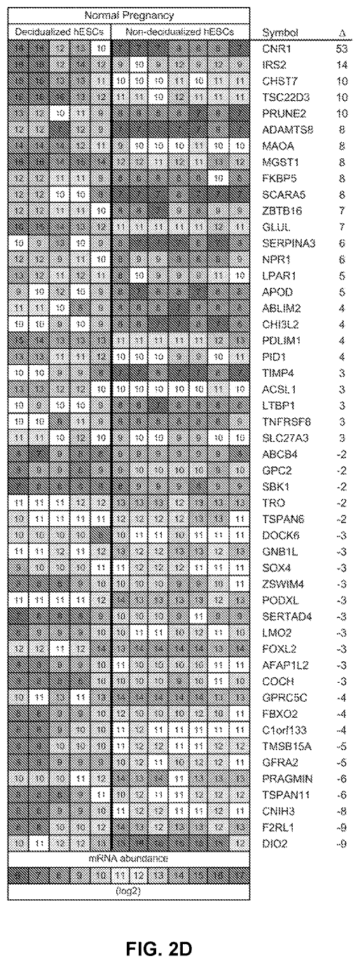

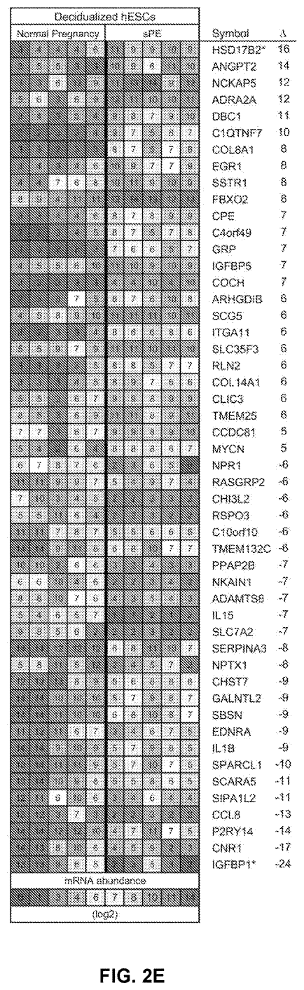

To identify the molecular changes underlying the functional decidualization defect found in hESCs from women who had experienced sPE, a microarray strategy was used. Specifically, a transcriptomic analysis of non-decidualized and decidualized hESCs established from normal pregnancy and sPE pregnancy groups were carried out in vitro (FIG. 2A). The clinical characteristics of the endometrial donors are shown in Table 3.

An overview of the results is presented in FIG. 2B. In the non-decidualized state, only 5 genes were differentially expressed between the control and the sPE samples, and the fold-differences were modest (FIG. 2C). Thus, in a basal state, the hESCs from former sPE patients were very similar to those from control women.

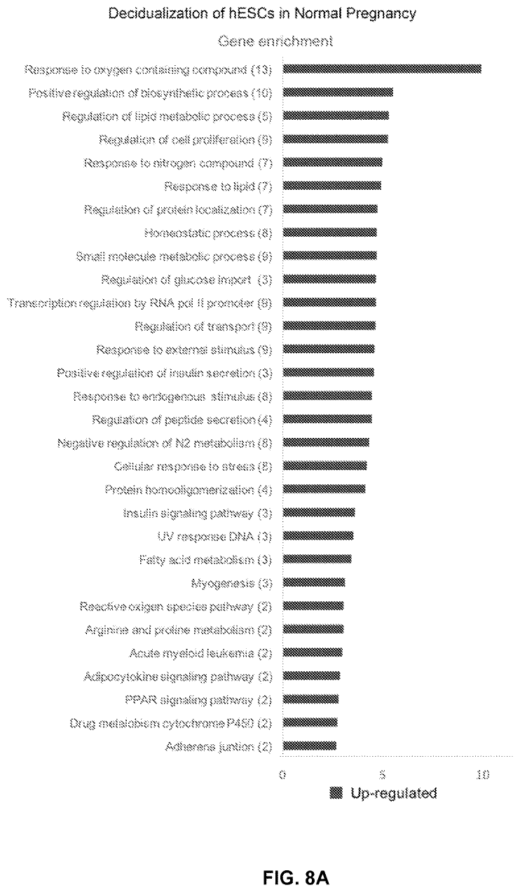

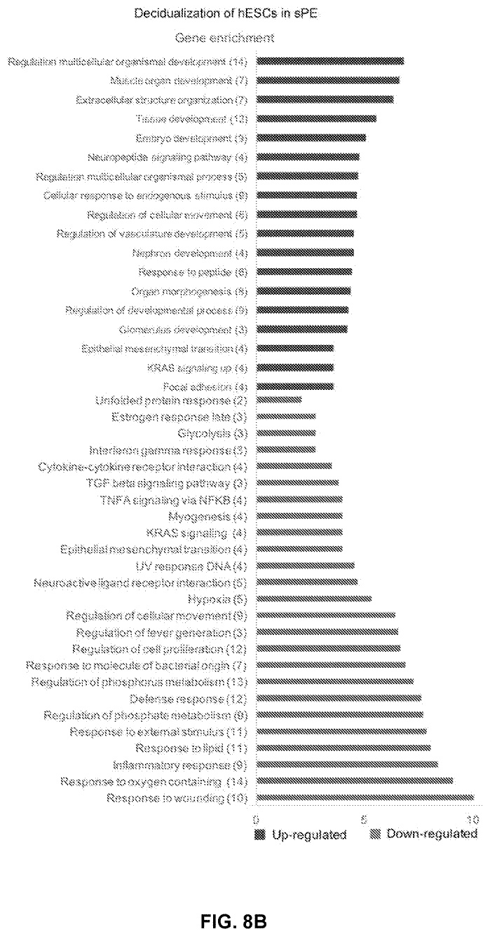

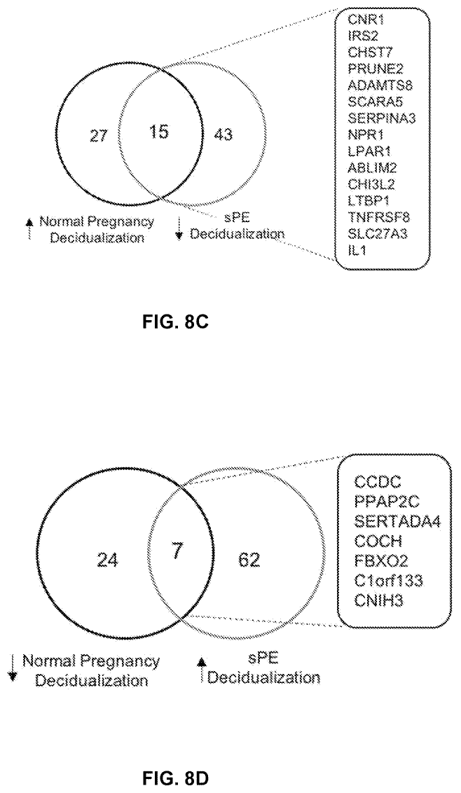

During decidualization of the samples from control donors, the expression of 74 genes was significantly regulated by ≥2-fold (FIG. 2D and FIG. 13). The results included the up-regulation of genes (e.g., CNR1, IRS2 and MAOA) and down-regulation of genes (e.g., COCH, SERTADA4 and CNIH3) that are well known to be involved in decidualization. At the pathway level, processes that are relevant to decidualization—including regulation of oxygen responses, insulin secretion and proliferation—were up regulated (FIG. 8A). No significantly down regulated pathways were detected.

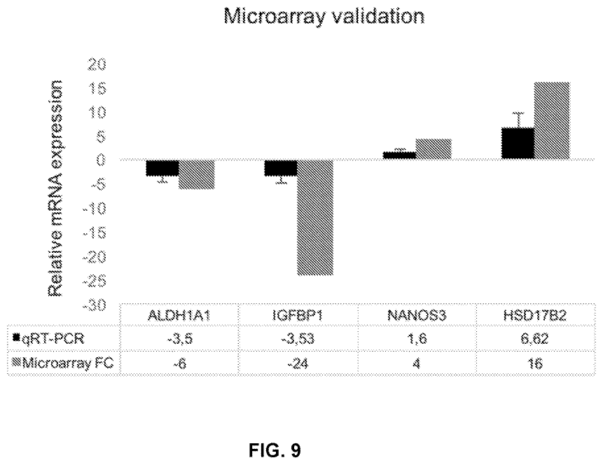

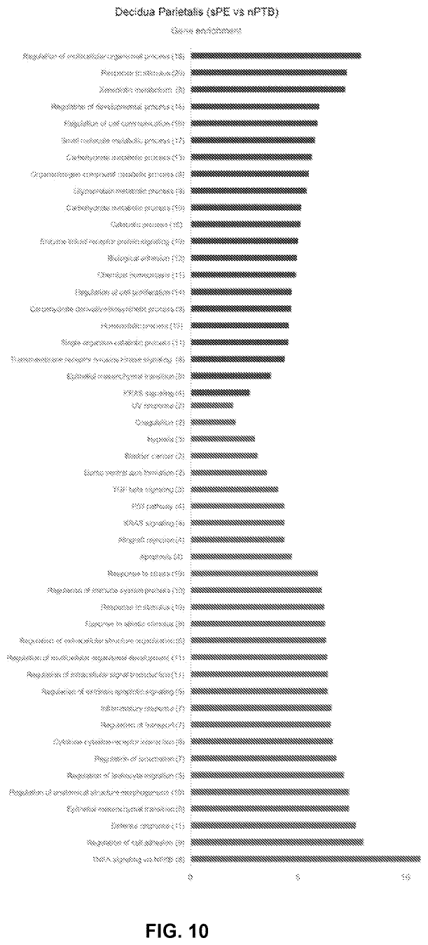

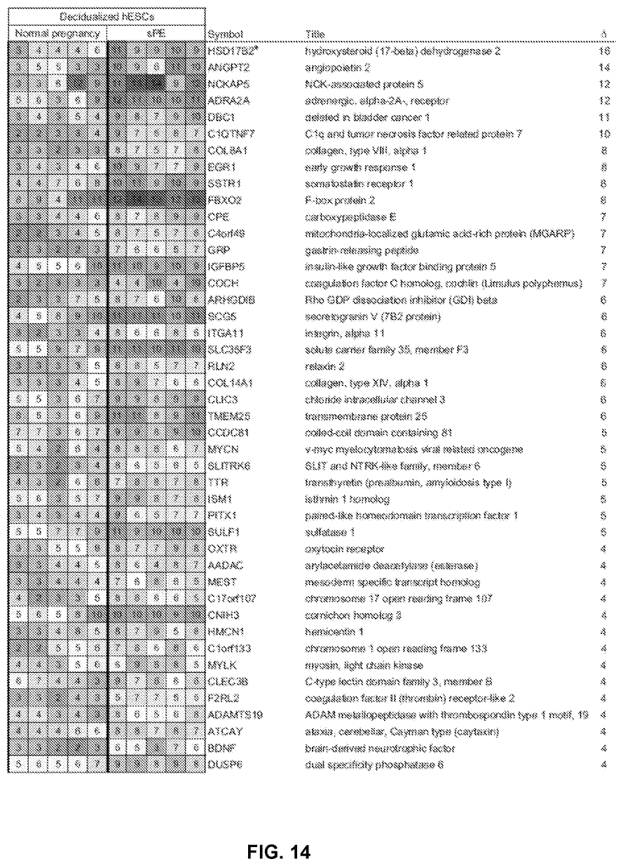

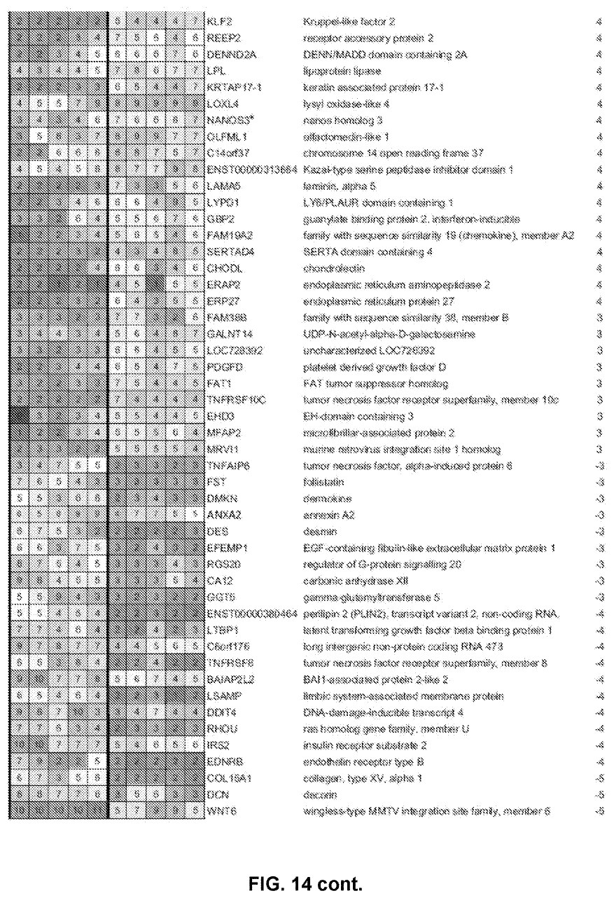

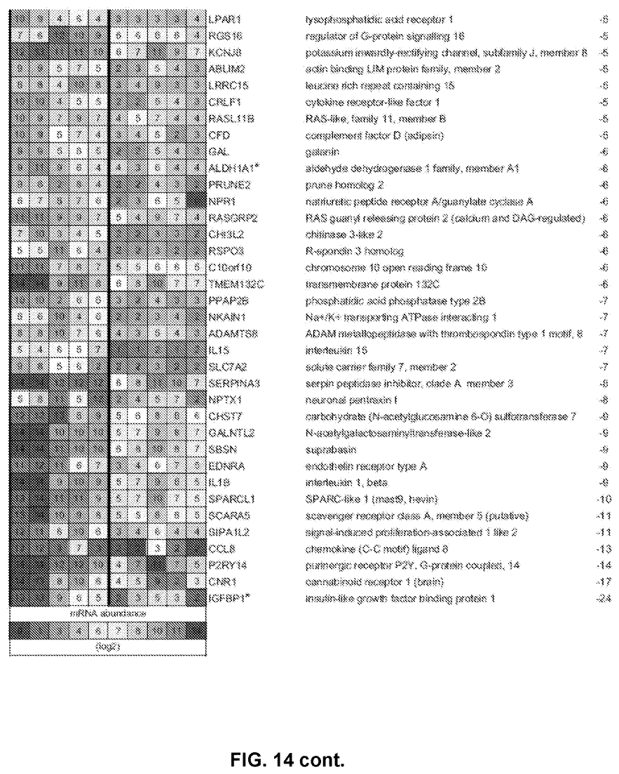

Consistent with the results shown in FIGS. 1A-1H, the comparison between non-decidualized and decidualized hESCs isolated from former sPE patients failed to detect modulated gene expression (0 DEGs; FIG. 2B). In contrast, comparing the transcriptomes of the decidualized cells in the two groups revealed 129 misexpressed genes (≥2-fold; FIG. 2E and FIG. 14). mRNAs whose expression patterns were validated by qRT-PCR analyses (FIG. 9) are denoted with an asterisk in FIG. 2E and FIG. 14. The DE genes included the up regulation of mRNAs encoding molecules that are involved in hormone conversion (HSD17B2), extracellular structure organization (LAMAS, SULF1 and ITGA11), vascular development (ANGPT2, EGR1 and RELAXIN2) and response to peptides (KLF2, SSTR1 and IGBFP5) (FIG. 8B). The down regulated category included genes that play important roles in decidualization (e.g., IGFBP1, CNR1 and IL-1B). The latter group functioned in numerous pathways such as cytokine-receptor interactions, wounding response, inflammation response, estrogen response, and TGF-beta signaling (FIG. 8B).

Finally, the overlap between the DE genes in the non-decidualized vs. decidualized hESCs from women who had normal pregnancies (FIG. 2D) and the sPE vs. normal pregnancy group following decidualization (FIG. 2E) was analyzed. Fifteen genes were up regulated during normal hESC decidualization and down regulated during decidualization of hESC from sPE women. They included signaling molecules such as CNR1, IRS2, LPAR1, ABLIM2 and LTBP1, all of which have important functions during decidualization (FIG. 8C). In contrast, 7 genes were downregulated during normal decidualization and upregulated in the equivalent samples from former sPE patients (FIG. 8D). They included molecules such as LOCH and CNIH3.

Thus, global transcriptional profiling of decidua basalis and the decidua parietalis as described herein revealed differentially expressed genes in sPE pregnancies compared to control pregnancies.

### Example 3

### Molecular Defects In Situ of Decidua Basalis or Decidua Parietalis from Control vs. sPE Pregnancies

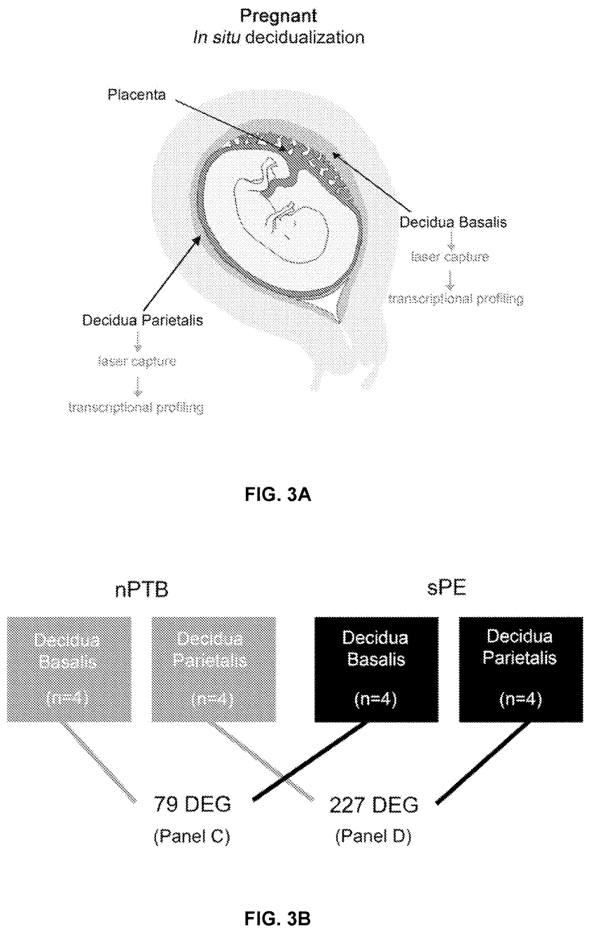

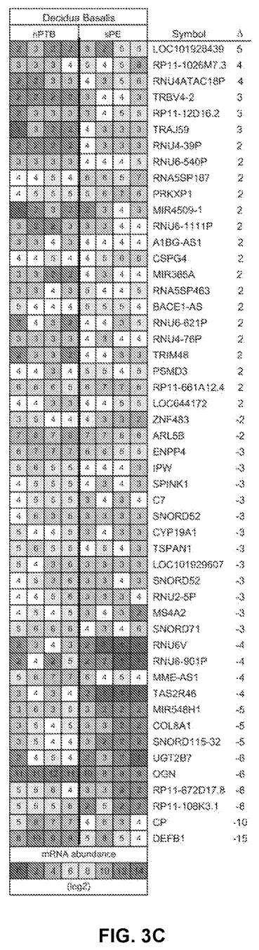

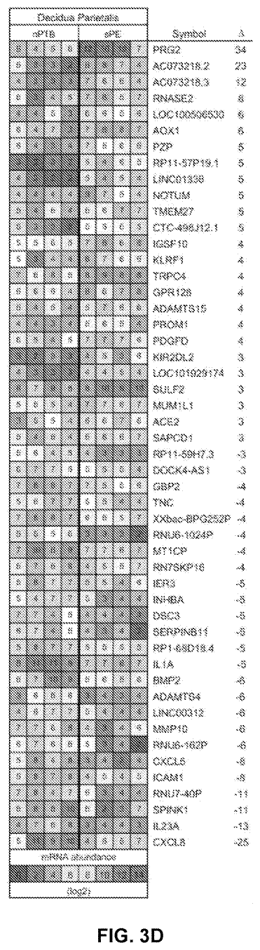

A laser microdis section approach was used to isolate portions of the decidua basalis (DB) or decidua parietalis (DP). Cells were captured from tissue sections of biopsy specimens from cases of women with sPE vs. controls (gestational age-matched samples from women who had a preterm birth with no signs of infection nPTB; FIG. 3A). The clinical characteristics of the participants are summarized in Table 8.

TABLE 8
Maternal and neonatal characteristics of decidua donors (transcriptomic
analyses of decidual gene expression in situ of severe preeclampsia
(sPE) vs. spontaneous preterm birth with no signs of infection
(noninfected preterm birth; nPTB).

| | nPTB | | sPE | | |
|---|---|---|---|---|---|
| | (n = 4) | | (n = 4) | | P** |
| Maternal Age (years) | 31.7 | (2.3) | 29.0 | (3.3) | >0.05 |
| Systolic blood pressure | 117.8 | (7.8) | 152.0 | (6.9) | <0.01 |
| (mmHg) | | | | | |
| Diastolic blood pressure | 72.7 | (5.0) | 91.6 | (3.3) | <0.01 |
| (mmHg) | | | | | |
| Proteinuria | 0 or NA | | +1 to +3 | | <0.05 |
| Gestational age at delivery | 30.2 | (2.6) | 28.8 | (1.7) | >0.05 |
| (weeks) | | | | | |
| Birth weight (g) | 2083.3 | (207.9) | 908.7 | (177.8) | <0.01 |

mean ± SEM
**One-tailed Student's t-test
NA: Not Available

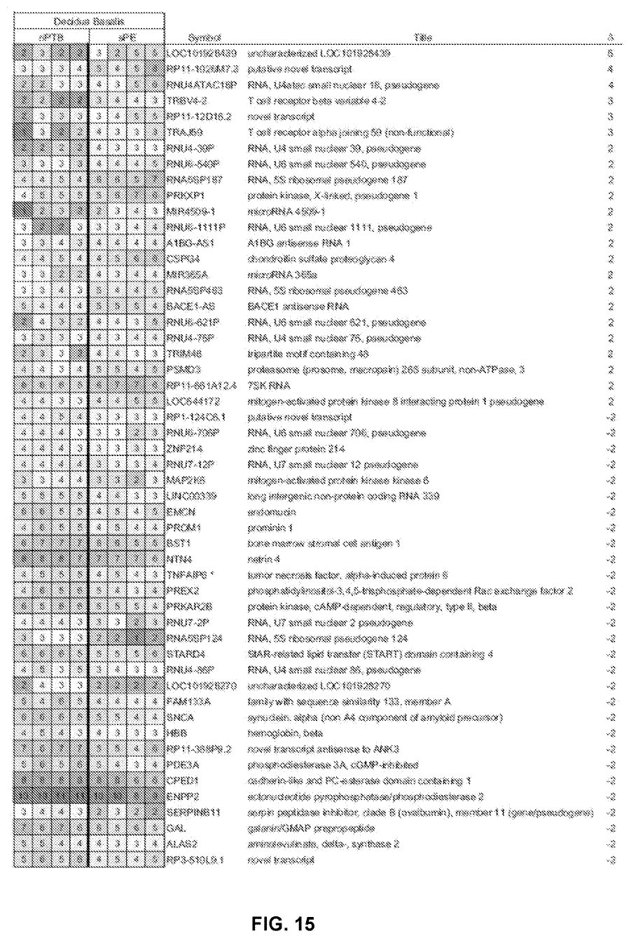

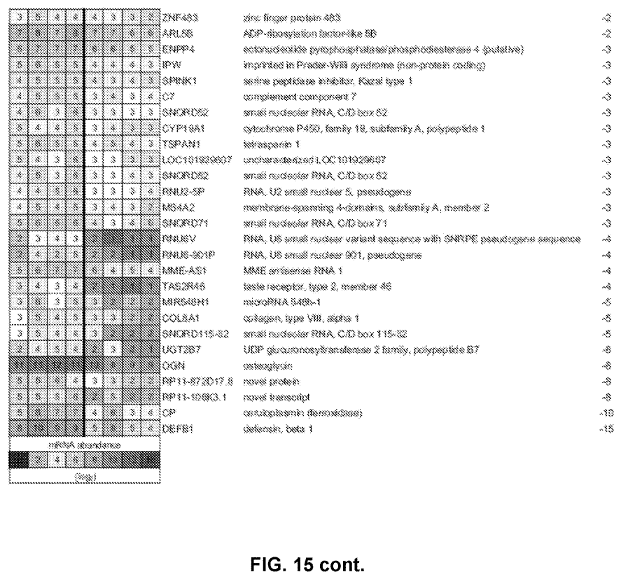

An overview of the results is shown in FIG. 3B. In the decidua basalis, 79 genes were significantly DE in sPE vs. nPTB with modest fold changes (FIG. 3C and FIG. 15). The genes included the upregulation of mRNAs encoding molecules involved in RNA processing. Downregulated genes included DEFB1, CP, OGN and COL8A1.

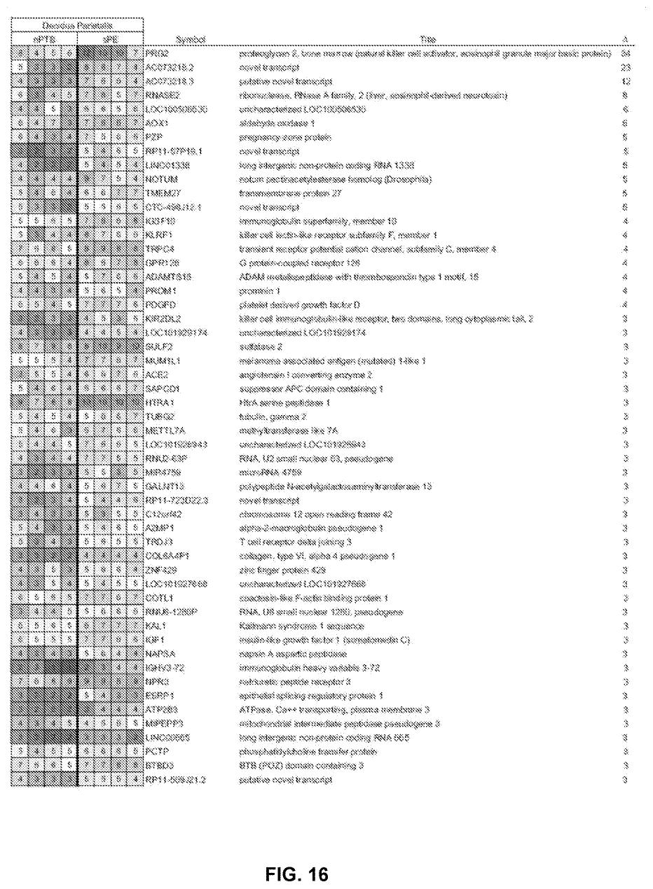

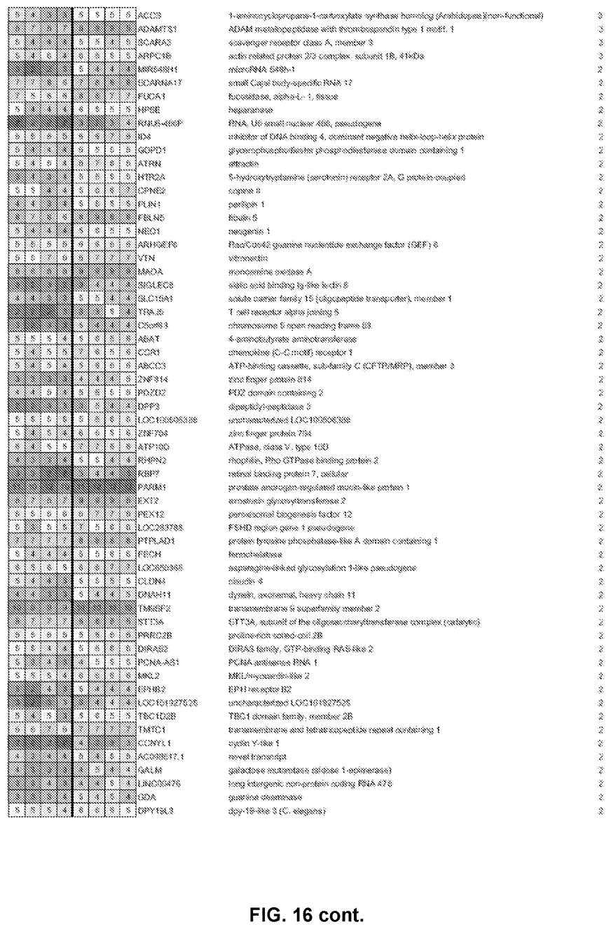

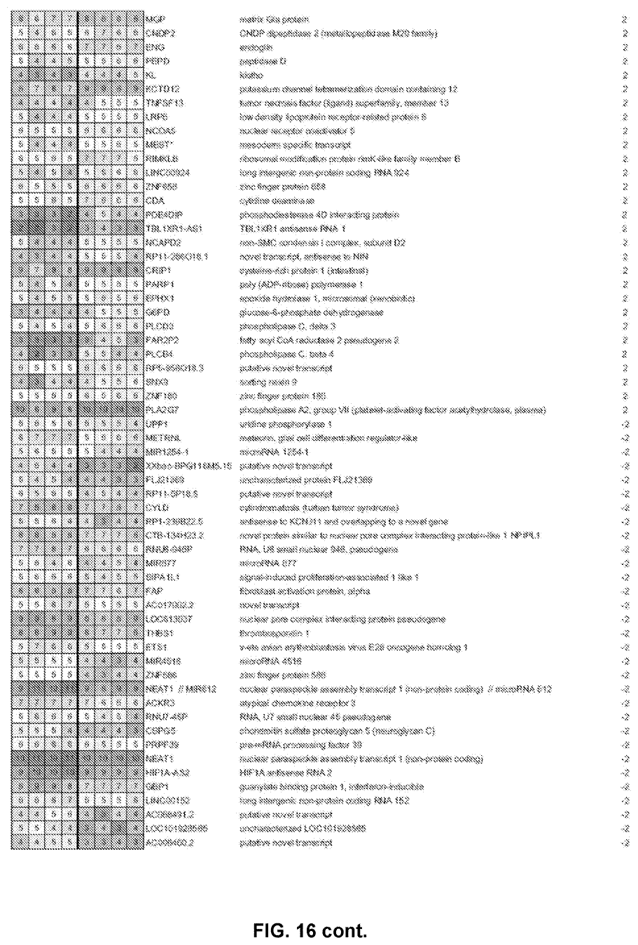

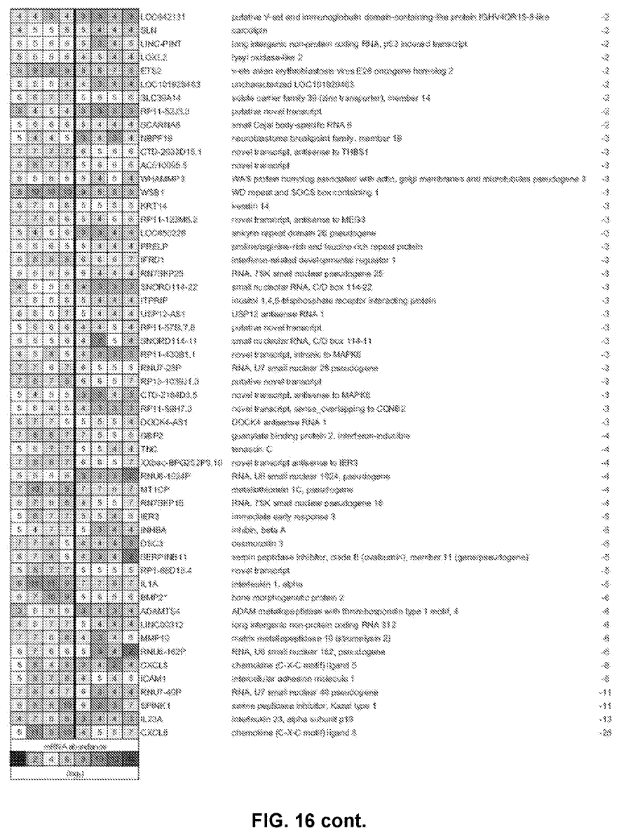

The clear boundary between the smooth chorion and the decidua parietalis enabled efficient laser microdissection of the latter cells. Comparison of heat maps of the mRNA samples that were isolated from sPE cases vs. nPTB controls revealed 227 genes that were DE in sPE by ≥2-fold (FIG. 3D and FIG. 16). The up regulated genes encoded molecules with immune functions such as PRG2 and KLRF1. Other genes in this category included RNASE2, PZP, PDGFD, NOTUM, and PROM1, which plays a role in the maintenance of adult stem cells. AOX1, which catalyzes the formation of superoxide and NO, was also up regulated, a possible sign of oxidative stress. At a pathway level, regulation of cell communication, several metabolic processes, and transmembrane receptor protein tyrosine kinase signaling among other pathways was significantly up regulated (FIG. 10).

The down regulated mRNAs included interleukins (CXCL8, IL23A, IL1A), CXCL5, as well as proteinases and their inhibitors (SPINK1, ADAMTS4 and MMP10), which play important roles during decidualization. At a pathway level, regulation of cell adhesion, locomotion and migration, morphogenesis, extracellular structure and immune processes were impacted (FIG. 10).

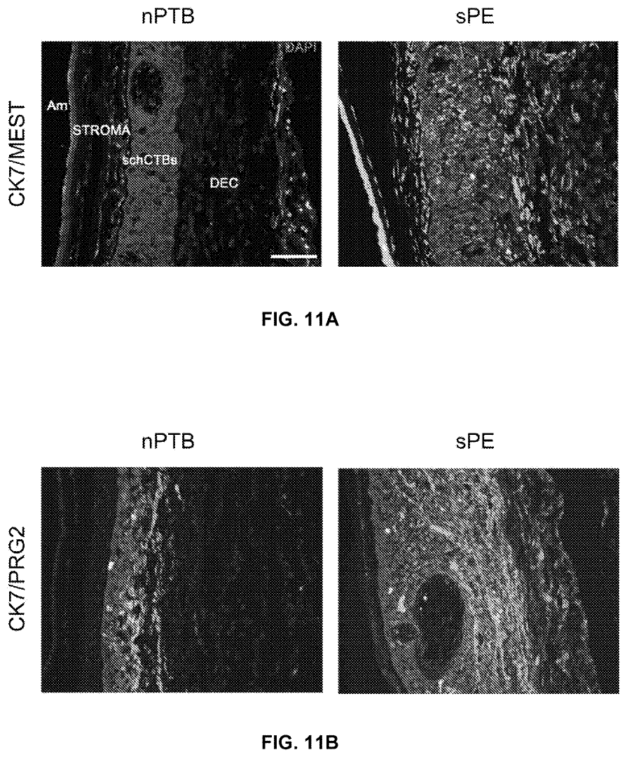

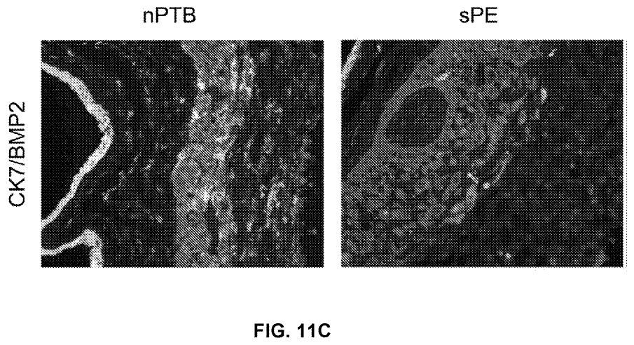

The microarray results were validated at the protein level for three DE genes (PEG1/MEST and PRG2, up-regulated in sPE; BMP2, down-regulated in sPE). In these experiments, an immunolocalization approach was applied to tissue sections of the fetal membranes with the adjacent decidua parietalis. In all cases, the protein-level results confirmed the expression patterns that were suggested by the transcriptomic data (FIGS. 11A-11C).

Thus, the global transcriptional profiling of the decidua basalis and the decidua parietalis as described herein revealed the differentially expressed genes (DEGs) in severe preeclampsia (sPE) vs. control pregnancies.

### Example 4

### Absence of Decidualization Markers in sPE Pregnancies

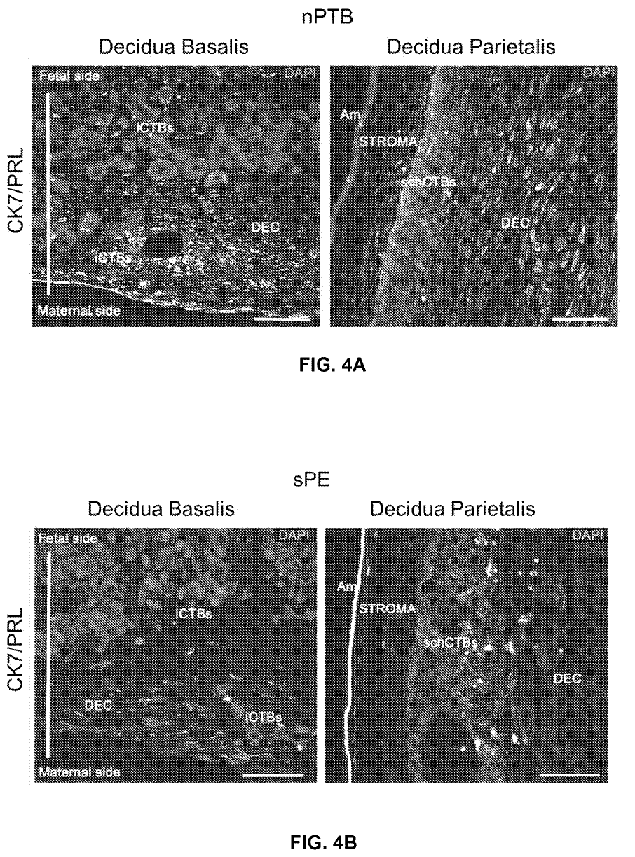

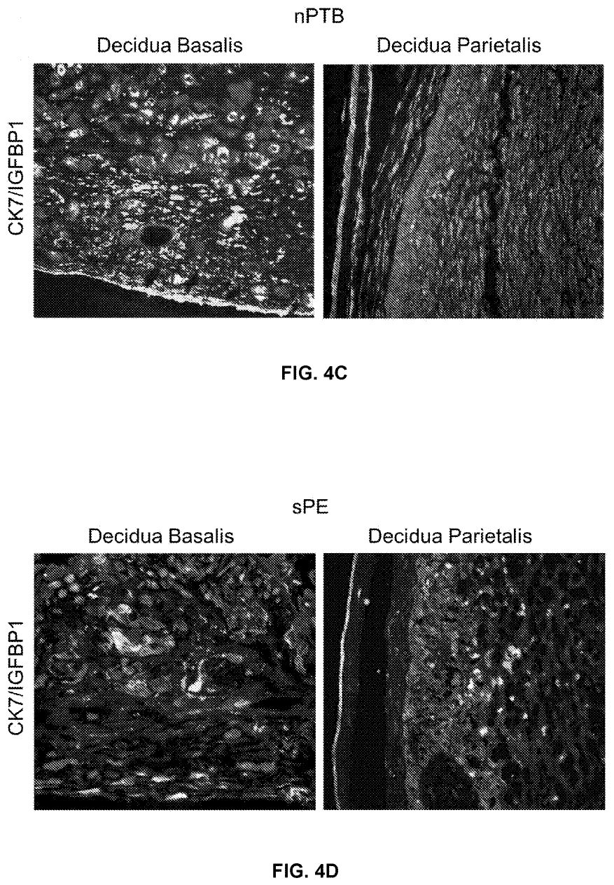

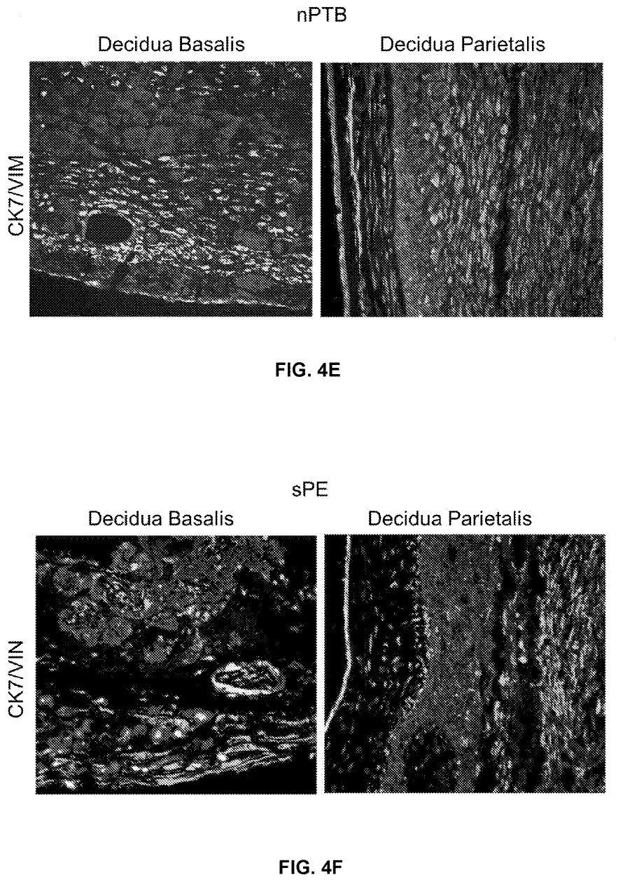

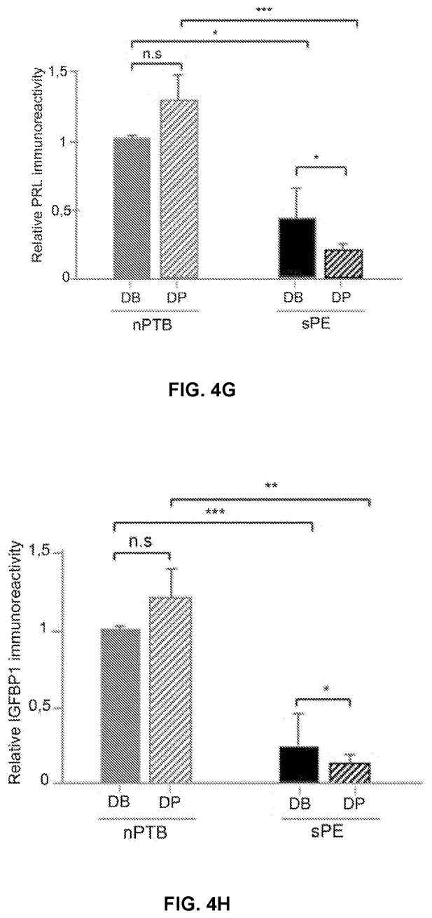

To analyze decidualization in situ, PRL (FIGS. 4A-4B) and IGFBP1 (FIGS. 4C-4D) expression in tissue sections of the decidua basalis and the decidua parietalis in sPE (n=5) was assessed as compared to nPTB (n=4). The clinical characteristics of the pregnancies are summarized in Table 9.

TABLE 9
Maternal and neonatal characteristics of
decidual donors (immunolocalization and
in vitro differentiation experiments).

| | nPTB | | sPE | | |
|---|---|---|---|---|---|
| | (n = 5) | | (n = 7) | | P** |
| Maternal Age (years) | 30.8 | (1.7) | 27.9 | (2.9) | >0.05 |
| Systolic blood pressure | 116.5 | (5.3) | 150.5 | (6.6) | <0.01 |
| (mmHg) | | | | | |
| Diastolic blood pressure | 68.2 | (4.6) | 88.0 | (3.6) | <0.01 |
| (mmHg) | | | | | |
| Proteinuria | 0 or NA | | + 1 to +2 | | <0.05 |
| Gestational age at delivery | 39.3 | (0.3) | 37.1 | (0.4) | >0.05 |
| (weeks) | | | | | |
| Birth weight (g) | 3110.1 | (177.3) | 2611 | (200.7) | <0.05 |

mean ± SEM
**One-tailed Student's t-test
NA: Not Available

Cytotrophoblast identity was confirmed by anti-cytokeratin 7 immunoreactivity (CK7; FIGS. 4A-4F) and stromal cells were visualized with anti-vimentin (VIM; FIGS. 4E-4F). The results showed that PRL and IGFBP1 were broadly expressed by decidualized stromal cells (basalis and parietalis) in control nPTB samples (FIGS. 4A and 4C). In contrast, expression of both decidualization markers was greatly reduced and in many instances absent in the sPE samples (FIGS. 4B and 4D). Relative immunoreactivity was quantified for PRL (FIG. 4G) and IGFBP1 (FIG. 4H).

Thus, the results demonstrate that sPE is associated with down-regulation of PRL and IGFBP1 expression in the decidua. The results also provided additional evidence that sPE is associated with widespread defects in decidualization that was evident in samples obtained immediately after delivery.

### Example 5

### Failure of Decidualization Marker Expression in Freshly Isolated Stromal Cells from sPE Decidual Biopsies

Decidual cells were isolated from sPE (n=5) or nPTB (n=4) cases with the goal of determining their status in terms of expressing stage-specific antigens that are typically associated with these cells.

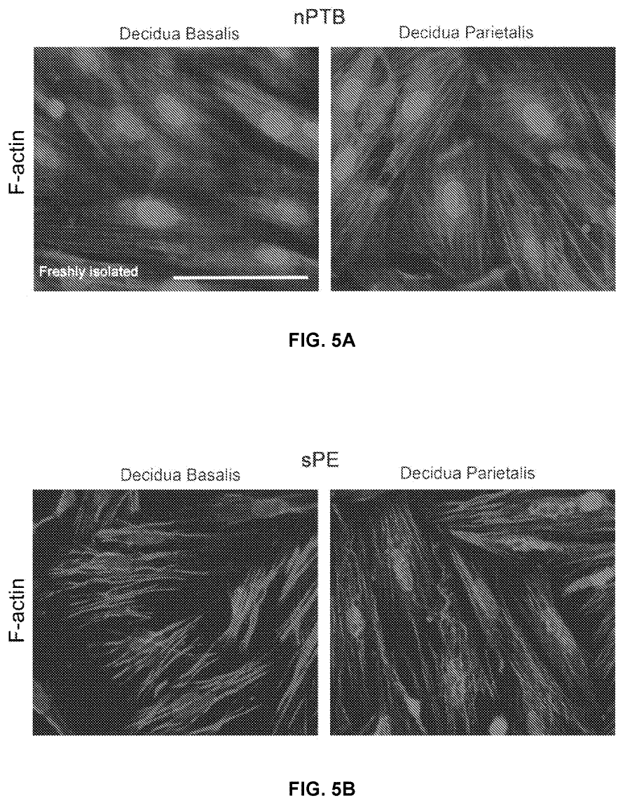

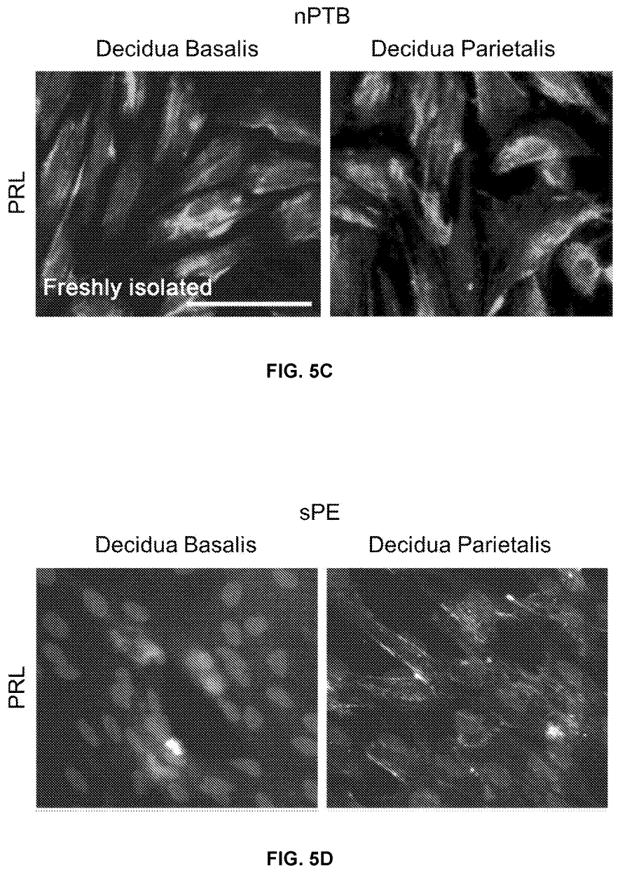

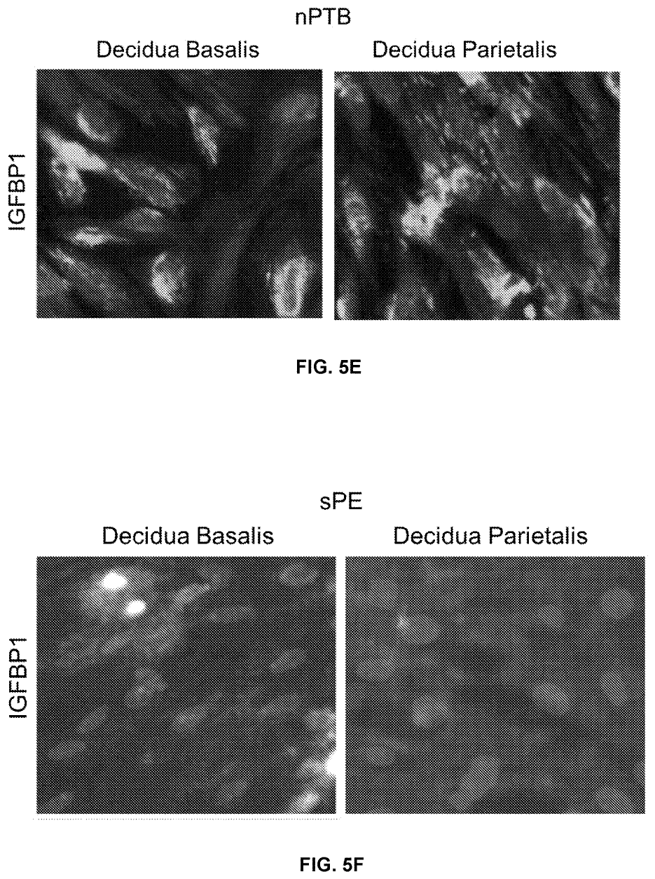

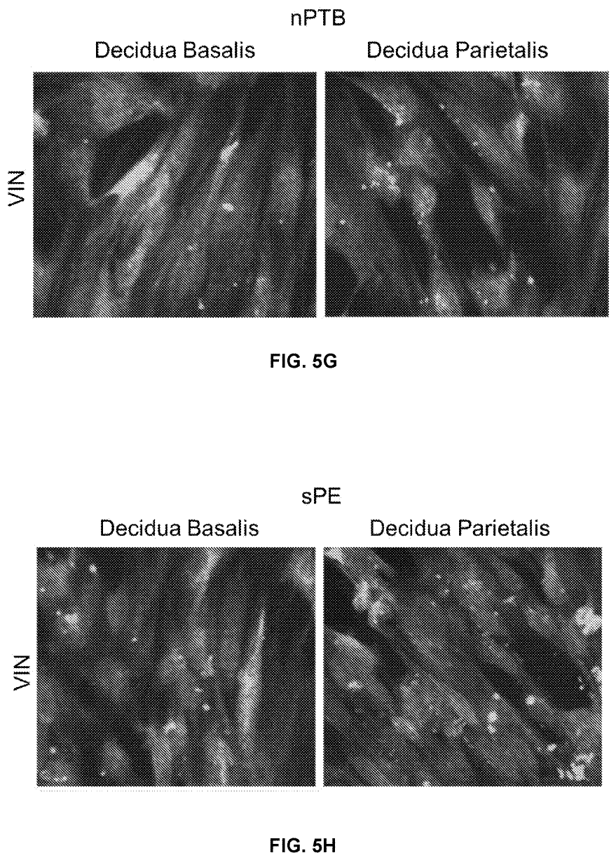

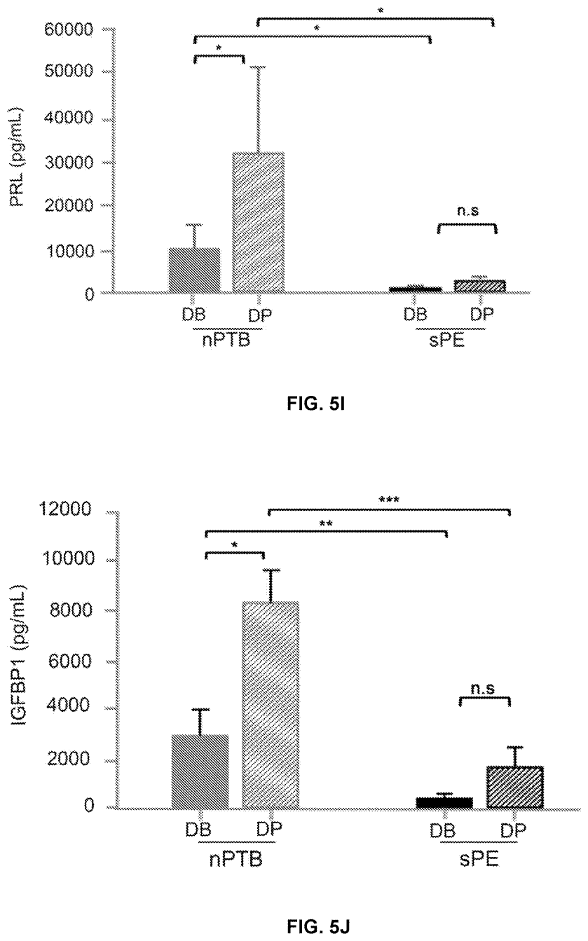

Freshly isolated stromal cells that were cultured overnight did not react with antibodies specific for markers of endothelial or hematopoetic cells, including macrophages (data not shown). Immediately after plating, rhodamine-phalloidin immunostaining showed the expected pattern of F-actin distribution in polygonal/round cells that were isolated from the decidua basalis or decidua parietalis from control nPTB samples (FIG. 5A). In contrast, stromal cells from decidual biopsies of sPE patients had an elongated morphology with a fibroblast-like F-actin organization (FIG. 5B). Immunostaining with anti-PRL (FIGS. 5C-5D) or anti-IGFBP1 (FIGS. 5E-5F) showed that stromal cells from sPE deciduas, whose identity was confirmed by vimentin expression (FIGS. 5G-5H), had much lower antibody reactivity than was observed in the control nPTB samples. Additionally, cellular secretion of PRL (FIG. 5I) and IGFBP1 (FIG. 5J) was quantified after overnight culture. In nPTB, production of both molecules was higher by cells isolated from the decidua parietalis as compared to the decidua basalis. In comparison, sPE was associated with a dramatic reduction in PRL and IGFBP1 secretion by cells isolated from both compartments.

Taken together, the results demonstrated that freshly isolated stromal cells from decidual biopsies of sPE patients displayed decidualization defects in culture.

### Example 6

### Failure of Stromal Cells Isolated from Decidual Biopsies obtained at Delivery from sPE Patients to Re-Decidualize In Vitro

To determine whether isolated stromal cells that were cultured for 3-5 passages could re-decidualize in vitro, morphological changes and secreted biomarkers (PRL and IGFBP1) were monitored after 5 days of hormone treatment.

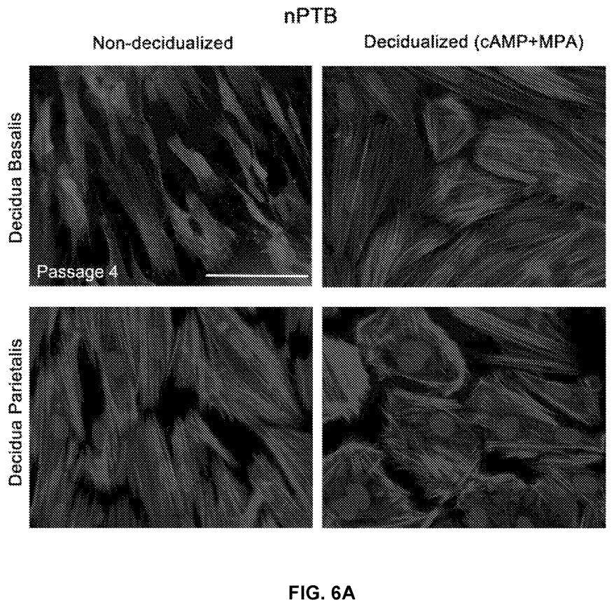

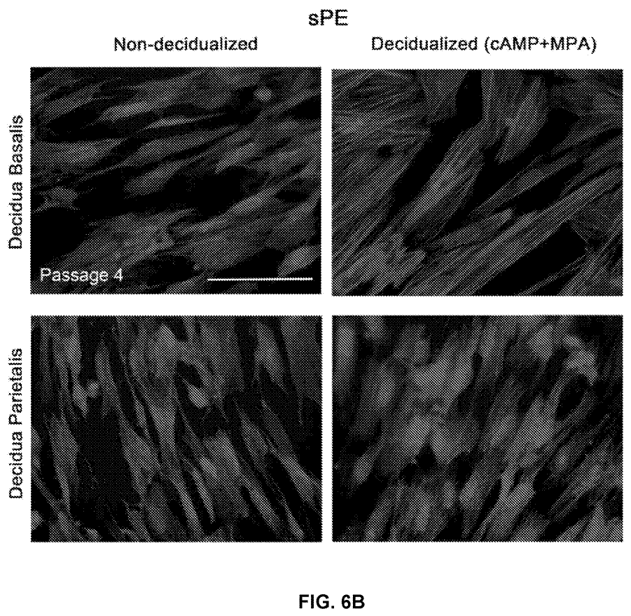

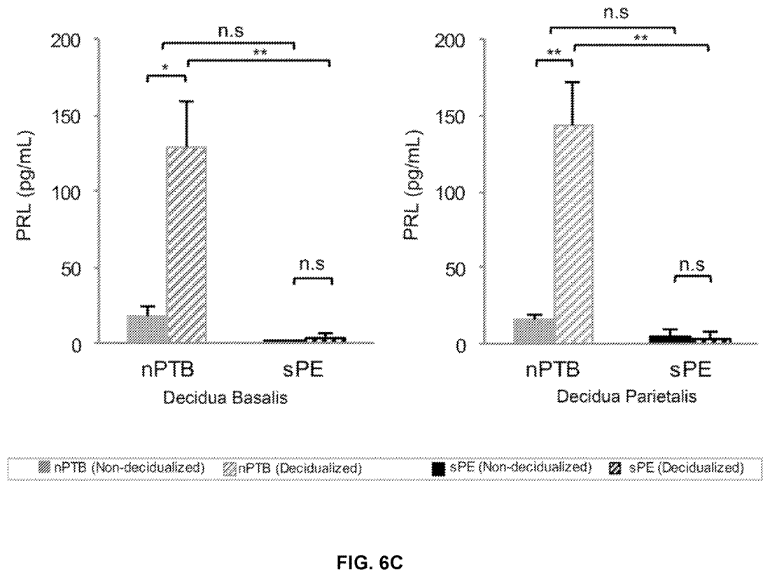

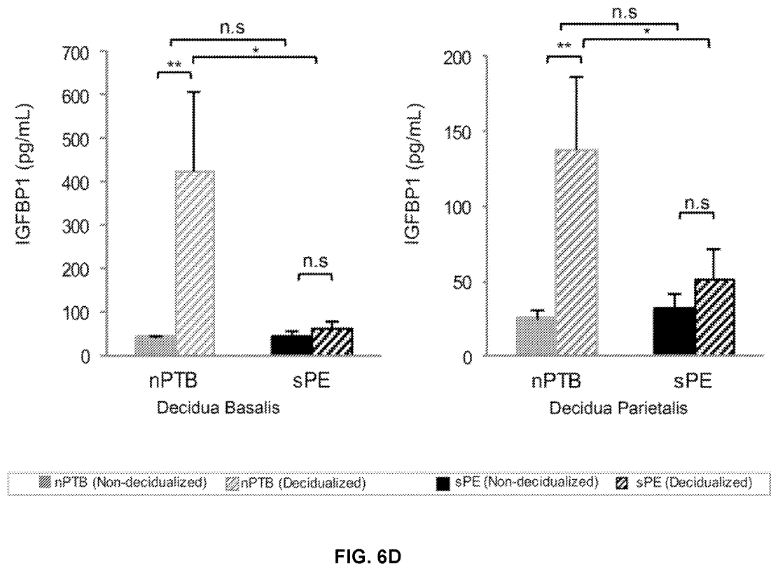

In cells from nPTB control patients, regardless of their compartment of origin, decidualization was associated with a characteristic polygonal/round phenotype as demonstrated by rhodamine-phalloidin immunostaining (FIG. 6A). In contrast, stromal cells from sPE patients failed to display morphological changes during re-decidualization (FIG. 6B). In control cells following decidualization, secretion of PRL (FIG. 6C) and IGFBP1 increased (FIG. 6D). In contrast, the equivalent cells isolated and cultured from sPE patients failed to increase secretion of either molecule (FIGS. 6C-6D) in response to MPA and cAMP treatment.

Taken together, the results demonstrated that cultured human endometrial stromal cells (hESCs) from decidual biopsies of sPE patients failed to re-decidualize in vitro.

### Example 7

### Failure of Conditioned Medium from sPE Decidual Cells to Promote Cytotrophoblast Invasion

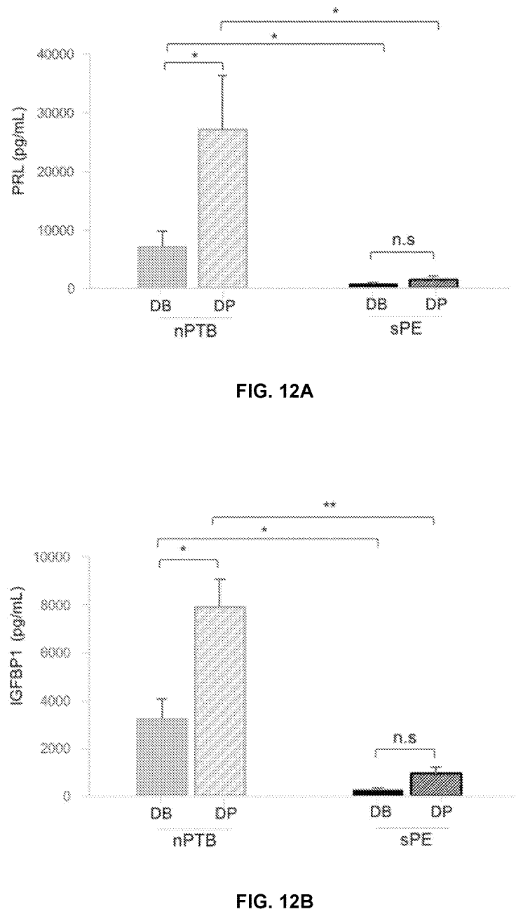

To determine whether the sPE-associated decidualization defect was related to reduced CTB invasion, stromal cells were isolated from samples of the decidua basalis and the decidua parietalis of sPE (n=4) or control nPTB (n=3) cases. Stromal cells were cultured overnight, and conditioned medium (CM) was collected. CM of sPE samples showed down regulation of PRL and IGFBP1 secretion compared to nPTB cultures (FIGS. 12A-12B). Accordingly, experimental or control CM was added to second trimester CTBs (n=10 placentas), which were cultured on a Matrigel substrate. Invasion was assayed by counting the number of CTBs or their cellular processes that reached the underside of the filters (FIG. 7A). In the presence of the control (nPTB) CM, robust invasion was observed, which was not statistically different between the CTBs that were cultured in the decidua basalis or the parietalis samples (FIG. 7B). In contrast, the CM from the equivalent stromal cell populations of sPE cases did not support CTB invasion.

To determine whether reduced PRL and IGFBP1 secretion by decidualized stromal cells from sPE patients was linked to the inability of CM from these cells to stimulate CTB invasion, cells were cultured in fresh medium. When the cells were cultured in fresh rather than conditioned medium, few CTBs reached the filter undersides (FIG. 7C) suggesting that nPTB decidual cells released factors promoting CTB invasion. The addition of PRL or IGFBP1 (10 ng/ml) to fresh medium had minimal effect. However, PRL and IGFBP1 in combination significantly increased invasion.

Thus, these results showed that conditioned medium from decidual cells of sPE patients inhibited cytotrophoblast (CTB) invasion in vitro.

### Discussion

As described herein, aberrant decidualization contributed to the phenotypic alterations in placentation that are associated with preeclampsia (PE). Human endometrial stromal cells (hESCs) were isolated from non-pregnant donors with a prior pregnancy that was complicated by severe PE (sPE). Compared to control cells, hESCs isolated from sPE patients failed to decidualize in vitro as demonstrated by morphological criteria and the analysis of stage-specific antigens (e.g., IGFBP1 and PRL). The results were confirmed by global transcriptional profiling data that showed hESCs isolated from sPE patients were transcriptionally inert. Additionally, laser microdissection was used to isolate the decidua from tissue sections of the maternal-fetal interface in sPE. Global transcriptional profiling revealed defects in gene expression. Further, decidual cells from sPE patients, which de-differentiated in vitro, failed to re-decidualize in culture. Conditioned medium from hESCs isolated from sPE patients failed to support CTB invasion, which was rescued by the combined addition of IGFBP1 and PRL. These data suggested that failed decidualization is an important contributor to down regulated CTB invasion in sPE.

### Example 8

### Global Transcriptional Profiling Corroborates an Endometrial Defective Decidualization Pattern in Severe Preeclampsia

Results discussed hereinabove demonstrate a defective in vitro decidualization of endometrial stromal cells isolated from patients with a previous severe preeclampsia (sPE) affecting the expression of 129 genes. To corroborate in vivo these findings, here, a global and targeted RNA sequencing was performed to identify this decidual endometrial defect during the secretory phase of the menstrual cycle in patients that have previously suffered sPE.

Prospective research trial where endometrial biopsies were obtained in the secretory phase from patients with a previous sPE pregnancy (n=16) versus controls with normal pregnancies outcomes that included preterm (n=10) and term (n=8) deliveries.

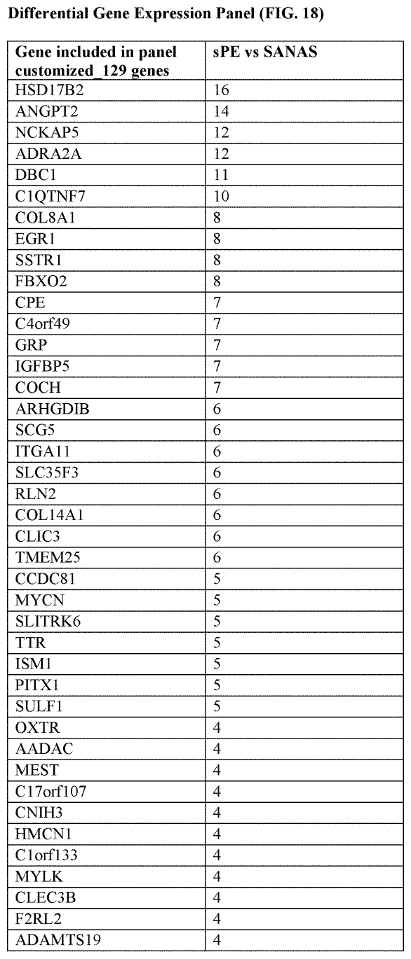

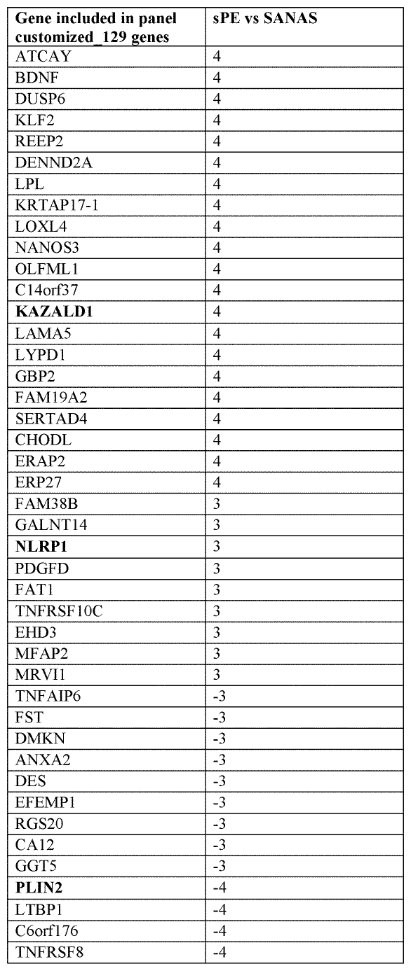

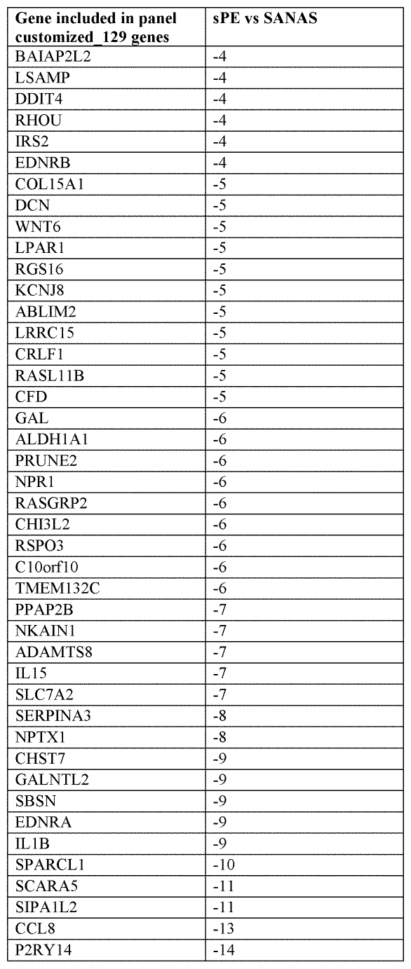

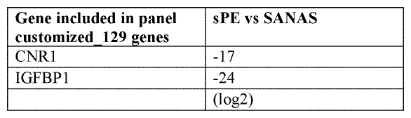

Both global gene profiling and targeted RNA sequencing were carried out using a custom panel designed for the 129 genes dysregulated (see Table 1 and FIG. 18). RNA was extracted using RNeasy mini-kit (Qiagen) and quality-checked by Fragment Analyzer (AATI, USA). Gene expression was analysed using a TruSeq Stranded mRNA in a NexSeq 500 platform (Illumina, USA) for global RNAseq and Ion AmpliSeq RNA in an Ion S5 system (Life Tech, USA) for the custom panel. All the sequences were pre-processed, normalized and analyzed comparing sPE vs control specimens (“SANAS”). Differentially expressed genes (DEGs) were determined by statistical analysis of FDR <0.05 and fold change ≥2.

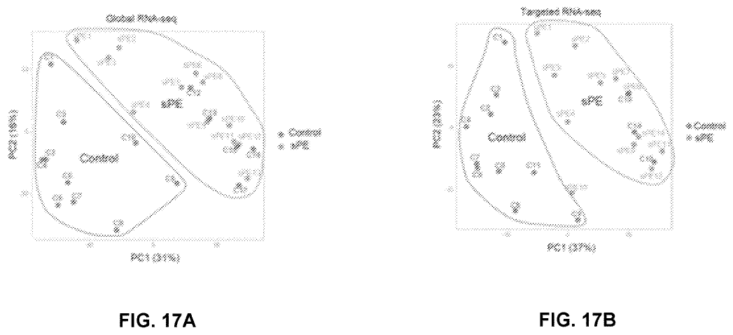

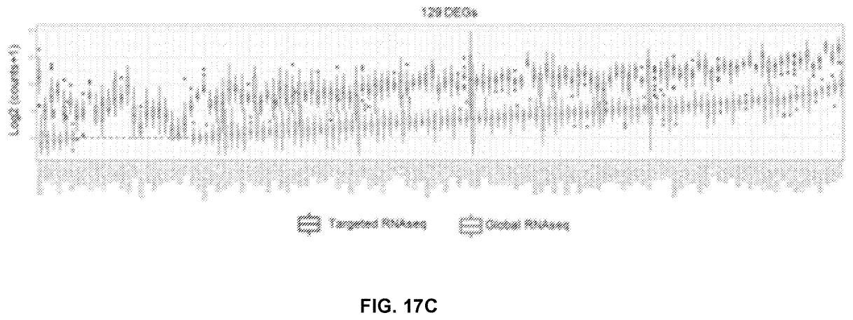

Global RNAseq analysis revealed 36 DEGs (see Table A) in the endometrium of patients with a prior sPE vs control pregnancies. Specifically, sample comparisons of sPE vs preterm control and sPE vs term control pregnancies showed 246 DEGs (see Table B) and 15 DEGs (see Table C), respectively. Interestingly, comparison between term and preterm control pregnancies failed to detect any difference in the gene profile. Principal component analysis (PCA) showed a separation between sPE vs control groups based on their transcriptional profiles. Strikingly, global and targeted RNAseq approaches obtained similar distribution of all the samples in the PCA (FIGS. 17A-17B). Correlation analysis revealed a strong gene expression association between the 129 DEGs targeted and those genes detected by global transcriptomic analysis (Pearson's value=0.89) (FIG. 17C).

Results using global gene profiling and targeted RNA sequencing corroborates the existence of an altered in vivo decidualization transcriptional profile in sPE patients. These findings further reinforce a possible maternal cause for sPE opening new directions to find strategies for early diagnosis and possible treatment.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

1. A method for detecting a level of at least one biomarker associated with preeclampsia in a sample from a subject, the method comprising
(a) determining a level of at least one biomarker in a sample obtained from a subject, wherein the at least one biomarker is selected from the group consisting essentially of:
(i) CNR1, IRS2, CHST7, PRUNE2, ADAMTS8, SCARA5, SERPINA3, NPR1, LPAR1, ABLIM2, CHI3L2, LTBP1, TNFRSF8, SLC27A3, ILI, CCDC, PPAP2C, SERTADA4, COCH, FBXO2, Clorf133, and CNIH3;
ii HSD17B2, ANGPT2, NCKAP5, ADRA2A, DBC1, C1QTNF7, COL8A1, EGR1, SSTR1, FBXO2, CPE, C4orf49, GRP, IGFBP5, COCH, ARHGDIB, SCG5, ITGA11, SLC35F3, RLN2, COL14A1, CLIC2, TMEM25, CCDC81, MYCN, NPR1, RASGRP2, CHI3L2, RSPO3, Cl0orf10, TMEM132C, PPAP2B, NKAIN1, ADAMTS8, IL15, SLC7A2, SERPINA3, NPTX1, CHST7, GALNTL2, SBSN, EDNRA, IL1B, SPARCL1, SCARA5, SIPA1L2, CCL8, P2RY14, CNR1, and IGFBP1;
(iii) A1BG-AS1, ARL5B, BAC1-AS, C7, COL8A1, CP, CSPG4, CYP19A1, DEFB1, ENPP4, IPW, LOC101928439, LOC101929607, LOC644172, MIR365A, MIR4509-1, MIR548H1, MME-AS1, MS4A2, OGN, PRKXP1, PSMD3, RNA5SP187, RNA5SP463, RNU2-5P, RNU4-39P, RNU4-76P, RNU4ATAC1BP, RNU6-1111P, RNU6-21P, RNU6-540R, RNU6V, RNUC-901P, RP11-1026M7.3, RP11-106K3.1, RP11-12D16.2, RP11-661Al2.4, RP11-872017.8, SNORD115-32, SNORD52, SNORD71, SPINK1, TAS2R46, TRAJ59, TRBV4-2, TRIM48, TSPAN1, UGT2B7, and ZNF483;
(iv) AC073218.2, AC073218.3, ACE2, ADAMTS15, ADAMTS4, AOX1, BMP2, CTC-498J12.1, CXCL5, CXCL8, DOCK4-AS1, DSC3, GBP2, GPR126, ICAM1, IER3, IGSF10, ILIA, IL23A, INHBA, KIR2DL2, KLRF1, LINC00312, LINCO1338, LOC100506530, LOC101929174, MMP10, MT1CP, MUM1L1, NOTUM, PDGFD, PRG2, PROM1, PZP, RN7SKP16, RNASE2, RNU6-162P, RNU7-40P, RNUC-1024P, RP11-57P19.1, RP11-59H7.3, RP1-68D18.4, SAPCD1, SERPIN811, SPINK1, SULF2, TMEM27, TNC, TRPC4, and Xxbac-BPG252F; or
(v) ADAMTS8, CHI3L2, CHST7, CNR1, COCH, FBXO2, NPR1, SCARA5, and SERPINA3; and
(b) determining that an absolute value of a ratio of the determined level of the biomarker in the sample to a control level of the biomarker is at least 2, thereby determining that the subject has or is at risk for preeclampsia.

2. The method of claim 1, further comprising treating the subject with an effective amount of an anti-preeclampsia therapy selected from the group consisting of an antihypertensive agent, an anticoagulant, a corticosteroid, an anticonvulsant, an antioxidant, a glycosaminoglycan, bed rest, hospitalization, maternal and fetal monitoring, and delivery.

3-4. (canceled)

5. The method of claim 1, wherein determining the level of a biomarker comprises performing an assay on a sample obtained from the subject.

6. The method of claim 1, wherein step (a) consists essentially of determining the level of at least five biomarkers from the group.

7-11. (canceled)

12. The method of claim 1, wherein step (a) consists essentially of determining the level of all biomarkers from the group.

13. (canceled)

14. The method of claim 1, wherein determining the level of a biomarker comprises determining the level of biomarker protein.

15. The method of claim 14, wherein the level of each biomarker protein is determined using an immunohistochemical assay, an immunoblotting assay, or a flow cytometry assay.

16. The method of claim 1, wherein determining the level of a biomarker comprises determining the level of biomarker nucleic acid.

17. The method of claim 16, wherein the level of each biomarker nucleic acid is measured by a real-time reverse transcriptase PCR (RT-PCR) assay or a nucleic acid microarray assay.

18. The method of claim 16, wherein the level of each biomarker nucleic acid is measured using a hybridization assay and at least one labeled binding agent.

19. The method of claim 18, wherein the at least one labeled binding agent is at least one labeled oligonucleotide binding agent.

20. The method of claim 1, wherein the sample is selected from the group consisting of a sample of endometrium tissue, endometrial stromal cells, and endometrial fluid.

21. The method of claim 1, wherein the sample is obtained from a human.

22. The method of claim 1, wherein the human is pregnant or is trying to become pregnant.

23-116. (canceled)

117. A method for detecting a level of at least one biomarker associated with preeclampsia in a sample from a subject, the method comprising
(a) determining a level of at least one biomarker in a sample obtained from a subject, wherein the at least one biomarker is selected from the group consisting essentially of: ADAMTS8, CHI3L2, CHST7, CNR1, COCH, FBXO2, NPR1, SCARA5, and SERPINA3; and
(b) determining that an absolute value of a ratio of the determined level of the biomarker in the sample to a control level of the biomarker is less than 2, thereby determining that the subject does not have preeclampsia.

118. The method of claim 117, wherein the method further comprises transferring one or more fertilized eggs or embryos to the subject.

119-139. (canceled)

140. A solid state assay device for determining the level of one or more biomarkers associated with preeclampsia, the device comprising:
a chip comprising one or more analysis regions,
wherein each analysis region consists essentially of a group of 5 to 129 binding partners,
and wherein each of the binding partners specifically binds to an expression product of a biomarker selected from FIGS. 14-16.

141-156. (canceled)

157. A kit comprising the solid state assay device of claim 140 and instructions for use.

158. A method for detecting a level of at least one biomarker associated with preeclampsia in a sample from a subject, the method comprising
(a) determining a level of at least one biomarker in a sample obtained from a subject, wherein the at least one biomarker is selected from at least one of the following pathways: extracellular structure organization, tissue development, inflammation, immune function, transport and/or metabolism, cell signaling, transcription and/or translation, signal transduction, protein degradation, insulin related, G-protein signaling, cell cycle and activation, and unspecified; and
(b) determining that an absolute value of a ratio of the determined level of the biomarker in the sample to a control level of the biomarker is at least 2, thereby determining that the subject has or is at risk for preeclampsia.

159-174. (canceled)

175. A method for detecting a level of at least one biomarker associated with preeclampsia in a sample from a subject, the method comprising
(a) determining a level of at least one biomarker in a sample obtained from a subject, wherein determining a level of at least one biomarker comprises a hybridization assay and at least one binding agent, and wherein the at least one binding agent is selected from the group consisting essentially of SEQ ID NOs.:1-8, and wherein the at least one biomarker is selected from the group consisting essentially of: ALDH1A1, IGFBP1, NANOS3, and HSD17B2; and
(b) determining that an absolute value of a ratio of the determined level of the biomarker in the sample to a control level of the biomarker is at least 2, thereby determining that the subject has or is at risk for preeclampsia.

176-177. (canceled)
